(19) United States
(12) Patent Application Publication
(10) Pub. No.: US 2012/0264685 A1
(43) Pub. Date: Oct. 18, 2012

(54) SHORT CHAIN PEPTIDOMIMETICS BASED ORALLY ACTIVE GLP 1 AGONIST AND GLUCAGON RECEPTOR ANTAGONIST

(76) Inventors: Rajesh Bahekar, Gujarat (IN); Mukul R. Jain, Gujarat (IN); Pankaj R. Patel, Gujarat (IN)

(21) Appl. No.: 13/502,826

(22) PCT Filed: Oct. 18, 2010
(86) PCT No.: PCT/IN2010/000683

§ 371 Date: Jul. 2, 2012

(30) Foreign Application Priority Data
Oct. 22, 2009 (IN) 2462/MUM/2009

(51) Int. Cl. C07K 7/08; A61P 17/02; A61P 3/10; A61P 3/00; A61K 38/10; A61P 3/06

(52) U.S. Cl. 514/7.2; 530/327; 514/21.5; 514/7.4; 514/6.8

(57) ABSTRACT

The present invention provides novel short chain peptidomimetics, which act as GLP-1 receptors agonist and glucagon receptor antagonist. These dual acting peptidomimetics exhibit increased stability to proteolytic cleavage, especially against DPP-IV (Dipeptidyl peptidase-IV) enzyme, GIT enzymes such as pepsin and acidic stomach pH and also against liver microsomes (in vitro). Due to increased metabolic stability, other than parenteral route of administration, these short chain peptidomimetics can be delivered by oral routes of administration, for the treatment or prevention of diabetes and related metabolic disorders, such as obesity, hyperlipidemia and eating disorders.

### FIELD OF THE INVENTION

The present invention relates to novel compounds of general Formula (I), their tautomeric forms, pharmaceutically acceptable salts, pharmaceutical acceptable solvates, and pharmaceutical compositions containing them. The invention also relates to processes for preparing the compounds of general Formula (I), their tautomeric forms, pharmaceutically acceptable salts, pharmaceutical acceptable solvates, and pharmaceutical compositions containing the compounds and to methods for treating diabetes and metabolic disorders using the compounds of the invention.

A-β-C-D (I)

The present invention describes a group of novel peptidomimetics that function both as an antagonist of the glucagon receptor and agonist of the GLP-1 receptor, having different degree of affinity/selectivity towards both the receptors and useful for reducing circulating glucose levels and for the treatment of diabetes and metabolic disorders.

The compounds of general formula (I) are useful in the treatment of the human or animal body, by regulation of insulin and glucagon action. Thus, it can be used in the treatment of a variety of conditions and disorders, including, but not limited to, treating or delaying the progression or onset of diabetes (preferably type II, impaired glucose tolerance, insulin resistance and diabetic complications, such as nephropathy, retinopathy, neuropathy and cataracts), hyperglycemia, hyperinsulinemia, hypercholesterolemia, elevated blood levels of free fatty acids or glycerol, hyperlipidemia, hypertriglyceridemia, wound healing, tissue ischemia, atherosclerosis, hypertension, intestinal diseases (such as necrotizing enteritis, microvillus inclusion disease or celic disease).

The compounds of general Formula (I) are useful for the treatment of Syndrome X or metabolic disorders.

### BACKGROUND OF THE INVENTION

Metabolic disorders such as diabetes, obesity and its associated consequences remain a primary health and economic threat for modern societies. Diabetes is characterized by impaired insulin secretion from pancreatic β-cells, insulin resistance or both (Cavaghan, M. K., et al., J. Clin. Invest. 2000, 106, 329). Majority of type 2 diabetic patients can be treated with agents that reduce hepatic glucose production (glucagon antagonist), reduce glucose absorption form GIT, stimulate β-cell function (insulin secretagogues) or with agents that enhance the tissue sensitivity of the patients towards insulin (insulin sensitizes). The drugs presently used to treat type 2 diabetes include α-glucosidase inhibitors, insulin sensitizers, insulin secretagogues and K_{ATP} channel blocker (Chehade, J. M., et al., Drugs, 2000, 60, 95). However, almost one-half of the type 2 diabetic subjects lose their response to these agents, over a period of time and thereby require insulin therapy. Insulin treatment has several drawbacks; it is injectable, produces hypoglycemia and causes weight gain (Burge, M. R., Diabetes Obes. Metab., 1999, 1, 199).

Problems with the current treatment necessitate new therapies to treat type 2 diabetes. In this regard, glucagon-like peptide-1 (GLP-1) agonist, which promote glucose-dependent insulin secretion in the pancreas and glucagon receptor antagonist, which inhibit hepatic glucose production by inhibiting glycogenolysis and gluconeogenesis, were found to be therapeutically potential. Thus GLP-1 agonist and glucagon antagonist together were found to reduce the circulating glucose levels and represent useful therapeutic agents for the treatment and prevention of type 2 diabetes (Perry, T. A., et al., Trends Pharmacol. Sci., 2003, 24, 377).

Glucagon and GLP-1 are members of structurally related peptide hormone family (secretin family). Glucagon and GLP-1 constitute a highly homologous set of peptides because these two hormones originate from a common precursor, preproglucagon, which upon tissue-specific processing leads to production of GLP-1 predominantly in the intestine and glucagon in the pancreas (Jiang, G., et al., Am. J. Physiol. Endocrinol. Metab., 2003, 284, E671-678). The receptors for these two peptides are homologous (58% identity) and belong to the class B family of G-protein coupled receptors (GPCRs). Class-B GPCRS is also called as the secretin receptor family, which consist of 15 peptide-binding receptors in humans. GPCR receptors comprise an extracellular N-terminal domain of 100-160 residues, connected to a juxtamembrane domain (J-domain) of seven membrane-spanning co-helices with intervening loops and a C-terminal tail (Brubaker, P. L., et al., Receptors Channels, 2002, 8, 179). Class B GPCRs are activated by endogenous peptide ligands of intermediate size, typically 30-40 amino acids (Hoare, S. R. J., Drug Discovery Today, 2005, 10, 423; Gether, U., Endocrine Reviews, 2000, 21, 90).

Glucagon is a 29-amino acid peptide hormone processed from proglucagon in pancreatic α-cells by PC2. Glucagon acts via a seven transmembrane GPCRs, consisting of 485 amino acids. Glucagon is released into the bloodstream when circulating glucose is low. The main physiological role of glucagon is to stimulate hepatic glucose output, thereby leading to increase in glycemia (Tan, K., et al., Diabetologia, 1985, 28, 435). Glucagon provides the major counter regulatory mechanism for insulin in maintaining glucose homeostasis in vivo. Glucagon and its receptor represent potential targets for the treatment of diabetes. Antagonising glucagon action by blocking the action of the secreted glucagon at glucagon receptor (glucagon antagonist) or by inhibiting (suppressing) the glucagon production itself represents a new avenue for intervention of diabetes and metabolic disorders (Unson, C. G., et al., Peptides, 1989, 10, 1171; Parker, J. C., Diabetes, 2000, 49, 2079; Johnson, D. G., Science, 1982, 215, 1115).

The GLP-1 (7-36) amide is a product of the preproglucagon gene, which is secreted from intestinal L-cells, in response to the ingestion of food. The physiological action of GLP-1 has gained considerable interest. GLP-1 exerts multiple actions by stimulating insulin secretion from pancreatic β-cells, in a glucose dependent manner (insulinotropic action). GLP-1 lowers circulating plasma glucagon concentration, by inhibiting its secretion (production) from α-cells (Drucker D. J., Endocrinology, 2001, 142, 521-527). GLP-1 also exhibits properties like stimulation of β-cell growth, appetite suppression, delayed gastric emptying and stimulation of insulin sensitivity (Nauck, M. A., Horm. Metab. Res., 2004, 36, 852). Currently, various analogs of GLP-1 and EX-4, such as Liraglutide/NN2211 (Novo Nordisk; Phase-III; WO 1998 008871), BIM 51077 (Ipsen; Phase-II; WO 2000 034331), CJC-1131 (ConjuChem; Phase-II; WO 2000 069911) and ZP-10 (Zealand & Aventis; Phase-II; WO 2001 004156) are in different stages of clinical development (Nauck M. A., Regulatory Peptides, 2004, 115, 13). Recently, BYETTA® (Exendin-4, AC 2933; U.S. Pat. No. 5,424,286), has been launched in the US market (Amylin & Lilly). However, all the existing GLP-1 agonists are delivered by the parenteral route of administration, so the patient in compliance is a major problem with the existing GLP-1 based therapy.

The effector system of glucagon and GLP-1 receptors is the Adenylyl Cyclase (AC) enzyme. Interaction of glucagon antagonist or GLP-1 agonist with glucagon or GLP-1 receptors (GLP-1 R) respectively causes activation of AC, which converts ATP to cAMP. Increase in the intracellular cAMP level raises the ratio of ADP/ATP, thereby initiating the cell depolarization (due to closure of K_{ATP} channel). Increase in the intracellular cAMP level also activates Protein Kinase (PK-A & PK-C), which raises the cystolic Ca²⁺ concentration, by opening of L-type of Ca²⁺ channel. An increase in the intracellular Ca²⁺ leads to exocytosis of insulin, in pancreatic β-cells and glucagon peptide in α-cells (Fehmann, H. C., Endocr. Rev., 1995, 16, 390).

GLP-1 and glucagon sequences alignment shown below represent the primary structural relationships:

Glucagon:
(Seq. ID No: 1)
NH₂-¹HSQGTFTSD⁹YSKYLDSRRAQDFVQWLMNT-CONH₂
GLP-1 (7-36):
(Seq. ID No: 2)
NH₂-¹HAEGTFTSD⁹VSSYLEGQAAKEFIAWLVKGR-CONH₂
First N-terminal 1-9 residues of GLP-1 peptide:
(Seq. ID No: 3)
¹HAEGTFTSD⁹
First N-terminal 1-9 residues of Glucagon peptide,
(Seq. ID No: 4)
¹HSQGTFTSD⁹

Single-letter abbreviations for amino acids can be found in Zubay, G., Biochemistry 2^{nd} ed., 1988, MacMillan Publishing, New York, p. 33.

Native or synthetic GLP-1 peptides are rapidly metabolized by the proteolytic enzymes, such as dipeptidyl peptidase-IV (DPP-IV) into an inactive metabolite, thereby limiting the use of GLP-1 as a drug (Deacon, C. F., Regulatory Peptides, 2005, 128, 117). Similarly, several nonpeptidyl and peptidyl glucagon receptor antagonist of diverse structures have been reported over recent years, but none of them are in active development or under clinical trials (Kurukulasuriya, R., Expert Opinion Therapeutic Patents, 2005, 15, 1739; Lau, J., J. Med. Chem., 2007, 50, 113; Petersen, K. F. Diabetologia, 2001, 44, 2018; Cascieri, M. A., JBC, 1999, 274, 8694). It is believed that identifying nonpeptide ligands (especially agonist) for class B GPCRs is the principle bottleneck in drug discovery. HTS has apparently yielded few hits (US 2005/6927214; WO 2000/042026; US 2007/0043093), however, screening of those hits against corresponding receptors, especially under in vivo condition (animal models) prone to be false negatives (Murphy, K. G., PNAS, 2007, 104, 689).

Glucagon and GLP-1 both play major roles in overall glucose homeostasis (Drucker, D. J., J. Clin. Invest., 2007, 117, 24; Bollyky, J., J. Clin. Endocrinol. Metab., 2007, 92, 2879). Glucagon increases plasma glucose concentrations by stimulating gluconeogenesis and glycogenolysis in the liver while GLP-1 lowers plasma glucose concentrations mediated by glucose dependent insulin secretion (Mojsov, S., et al., JBC., 1990, 265, 8001). Knowing the importance of both glucagon peptide and GLP-1 in maintaining normal blood glucose concentrations, in the recent years, there has been considerable interest in identifying a single ligand, which act as glucagon receptor antagonists and GLP-1 receptor agonists (Claus, T. H., J. Endocrinology, 2007, 192, 371; Pan C. Q., JBC, 2006, 281, 12506).

Although identification of potent nonpeptide GLP-1 agonist may be difficult (Chen, D., PNAS, 2007, 104, 943; Knudsen, L. B., PNAS, 2007, 104, 937) but the design of a hybrid peptidomimetic acting as both glucagon antagonist and GLP-1 receptor agonist would likely to provide a novel approach for the treatment of type 2 diabetes (Claus, T. H., J. Endocrinology, 2007, 192, 371). Structure-activity relationship (SAR) studies have been reported in the literature to determine the role of individual amino acids in both the glucagon and GLP-1 sequences (Runge, S., JBC, 2003, 278, 28005; Mann, R., Biochem. Soc. Trans., 2007, 35, 713). Glucagon and GLP-1 have no defined structure in aqueous solution, but in the presence of micelles or in the membrane mimetic environment, they adopt an alpha-helical structure in the mid-section, with flexible N- and C-terminal regions (Thornton, K., Biochemistry, 1994, 33, 3532; Neidigh, J. W., Biochemistry, 2001, 40, 13188). This suggests that the helical structure is required for binding of peptide ligands to their respective receptors. Mutations or deletion of amino acids in the N-terminal region of both the peptides results in receptor antagonists or inactive compounds, suggesting the importance of the N-terminus for receptor activation by both the glucagon and GLP-1 peptides (Hjorth, S. A., JBC., 1994, 269, 30121; Green, B. D., J. Mol. Endocrinology, 2003, 31, 529). In vivo, GLP-1 gets rapidly degraded by dipeptidyl-peptidase IV (DPP IV), a protease responsible for cleaving peptides containing proline or alanine residues in the penultimate N-terminal position, resulting in the inactive metabolites. Substitution of the DPP-IV susceptible sites, such as substitution of Ala at 2^{nd} position of GLP-1 peptide with D-Ala, Aib, greatly improves plasma stability (Deacon, C. F., Diabetes, 1998, 47, 764).

Oral delivery of peptide based drug is mainly limited due to its rapid hydrolysis and reduced permeability from the intestinal lumen (X. H. Zhou, J. Control Release, 29, 1994, 239). Optimization of such chemical entities for human oral bioavailability is generally confounded by molecules that are substrates for intestinal uptake or efflux transporters. The human intestinal small peptide carrier (hPEPT1) is a proton-coupled, oligopeptide transport system with broad substrate specificity (G. Kottra, et al., J. Biol. Chem., 277(36), 2002, 32683; R. Liang, et al., J. Biol. Chem., 270, 1995, 6456). In the small intestine, the population of PEPT1 increases from the duodenum to the ileum (H. Tanaka, et al., Gastroenterology, 114, 1998, 714). In addition to transporting its natural substrates, di- and tri-peptides occurring in food products, PEPT1 shows affinity towards a broad range of peptide-like pharmaceutically relevant compounds, such as β-lactam antibiotics and angiotensin converting enzyme (ACE)-inhibitors (F. H. Leibach and V. Ganaphthy, Annu. Rev. Nutr., 16, 1996, 99-119; H. Daniel and M. Herget, Am. J. Physiol., 273, 1997, F1; M. E. Ganaphthy, et al., J. Biol. Chem., 270, 1995, 25672).

The PEPT1 has been used as a target molecule for improving the intestinal absorption of poorly absorbed drugs through amino acid modifications, for example, the enhanced oral bioavailability of Midodrine (Gly prodrug of 1-(2′,5′-dimethoxyphenyl)-2-aminoethanol (DMAE)) has been attributed to their enhanced intestinal transport via PEPT1 (M. Tsuda., et al., J. Pharmacol. Exp. Ther., 318, 2006, 455). Temple et al., reported 4-aminophenylacetic acid as a peptide mimic substrate for the PEPT1 (C. S. Temple, et al., J. Biol. Chem., 273(1), 1998, 20). Valacyclovir is the 5′-valyl ester prodrug of acyclovir. Systemic availability of acyclovir in humans is 3-5 times higher when administered orally as the prodrug. The increased bioavailability of valacyclovir is attributed to carrier-mediated intestinal absorption, via the hPEPT1 peptide transporter (P. V. Balimane, et al., Biochem. Biophys. Res. Commun., 250, 1998, 246). Similarly, dipeptide ester prodrug (Gly-Val-ACV) of acyclovir (ACV) possess high affinity toward the intestinal hPEPT1 (B. S. Anand, et al., JPET., 311, 2004, 659). For this reason, PEPT1 transporter system has been recognized as an important component in the oral bioavailability of small peptides and peptidomimetic compounds (B. S. Vig, et al., J. Med. Chem., 49, 2006, 3636).

The present invention provides novel short chain peptidomimetics of Formula (I) (hereinafter referred to as peptidomimetics), which primarily act as a glucagon receptor antagonist and also exhibit GLP-1R agonistic effects. Different peptidomimetics reported in this invention showed significant glucose dependent insulin secretion (in vitro) and reduce circulating glucose levels (in vivo), with different level of affinity/selectivity towards glucagon and GLP-1 receptors. Furthermore, these peptidomimetics showed increased stability to proteolytic cleavage, especially against DPP-IV enzyme with improved half-life. These novel peptidomimetics were found to be stable against GIT enzymes and acidic pH of stomach, with improved oral bioavailability making them suitable candidate for the treatment/mitigation/prophylaxis of both type 1 and type 2 diabetes, metabolic disorders and related disorders. A series of human GLP-1 mimics, have been reported with general formula Xaa1-Xaa11, wherein Xaa1-Xaa9 represent the first 1-9 residues of GLP-1 peptide (HAEGTFTSD; Seq. ID No. 3), with some analogs wherein Xaa2 represents either Ala or are optionally replaced with Aib, Xaa3 represents amino acids with carboxylic acid side chain such as glutamic acid, aspartic acid etc. but not the Gln (O), which is conserved in N-terminal sequence of Glucagon peptide (HSQGTFTSD, Seq. ID No. 4). Xaa6 represents Phe or are optionally replaced with -α-Me-2F-Phe- or -α-Me-2,6-F-Phe-, Xaa9 represent amino acids with carboxylic acid or amide side chains such as aspartic acid, glutamic acid, asparagine etc., Xaa10 & Xaa11 represents combination of substituted or unsubstituted biphenylalanine (Bip) or 2-Amino-5-phenyl-pentanoic acid (APPA) derivatives (WO 2003/033671A2; US 2004/0127423 A1; WO 2004/094461 A2; US 2006/0004222 A1; WO 2006/014287 A1; WO 2006/127948 A2; WO 2007/082264 A2; US 2007/0021346 A1; US2007/0099835; US 2007/0238669A1).

Earlier, we reported novel peptidomimetics of formula A-Z₁-to-Z₁₁-α, as glucose dependent insulin secretagogues, glucagon receptor antagonist and GLP-1 receptor agonist (WO 2008/062457; WO 2009/125424), wherein, the N-terminal sequence of glucagon peptide (first 1-9 residues, Seq. ID. No. 4) was coupled with the dipeptide of two unnatural amino acids resulted in the identification of novel class of peptidomimetics having both the glucagon antagonistic and GLP-1 agonistic activities, at varying degree of selectivity.

Oral delivery of peptide-based drug is mainly limited due to its poor metabolic stability and reduced permeability from the intestinal lumen (X. H. Zhou, J. Control Release, 29, 1994, 239). Knowing the fact that the PEPT1 peptide transporter showed affinity towards amino acid based substrates, mainly amino acids such as Gly, Val, Ala or their di and tri peptides (D. Meredith, et al., Eur. J. Biochem., 267, 2000, 3723; P. D. Bailey, et al., Angew Chem. Int. Ed. Engl., 39, 2000, 505), we herein disclose, novel short-chain peptides/peptidomimetics of formula (I) (A-B—C-D), in which ‘A’ represent PEPT1 transporter substrate, consisting of amino acids such as Val (V) or Lys (K) or Leu (L) or Ala (A) or Aib or their di-peptides; ‘B’ represent the first N-terminal 1-9 residues (formula II: Z₁—Z₂—Z₃—Z₄—Z₅—Z₆—Z₇—Z₈—Z₉) of GLP-1 (HAEGTFTSD; Seq. ID No. 3) or Glucagon peptide (HSQGTFTSD, Seq. ID No. 4), wherein, Z₁-to-Z₉ are optionally replaced with homologues unnatural amino acids to improve its metabolic stability; ‘C’ represent dipeptide of formula (III; Z₁₀—Z₁₁), which mainly act as binding component and ‘D’ represent C-terminus of short-chain peptide/peptidomimetics.

Due to increased metabolic stability (configure by incorporation of unnatural amino acids) and epithelial permeability (induce by introduction of PEPT1 transporter substrate), several of these short-chain peptides are active via oral route of administration.

The present invention also describes a group of novel peptidomimetics of Formula (I) that function both as an antagonist of the glucagon receptor and agonist of the GLP-1 receptor, having different degree of affinity/selectivity towards both the receptors and useful for reducing circulating glucose levels and for the treatment of diabetes and metabolic disorders and method for their preparation.

### SUMMARY OF THE INVENTION

In one aspect there are provided novel short-chain peptides/peptidomimetics represented by general Formula (I),

A-B—C-D (I)

and their tautomeric forms, pharmaceutically acceptable salts, pharmaceutical acceptable solvates, and pharmaceutical compositions containing them.

In another aspect of the invention there are provided processes for the preparation of compounds represented by the general Formula (I), and their tautomeric forms, pharmaceutically acceptable salts, pharmaceutical acceptable solvates, and pharmaceutical compositions containing them or their mixtures.

In another aspect of the invention there are provided pharmaceutical compositions containing compounds of the general Formula (I), and their tautomeric forms, novel intermediates involved in the synthesis, pharmaceutically acceptable solvates, pharmaceutically acceptable salts in combination with suitable carriers, excipients, or diluents or other media normally employed in preparing such compositions, which can be used for the treatment/mitigation/regulation or prophylaxis of type 1 & type 2 diabetes and associated metabolic disorders.

In another aspect of the invention there is provided the use of the novel peptidomimetics as antidiabetic agents, by administering a therapeutically effective & non-toxic amount of the peptidomimetics of Formula (I), or their pharmaceutically acceptable compositions to the mammals those are in need of such treatment

The details of one or more embodiments of the inventions are set forth in the description below. Other features, objects and advantages of the inventions will be apparent from the description.

### BRIEF DESCRIPTION OF DRAWINGS

FIG. 1 illustrates examples of orthogonally protected amino acids used in Fmoc based-solid phase peptide synthesis (SPPS) of peptidomimetics.

FIG. 2 illustrates in vitro human Glucagon receptor antagonistic activity and GLP-1 receptor agonistic activity with Seq. ID. 121.

FIG. 3 illustrates in vivo glucose reduction in C57 mice, with Seq. ID No. 102, after intraperitonial (i.p) administration.

FIG. 4 illustrates in vivo glucose reduction in C57 mice, with Seq. ID No. 102, after oral (p.o) administration.

FIG. 5 illustrates in vivo glucose reduction in db/db mice, with Seq. ID No. 102, after oral (p.o) administration.

FIG. 6 illustrates the serum insulin levels after single oral administration of vehicles/test peptidomimetics (Seq. ID. No. 29, 54 and 82), in ob/ob mice (in vivo).

### ABBREVIATIONS USED

The following abbreviations are employed in the examples and elsewhere herein:
AC₃C=1-amino cyclopropane carboxylic acid,
AC₅C=1-amino-cyclopentanecarboxylic acid,
αMe-Asp=alpha-methyl-Aspartic acid,
αMe-APPA=α-methylated APPA,
αMe-Bip(OMe)=α-methylated Bip(OMe),
αMe-Gln=alpha-methyl-Glutamine,
αMe-Glu=alpha-methyl-Glutamic acid,
αMe-His=α-methyl-Histidine,
αMe-Phe=alpha-methyl-phenylalanine,
αMe-2F-Phe=alpha-methyl-2-fluorophenylalanine,
αMe-2,6-F-Phe=alpha-methyl-2,6-difluorophenylalanine,
αMe-Pro=α-methyl proline,
αMe-Ser=alpha-methyl-Serine,
Ser(OMe)=Serine(OMe)-OH,
αMe-Ser(OMe)=alpha-methyl-Serine(OMe)-OH,
αMe-Thr=alpha-methyl-Threonine,
Aib=α-Aminoisobutyric acid,
ACN=Acetonitrile,
APPA=2-Amino-5-phenyl-pentanoic acid,
Bn=Benzyl,
Boc=tert-Butoxycarbonyl,
Bu^{t}=O-tert-butyl group,
Bip(OMe)=2′-ethyl-4′-methoxy-biphenylalanine,
cAMP=Adenosine 3′,5′-cyclic monophosphate,
DCM=Dichloromethane,
DMF=N,N-Dimethylformamide,
DIPCDI=Di-isopropylcarbodiimide,
DIPEA=Diisopropylethylamine,
Et=Ethyl,
Et₂O=Diethyl ether,
2F-Phe=2-fluorophenylalanine,
Fmoc=Fluorenylmethoxycarbonyl,
g=Gram (s),
GLP-1R=Glucagon Like Peptide-1 Receptor,
Glucagon R=Glucagon receptor,
h=Hour (s),
HOBt=Hydroxybenzotriazole,
HOAt=7-Aza-hydroxybenzotriazole,
HBTU=2-(1H-benzotriazole-1-yl)-1,1,3,3-tetramethyl aminium hexafluorophosphate,
HPLC=High Performance Liquid Chromatography,
i.p.=intraperitonial,
L=Liter,
LC/MS=Liquid Chromatography/Mass Spectrometry,
Me=Methyl,
Min=minute (s),
mL=milliliter,
μl=microliter,
mg=milligram (s),
mmol=millimole (s),
MS=Mass Spectrometry,
NMe-Asp=N-methyl-Aspartic acid,
NMe-Gln=N-methyl-Glutamine,
NMe-Glu=N-methyl-Glutamic acid,
NMe-Ser=N-methyl-Serine,
NMe-Thr=N-methyl-Threonine,
PyBOP=Benzotriazole-1-yl-oxy-tris-pyrrolidino-phosphonium hexafluorophosphate,
SPPS=Solid Phase Peptide Synthesis,
sc=subcutaneous,
TMS=Trimethylsilyl,
TIPS=Triisopropylsilane,
TFA=Trifluoroacetic acid,
TBTU=2-(1H-benzotriazole-1-yl)-1,1,3,3-tetramethylaminium tetrafluoroborate,
Trt=Trityl group.

### DETAILED DESCRIPTION OF THE INVENTION

The term ‘natural amino acids’ indicates all those twenty amino acids, which are present in nature.

The term ‘unnatural amino acids’ or ‘non-natural amino acids’ represents either replacement of L-amino acids with corresponding D-amino acids such as replacement of L-Ala with D-Ala, and the like or suitable modifications of the L or D amino acids, amino alkyl acids, either by
α-alkylation such as substitution of Ala with α-methyl Ala (Aib), replacement of Phe with α-methyl-Phe; or
substitution on the side chain of amino acid such as substitution of aromatic amino acid side chain with halogen, (C₁-C₃)alkyl, aryl groups, more specifically the replacement of Phe with 2 & 6-halo Phe;
   Unless otherwise indicated, the term ‘amino acid’ as employed herein alone or as part of another group includes, without limitation, an amino group and a carboxyl group linked to the same carbon, referred to as ‘α’ carbon.

The absolute ‘S’ configuration at the ‘α’ carbon is commonly referred to as the ‘L’ or natural configuration. The ‘R’ configuration at the ‘α’ carbon is commonly referred to as the ‘D’ amino acid. In the case where both the ‘α-substituents’ is equal, such as hydrogen or methyl, the amino acids are Gly or Aib and are not chiral.

The term ‘receptor antagonist’ refers to compounds that inhibit the activation of receptor and generation of secondary messenger such as cyclic AMP either by competitive or non-competitive binding.

The term ‘Glucagon receptor antagonist’ refers to compounds that inhibit activation of glucagon receptor.

The term ‘GLP-1 receptor modulator or agonist’ refers to a compound that acts at the GLP-1 receptor to alter its ability to regulate downstream signaling events, such as cAMP production and insulin release. Example of receptor modulators includes agonist, partial agonist, inverse agonist and allosteric potentiators.

Accordingly, the present invention relates to compounds of the general Formula (I),

A-B—C-D (I)

and their tautomeric forms, pharmaceutically acceptable salts and pharmaceutical composition containing them, wherein
‘A’ represents a naturally or unnaturally occurring amino acid selected from Val (V), Lys (K), Leu (L), Ala (A), or Aib, or a dipeptide consisting of two amino acids selected from Val, Ala, Aib & Gly, operably linked to each other, such as Val-Val, Val-Ala, Val-Aib, Val-Gly, Gly-Val, Aib-Val, or Ala-Val;
‘B’ is defined by general Formula (II),

Z₁—Z₂—Z₃—Z₄—Z₅—Z₆—Z₇—Z₈—Z₉ (II)

wherein Z₁-Z₉ represents either the first N-terminal 1-9 residues of GLP-1 or glucagon peptide, or Z₁-Z₉ represents suitably modified N-terminal 1-9 residues, wherein one or more of these residues are independently suitably replaced with homologues unnatural/modified amino acids, wherein when any of Z₁-Z₉ represents homologues unnatural/modified amino acids, then Z₁ represents Histidine (His; H) or α-methyl-Histidine (αMe-His); Each of Z₂ & Z₄ independently represents a naturally or unnaturally occurring amino acid selected from Serine (Ser; S), Glycine (Gly; G), Alanine (Ala; A), α-methyl proline (αMe-Pro), α-amino-isobutyric acid (Aib), 1-amino cyclopropane carboxylic acid (AC₃C), or 1-amino-cyclopentanecarboxylic acid (AC₅C);

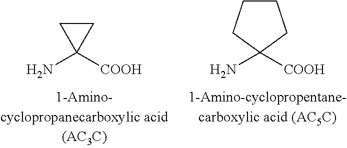

each of Z₃ and Z₉ independently represents a naturally or unnaturally occurring amino acid having an amino acid side chain comprising an acidic or amide group; each of Z₅, Z₇ & Z₈ independently represents a naturally or non-naturally occurring amino acid comprising a hydroxyl side chain; Z₆ represents a naturally or unnaturally occurring amino acid having a disubstituted alpha carbon having two side chains, wherein each of them may independently be an optionally substituted alkyl or aryl or an aralkyl group, wherein the substituents on the alkyl or aryl or aralkyl group is selected from one or more alkyl groups or one or more halo groups;
‘C’ represents a dipeptide of Formula III,

Z₁₀—Z₁₁₁) (III)

wherein Z₁₀ represents 2′-ethyl-4′-methoxy-biphenylalanine [Bip(OMe)] or α-methylated Bip(OMe) [αMe-Bip(OMe)];

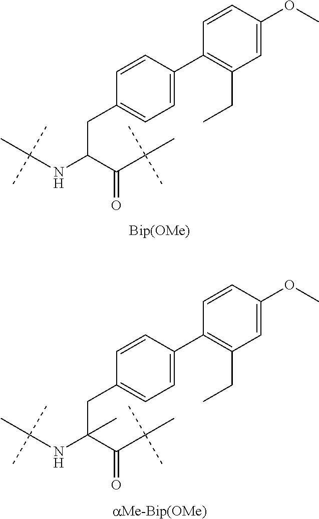

Z₁₁ represents 2-Amino-5-phenyl-pentanoic acid (APPA) or α-methylated APPA (αMe-APPA); and

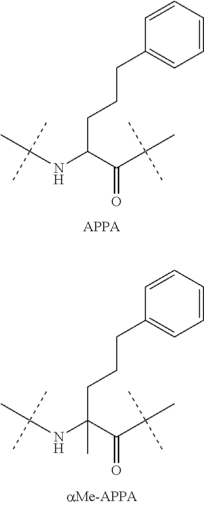

‘D’ represents a suitable C-terminus of peptidomimetics or a suitable amino acid. In an embodiment there are disclosed compound of Formula (I), wherein Z₃ represents amino acids selected from Glutamine (Gln; Q), Glutamic acid (Glu; Q), Aspartic acid (Asp; D), alpha-methyl-Glutamine (αMe-Gln), alpha-methyl-Glutamic acid (αMe-Glu), alpha-methyl-Aspartic acid (αMe-Asp), N-methyl-Glutamine (NMe-Gln), N-methyl-Glutamic acid (NMe-Glu), or N-methyl-Aspartic acid (NMe-Asp), while all other symbols are as defined earlier.
In another embodiment there are provided compounds of Formula (I), wherein Z₉ represents amino acids selected from Glutamine (Gln; Q), Glutamic acid (Glu; Q), Aspartic acid (Asp; D), alpha-methyl-Glutamine (αMe-Gln), alpha-methyl-Glutamic acid (αMe-Glu), alpha-methyl-Aspartic acid (αMe-Asp), N-methyl-Glutamine (NMe-Gln), N-methyl-Glutamic acid (NMe-Glu), or N-methyl-Aspartic acid (NMe-Asp), while all other symbols are as defined earlier.
In yet another embodiment there are provided compounds of Formula (I), wherein Z₅ is selected from Threonine (Thr; T), Serine (Ser; S), alpha-methyl-Threonine (αMe-Thr), alpha-methyl-Serine (αMe-Ser), Serine(OMe)-OH (Ser(OMe)), alpha-methyl-Serine(OMe)-OH (αMe-Ser(OMe)), N-methyl-Threonine (NMe-Thr), N-methyl-Serine (NMe-Ser), or 1-amino cyclopropane carboxylic acid (AC₃C) and all other symbols are as defined earlier.

In still another embodiment there are provided compounds of Formula (I), wherein Z₇ is selected from Threonine (Thr; T), Serine (Ser; S), alpha-methyl-Threonine (αMe-Thr), alpha-methyl-Serine (αMe-Ser), Serine(OMe)-OH (Ser(OMe)), alpha-methyl-Serine(OMe)-OH (αMe-Ser(OMe)), N-methyl-Threonine (NMe-Thr), N-methyl-Serine (NMe-Ser), or 1-amino cyclopropane carboxylic acid (AC₃C) and all other symbols are as defined earlier.

In another embodiment there are provided compounds of Formula (I), wherein Z₈ is selected from Threonine (Thr; T), Serine (Ser; S), alpha-methyl-Threonine (αMe-Thr), alpha-methyl-Serine (αMe-Ser), Serine(OMe)-OH (Ser(OMe)), alpha-methyl-Serine(OMe)-OH (αMe-Ser(OMe)), N-methyl-Threonine (NMe-Thr), N-methyl-Serine (NMe-Ser), or 1-amino cyclopropane carboxylic acid (AC₃C) and all other symbols are as defined earlier.

In yet another embodiment there are provided compounds of Formula (I), wherein Z₆ represents phenylalanine (Phe; F), alpha-methyl-phenylalanine (-αMe-Phe-), alpha-methyl-2-fluorophenylalanine (-αMe-2F-Phe-) or alpha-methyl-2,6-difluorophenylalanine (-αMe-2,6-F-Phe-), or 2-fluorophenylalanine (−2F-Phe-) and all other symbols are as defined earlier.

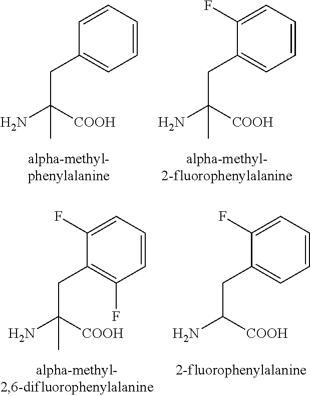

In another embodiment there are provided compounds of Formula (I), wherein ‘D’ represents the C-terminus of peptidomimetics selected from —COOH, —CONH₂, or —CH₂OH and all other symbols are as defined earlier.

In still another embodiment there are provided compounds of Formula (I), wherein ‘D’ represents a suitable amino acid selected from Val (V), Lys (K), Leu (L), Ala (A), or Aib and all other symbols are defined earlier.

In another embodiment there are provided compounds of Formula (I), wherein ‘A’ represents a naturally or unnaturally occurring amino acid selected from Val (V), Lys (K), Leu (L), Ala (A), or Aib or a dipeptide consisting of two amino acids selected from Val, Ala, Aib & Gly, operably linked to each other, such as Val-Val, Val-Ala, Val-Aib, Val-Gly, Gly-Val, Aib-Val, or Ala-Val;

‘B’ represents the first N-terminal 1-9 residues of GLP-1 or glucagon peptide, defined by general Formula II,

Z₁—Z₂—Z₃—Z₄—Z₅—Z₆—Z₇—Z₈—Z₉ (II)

wherein, Z₁-to-Z₉ are optionally replaced with homologues unnatural amino acids to improve its metabolic stability, wherein Z₁ represents Histidine (His; H) or α-methyl-Histidine (αMe-His); each of Z₂ & Z₄ independently represents a naturally or unnaturally occurring amino acid selected from Serine (Ser; S), Glycine (Gly; G), Alanine (Ala; A), α-methyl proline (αMe-Pro), α-amino-isobutyric acid (Aib), 1-amino cyclopropane carboxylic acid (AC₃C), or 1-amino-cyclopentanecarboxylic acid (AC₅C);

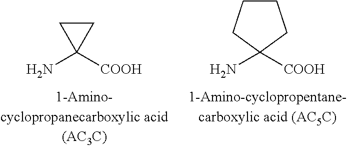

each of Z₃ and Z₉ independently represents a naturally or unnaturally occurring amino acid selected from Glutamine (Gln; Q), Glutamic acid (Glu; Q), Aspartic acid (Asp; D), alpha-methyl-Glutamine (αMe-Gln), alpha-methyl-Glutamic acid (αMe-Glu), alpha-methyl-Aspartic acid (αMe-Asp), N-methyl-Glutamine (NMe-Gln), N-methyl-Glutamic acid (NMe-Glu), N-methyl-Aspartic acid (NMe-Asp); each of Z₅, Z₇ & Z₈ independently represents a naturally or non-naturally occurring amino acid selected from Threonine (Thr; T), Serine (Ser; S), alpha-methyl-Threonine (αMe-Thr), alpha-methyl-Serine (αMe-Ser), Serine(OMe)-OH (Ser(OMe)), alpha-methyl-Serine(OMe)—OH (αMe-Ser(OMe)), N-methyl-Threonine (NMe-Thr), N-methyl-Serine (NMe-Ser), or 1-amino cyclopropane carboxylic acid (AC₃C); Z₆ represents phenylalanine (Phe; F), alpha-methyl-phenylalanine (-αMe-Phe-), alpha-methyl-2-fluorophenylalanine (-αMe-2F-Phe-) or alpha-methyl-2,6-difluorophenylalanine (-αMe-2,6-F-Phe-), or 2-fluorophenylalanine (-2F-Phe-);

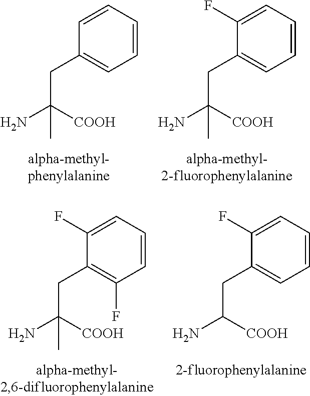

‘C’ represents a dipeptide of Formula III,

Z₁₀—Z₁₁ (III)

wherein Z₁₀ represents 2′-ethyl-4′-methoxy-biphenylalanine [Bip(OMe)] or α-methylated Bip(OMe) [αMe-Bip(OMe)];

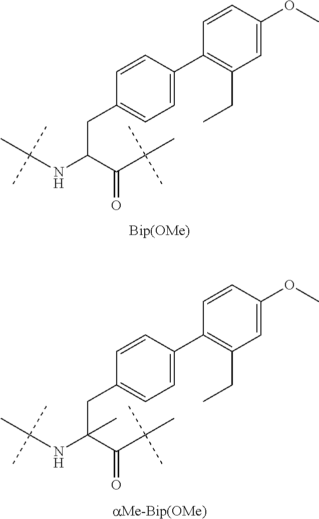

Z₁₁ represents 2-Amino-5-phenyl-pentanoic acid (APPA) or α-methylated APPA (αMe-APPA); and

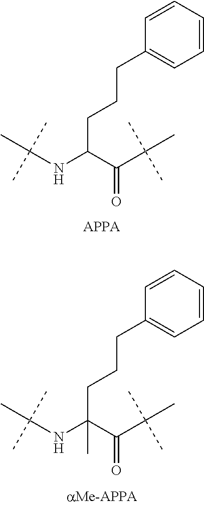

‘D’ represents groups selected from —COOH, —CONH₂, —CH₂OH or suitable amino acids selected from Val (V), Lys (K), Leu (L), Ala (A), or Aib.
The various groups, radicals and substituents used anywhere in the specification are described in the following paragraphs.
“alkyl” group is selected from linear or branched containing one to three carbons selected from methyl, ethyl, propyl, isopropyl, and the like.
“aryl” group is selected from phenyl, napthyl, indanyl, fluorenyl or biphenyl, groups; the heteroaryl group is selected from pyridyl, thienyl, furyl, imidazolyl, benzofuranyl groups.
“aralkyl” group used either alone or in combination with other radicals, is selected from groups containing aryl group, as define above, attached directly radical to an alkyl radical, as defined above, more preferably selected from benzyl, phenylethyl, and the like.

In another aspect of the invention there are provided processes for the preparation of compounds represented by the general Formula (I) as described below.

### Preparation of the Peptidomimetics:

Several synthetic routes can be employed to prepare the peptidomimetics of the present invention well known to one skilled in the art of peptide synthesis. The peptidomimetics of Formula (I), where all symbols are as defined earlier can be synthesized using the methods described below, together with conventional techniques known to those skilled in the art of peptide synthesis, or variations thereon as appreciated by those skilled in the art. Referred methods include, but not limited to those described below.

The peptidomimetics may be produced by chemical synthesis using suitable variations of various solid-phase techniques generally known, such as those described in G. Barany & R. B. Merrifield, “The peptides: Analysis, synthesis, Biology”; Volume 2—“Special methods in peptide synthesis, Part A”, pp. 3-284, E. Gross & J. Meienhofer, Eds., Academic Press, New York, 1980; and in J. M. Stewart and J. D. Young, “Solid-phase peptide synthesis” 2nd Ed., Pierce chemical Co., Rockford, Ill., 1984.

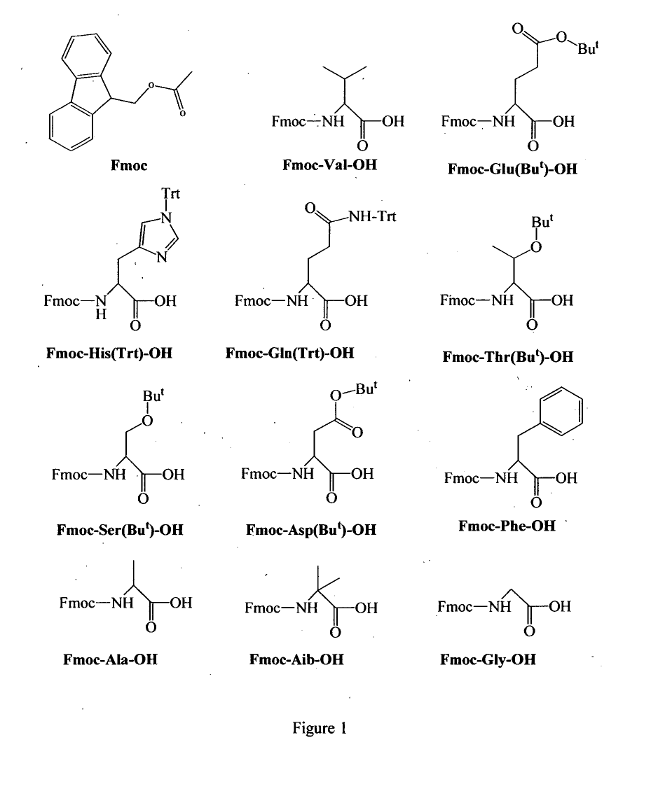

In general, the strategy for preparing the peptidomimetics of this invention is based on the use of Fmoc-based SPPS approach, wherein Fmoc (9-Fluorenyl-methyl-methyloxycarbonyl) group is used for temporary protection of the α-amino group in combination with the acid labile protecting groups, such as t-butyloxy carbonyl (Boc), tert-butyl (Bu^{t}), Trityl (Trt) groups (FIG. 1), for temporary protection of the amino acid side chains (See, for example E. Atherton & R. C. Sheppard, “The Fluorenylmethoxycarbonyl amino protecting group”, in “The peptides: Analysis, synthesis, Biology”; Volume 9—“Special methods in peptide synthesis, Part C”, pp. 1-38, S. Undenfriend & J. Meienhofer, Eds., Academic Press, San Diego, 1987).

The peptidomimetics can be synthesized in a stepwise manner on an insoluble polymer support (resin), starting form the C-terminus of the peptide. In an embodiment, the synthesis may be initiated by appending the C-terminal amino acid of the peptide to the resin through formation of an amide, ester or ether linkage. This allows the eventual release of the resulting peptide as a C-terminal amide, carboxylic acid or alcohol, respectively.

In the Fmoc-based SPPS, the C-terminal amino acid and all other amino acids used in the synthesis are required to have their α-amino groups and side chain functionalities (if present) differentially protected (orthogonal protection), such that the α-amino protecting group may be selectively removed during the synthesis, using a suitable base, such as 20% piperidine solution, without any premature cleavage of peptide from resin or deprotection of side chain protecting groups, usually protected with the acid labile protecting groups.

The coupling of an amino acid may be performed by activation of its carboxyl group as an active ester and reaction thereof with unblocked α-amino group of the N-terminal amino acid appended to the resin. After every coupling and deprotection, peptidyl-resin may be washed with the excess of solvents, such as DMF, DCM and diethyl ether. The sequence of α-amino group deprotection and coupling may be repeated until the desired peptide sequence is assembled (Scheme 1). The peptide may then be cleaved from the resin with concomitant deprotection of the side chain functionalities, using an appropriate cleavage mixture, usually in the presence of appropriate scavengers to limit side reactions. The resulting peptide may be finally purified by reverse phase HPLC.

The synthesis of the peptidyl-resins required as precursors to the final peptides may be obtained from commercially available cross-linked polystyrene polymer resins (Novabiochem, San Diego, Calif.). For example, Fmoc-PAL-PEG-PS resin, 4-(2′,4′-dimethoxyphenyl-Fmoc-aminomethyl)-phenoxyacetyl-p-methyl benzhydrylamine resin (Fmoc-Rink amide MBHA resin), or 2-chloro-Trityl-chloride resin or p-benzyloxybenzyl alcohol resin (HMP resin) to which the C-terminal amino acid may or may not be already attached. If the C-terminal amino acid is not attached, its attachment may be achieved by HOBt active ester of the Fmoc-protected amino acid formed by its reaction with DIPCDI. In case of 2-Chloro-trityl resin, the coupling of first Fmoc-protected amino acid may be achieved using DIPEA. For the assembly of next amino acid, N-terminal protection of peptidyl resin may be selectively deprotected using a solution of 10-20% piperidine solution. After every coupling and deprotection, excess of amino acids and coupling reagents may be removed by washing with DMF, DCM and ether. The coupling of subsequent amino acids can be accomplished using HOBt or HOAT active esters produced from DIPCDI/HOBt or DIPCDI/HOAT, respectively. In case of some difficult coupling, especially coupling of those amino acids, which are hydrophobic or amino acids with bulky side chain protection; complete coupling can be achieved using a combination of highly efficient coupling agents such as HBTU, PyBOP or TBTU, with additives such as DIPEA.

The synthesis of the peptidomimetics described herein can be carried out by using batchwise or continuous flow peptide synthesis apparatus, such as CS-Bio or AAPPTEC peptide synthesizer, utilizing the Fmoc/t-butyl protection strategy. The non-natural non-commercial amino acids present at different position may be incorporated into the peptide chain, using one or more methods known in the art. In one approach, Fmoc-protected non-natural amino acid was prepared in solution, using appropriate literature procedures. For example, the Fmoc-protected Bip analogs, described above, were prepared using modified Suzuki cross coupling method, as known in literature (Kotha, S., et al., Tetrahedron 2002, 58, 9633). The Fmoc-protected α-methylated amino acids were prepared using asymmetric Strecker synthesis (Boesten, W. H. J., et al., Org. Lett., 2001, 3(8), 1121). The resulting derivative was then used in the step-wise synthesis of the peptide. Alternatively, the required non-natural amino acid was built on the resin directly using synthetic organic chemistry procedures and a linear peptide chain were built.

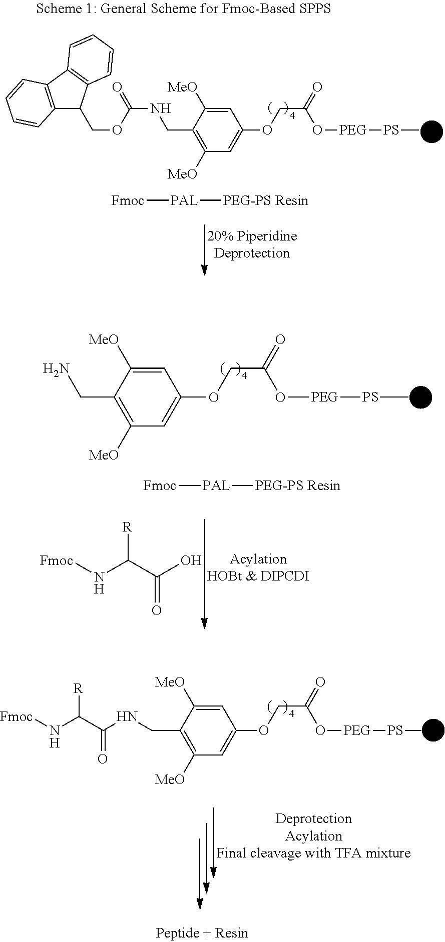

The peptide-resin precursors for their respective peptidomimetics may be cleaved and deprotected using suitable variations of any of the standard cleavage procedures described in the literature (King, D. S., et al., Int. J. Peptide Protein Res., 1990, 36, 255). In particular, the use of TFA cleavage mixture in the presence of water and TIPS as scavengers may be used. In general, the peptidyl-resin may be incubated in TFA/Water/TIPS (94:3:3; V:V:V; 10 ml/100 mg of peptidyl resin) for 1.5-2 hrs at room temperature. The cleaved resin may be filtered off and the TFA solution may be concentrated or dried under reduced pressure. The resulting crude peptide may be either precipitated or washed with Et₂O or re-dissolved directly into DMF or 50% aqueous acetic acid for purification by preparative HPLC.

The peptidomimetics with the desired purity can be obtained by purification using preparative HPLC. The solution of crude peptide may be injected into a semi-Prep column (Luna 10μ; C₁₈; 100 A°), dimension 250×50 mm and eluted with a linear gradient of ACN in water, both buffered with 0.1% TFA, using a flow rate of 15-50 ml/min with effluent monitoring by PDA detector at 220 nm. The structures of the purified peptidomimetics can be confirmed by Electrospray Mass Spectroscopy (ES-MS) analysis.

The peptides may be prepared and isolated as trifluoro-acetate salt, with TFA as a counter ion, after the Prep-HPLC purification. Alternatively, the peptides may be subjected to desalting by passing through a suitable ion exchange resin bed, for example through anion-exchange resin Dowex SBR P(Cl) or an equivalent basic anion-exchange resin. The TFA counter ions may also be replaced with acetate ions by passing through a suitable ion-exchange resin, eluted with dilute acetic acid solution. For the preparation of the hydrochloride salt of peptides, in the last stage of the manufacturing, selected peptides, with the acetate salt may be treated with 4 M HCl. The resulting solution may be filtered through a membrane filter (0.2 μm) and subsequently lyophilized to yield the white to off-white HCl salt. Following similar techniques and/or such suitable modifications, which are well within the scope of persons skilled in the art, other suitable pharmaceutically acceptable salts of the peptidomimetics of the present invention may be prepared.

### General Method of Preparation of Peptidomimetics, Using SPPS Approach:

### Assembly of Peptidomimetics on Resin:

Sufficient quantity (50-100 mg) of Fmoc-PAL-PEG-PS resin or Fmoc-Rink amide MBHA resin, loading: 0.5-0.6 mmol/g was swelled in DMF (10-20 ml/100 mg of resin) for 2-10 minutes. The Fmoc-group on resin was then removed by incubation of resin with 10-20% piperidine in DMF (10-30 ml/100 mg of resin), for 10-30 minutes. The deprotected resin was filtered and washed with excess of DMF, DCM and ether (50 ml×4). The washed resin was incubated in a freshly distilled DMF (1 ml/100 mg of resin), under nitrogen atmosphere for 5 minutes. A 0.5 M solution of first Fmoc-protected amino acid (1-3 eq.), pre-activated with HOBt (1-3 eq.) and DIPCDI (1-2 eq.) in DMF was added to the resin and the resin was then shaken for 1-3 hrs, under nitrogen atmosphere. The completion of coupling was monitored using a qualitative ninhydrin test. After the coupling of first amino acid, the resin was washed with DMF, DCM and Diethyl ether (50 ml×4). For the coupling of the next amino acid, the Fmoc-protection on first amino acid, coupled with resin was deprotected first using 20% piperidine solution, followed by the coupling of the Fmoc-protected second amino acid using suitable coupling agents, and as described above. The repeated cycles of deprotection, washing, coupling and washing were performed until the desired peptide chain was assembled on resin, as per general Scheme 1 above.

Finally, the Fmoc-protected peptidyl-resin prepared above was deprotected by 20% piperidine treatment as described above and the peptidyl-resins were washed with DMF, DCM and Diethyl ether (50 ml×4). The resin containing desired peptide was dried under nitrogen pressure for 10-15 minutes and subjected to cleavage/deprotection.

Using the above protocol and suitable variations thereof, which are within the scope of a person skilled in the art, the peptidomimetics designed in the present invention were prepared, using Fmoc-SPPS approach. Furthermore, the resin bound peptidomimetics were cleaved and deprotected, purified and characterized using the following protocol.

### Cleavage and Deprotection:

The desired peptidomimetics were cleaved and deprotected from their respective peptidyl-resins by treatment with TFA cleavage mixture as follows. A solution of TFA/Water/Triisopropylsilane (95:2.5:2.5) (10 ml/100 mg of peptidyl-resin) was added to peptidyl-resins and the mixture was kept at room temperature with occasional stirring. The resin was filtered, washed with a cleavage mixture and the combined filtrate was evaporated to dryness. The residue obtained was dissolved in 10 ml of water and the aqueous layer was extracted 3 times with ether (20 ml each) and finally, the aqueous layer was freeze-dried. In general, the purity of crude peptidomimetics obtained after freeze-drying was found to be in range of 60-70%, with 50-60% yield/recovery. Finally, the crude peptidomimetics was purified by preparative HPLC as follows:

### Preparative HPLC Purification of the Crude Peptidomimetics:

The Preparative HPLC was carried out on a Shimadzu LC-8A liquid chromatograph. A solution of crude peptide dissolved in DMF or water was injected into a semi-Prep column (Luna 10μ; C₁₈; 100 A°), dimension 250×50 mm and eluted with a linear gradient of ACN in water, both buffered with 0.1% TFA, using a flow rate of 15-50 ml/min, with effluent monitoring by PDA detector at 220 nm. A typical gradient of 20% to 70% of water-ACN mixture, buffered with 0.1% TFA was used, over a period of 50 minutes, with 1% gradient change per minute. The desired product eluted were collected in a single 10-20 ml fraction and the pure peptidomimetics were obtained as amorphous white powders by lyophilization of respective HPLC fractions. In general, after the Prep-HPLC purification, the overall recovery was found to be in the range of 40-50% (yield).

### HPLC Analysis of the Purified Peptidomimetics

After purification by preparative HPLC as described above, each peptide was analyzed by analytical RP-HPLC on a Shimadzu LC-10AD analytical HPLC system. For analytic HPLC analysis of peptidomimetics, Luna 5μ; C₁₈; 100 A°, dimension 250×4.6 mm column was used, with a linear gradient of 0.1% TFA and ACN buffer and the acquisition of chromatogram was carried out at 220 nm, using a PDA detector. In general, the purity of pure peptidomimetics obtained after Prep-HPLC purification was found to be >95%.

Characterization by Mass Spectrometry

Each peptide was characterized by electrospray ionisation mass spectrometry (ESI-MS), either in flow injection or LC/MS mode. Triple quadrupole mass spectrometers (API-3000 (MDS-SCIES, Canada) was used in all analyses in positive and negative ion electrospray mode. The full scan data was acquired over the mass range of quadrupole, operated at unit resolution. In all cases, the experimentally measured molecular weight was within 0.5 Daltons of the calculated monoisotopic molecular weight. Quantification of the mass chromatogram was done using Analyst 1.4.1 software. Using the above described protocol, all the crude/pure peptidomimetics were characterized by mass spectroscopy and in general, observed mass of peptidomimetic was found to be in agreement with calculated/theoretical mass, which confirms successful synthesis of desired peptidomimetics.

### Representative Example of Automated Solid Phase Synthesis of Peptide Sequence ID No. 159 (H₂N-V-H-Aib-Q-G-T-F-T-(αMe-Ser)-D-(αMe-Bip(OMe))-(αMe-APPA)-CONH₂).

The linear peptide chain, H₂N-V-H-Aib-Q-G-T-F-T-(αMe-Ser)-D-(αMe-Bip(OMe))-(αMe-APPA)-PAL-PEG-PS was assembled on an automated CS-Bio 536 PepSynthesiser™ using Fmoc solid phase peptide synthesis (SPPS) approach (Scheme 2). The Fmoc amino acids and the 2-(1H-Benzotriazol-1-yl)-1,1,3,3-tetramethyluroniumtetrafluoroborate (TBTU) were packed together in vials and positioned in the amino acid module of the synthesizer. A stock solution of diisopropylethylamine (DIPEA; 0.9 M) and DMF were stored in reagent bottles, under dry nitrogen atmosphere. The resin, Fmoc-PAL-PEG-PS (0.38 mmol/g; 1 g) was dried over P₂O₅, in vacuo (1 hr) and swollen in freshly distilled DMF (5 mL). The swollen resin was slurry packed into a glass column and positioned in the synthesizer. All the synthetic cycles were carried out at a flow rate of 5 mL min⁻¹, Table 1. The resin was washed with a freshly distilled DMF for 10 minutes. The deprotection of Fmoc group was performed with 20% piperidine in DMF for 10 minutes and the deprotection was monitored by UV detection of the column effluent at 304 nm.

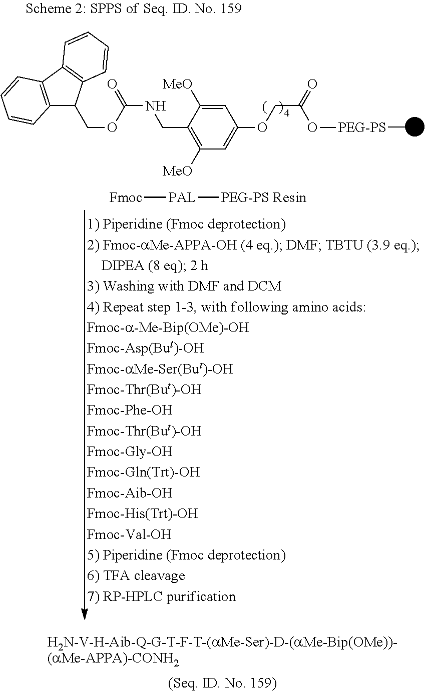

The excess piperidine was removed by three auxiliary wash cycles and a distilled DMF wash cycle, with each cycle of 15 minutes. The amino group was treated with Fmoc-amino acid (4 equivalent), preactivated with TBTU (3.9 equivalent) in the presence of DIPEA (8 equivalent) and recycled for 120 minutes. The excess amino acid and soluble by-products were removed from the column and loop by four auxiliary wash cycles and distilled DMF wash cycles, with each cycle of 10 minutes. Furthermore, the synthetic cycles (deprotection, wash, acylation and wash) were repeated for the complete assembly of linear peptide. The final deprotection cycle was performed with 20% piperidine in DMF for 15 minutes to remove the terminal Fmoc group, followed by wash cycle (10×4 minutes). The completed peptide-resin was filtered through a sintered glass filter, washed three times successively with DMF, DCM, methanol, DMF and diethyl ether (100 mL each). The Peptide-resin was dried in vacuo over P₂O₅ (2 hr) and stored at −20° C. The Ninhydrin resin test was carried out to check the N-terminal free amino group of resin bound peptide. The appearance of a blue-purple colouration of the solution and the resin beads indicates the presence of free amino group on resin bound peptide and was considered to be a positive test.

TABLE 1
Automated cycles for solid phase peptide synthesis

| | | | Number of | Time |
|---|---|---|---|---|
| Step | Function | Reagent/Solvent | cycles | (Minute) |
| 1 | Wash | Dimethylformamide (DMF) | 1 | 10 |
| 2 | Deprotection | 20% piperidine in DMF | 2 | 15 |
| 3 | Wash | DMF | 3 | 15 |
| 4 | Acylation | Amino acid; TBTU and | Recycle | 120 |
| | | diisopropylethylamine | | |
| | | (in DMF) | | |
| 5 | Wash | Dimethylformamide | 4 | 10 |

A small-scale cleavage was carried out to assess the purity of resin bound peptide. The dried Peptide-resin (ca 10-mg) was treated with a mixture (1 mL) of TFA, water, and triisopropylsilane (95:2.5:2.5 v/v), for 90 minutes at room temperature with gentle occasional swirling. The resin was filtered, washed thoroughly with neat TFA (1 mL) and the entire filtrate was evaporated under reduced pressure. The residual TFA was azeotroped three times with diethyl ether (2 mL). The residue obtained was suspended in distilled water (2 mL) and the aqueous layer was extracted three times with diethyl ether (3 mL). The aqueous layer was separated and freeze-dried to yield the crude peptidomimetics (H₂N-V-H-Aib-Q-G-T-F-T-(αMe-Ser)-D-(Me-Bip(OMe))-(αMe-APPA)-CONH₂) as white powder. The lyophilized peptidomimetics H₂N-V-H-Aib-Q-G-T-F-T-(αMe-Ser)-D-(αMe-Bip(OMe))-(αMe-APPA)-CONH₂ was dissolved in 0.1% aqueous TFA (ca 1 mg/1 mL) and its purity was analyzed by analytical RP-HPLC and characterized by electrospray ionization mass spectrometry (ESI-MS). Percent purity: 60%; Percent yield: 55% (crude peptidomimetic). Furthermore, the crude peptidomimetic was purified by Prep-HPLC and lyophilized to get a white fluffy powder (purity after Prep-HPLC purification: 96%; yield: 49%). ESI-MS; Calcd. for H₂N-V-H-Aib-Q-G-T-F-T-(αMe-Ser)-D-(αMe-Bip(OMe))-(αMe-APPA)-CONH₂: 1574 (M⁺), 1596 (M+Na⁺) and 1612 (M+K⁺). Found (m/z): 1574 (M⁺), 1596 (M+Na⁺) and 1612 (M+K⁺).

Utilizing the synthetic methods described herein along with other commonly known techniques and suitable variations thereof, the following novel peptidomimetics were prepared. This list is indicative of the various groups of peptidomimetics, which are provided merely to be exemplary of the invention and do not limit the scope of the invention. Certain modifications and equivalents will be apparent to those skilled in the art and are intended to be included within the scope of the invention.

In Table 2-(1-ix), novel peptidomimetics of present invention are listed along with their corresponding Seq. ID. No.

TABLE 2 (i)
List of peptidomimetics prepared

| Seq. | |
|---|---|
| ID. No. | Sequence of Peptidomimetics |
| 5 | VHSQGTFTSD-(αMe-Bip(OMe))-(APPA) |
| 6 | VHAQGTFTSD-(αMe-Bip(OMe))-(APPA) |
| 7 | VH-Aib-QGTFTSD-(αMe-Bip(OMe))-(APPA) |
| 8 | VH-(αMe-Pro)-QGTFTSD-(αMe-Bip(OMe))-(APPA) |
| 9 | VH-(AC₅C)-QGTFTSD-(αMe-Bip(OMe))-(APPA) |
| 10 | VH-(AC₃C)-QGTFTSD-(αMe-Bip(OMe))-(APPA) |
| 11 | VHSEGTFTSD-(αMe-Bip(OMe))-(APPA) |
| 12 | VHAEGTFTSD-(αMe-Bip(OMe))-(APPA) |
| 13 | VH-Aib-EGTFTSD-(αMe-Bip(OMe))-(APPA) |
| 14 | VH-(αMe-Pro)-EGTFTSD-(αMe-Bip(OMe))-(APPA) |
| 15 | VH-(AC₅C)-EGTFTSD-(αMe-Bip(OMe))-(APPA) |
| 16 | VH-(AC₃C)-EGTFTSD-(αMe-Bip(OMe))-(APPA) |
| 17 | VHSQGT-(αMe-2FPhe)-TSD-(αMe-Bip(OMe))-(APPA) |
| 18 | VHAQGT-(αMe-2FPhe)-TSD-(αMe-Bip(OMe))-(APPA) |
| 19 | VH-Aib-QGT-(αMe-2FPhe)-TSD-(αMe-Bip(OMe))-(APPA) |
| 20 | VH-(αMe-Pro)-QGT-(αMe-2FPhe)-TSD-(αMe-Bip(OMe))-(APPA) |
| 21 | VHSEGT-(αMe-2FPhe)-TSD-(αMe-Bip(OMe))-(APPA) |
| 22 | VHAEGT-(αMe-2FPhe)-TSD-(αMe-Bip(OMe))-(APPA) |
| 23 | VH-Aib-EGT-(αMe-2FPhe)-TSD-(αMe-Bip(OMe))-(APPA) |
| 24 | VH-(αMe-Pro)-EGT-(αMe-2FPhe)-TSD-(αMe-Bip(OMe))-(APPA) |
| 25 | VHSQGT-(αMe-2,6-diF-Phe)-TSD-(αMe-Bip(OMe))-(APPA) |
| 26 | VHAQGT-(αMe-2,6-diFPhe)-TSD-(αMe-Bip(OMe))-(APPA) |
| 27 | VH-Aib-QGT-(αMe-2,6-diFPhe)-TSD-(αMe-Bip(OMe))-(APPA) |
| 28 | VH-(αMe-Pro)-QGT-(αMe-2,6-diFPhe)-TSD-(αMe-Bip(OMe))-(APPA) |
| 29 | VHSEGT-(αMe-2,6-diFPhe)-TSD-(αMe-Bip(OMe))-(APPA) |
| 30 | VHAEGT-(αMe-2,6-diFPhe)-TSD-(αMe-Bip(OMe))-(APPA) |
| 31 | VH-Aib-EGT-(αMe-2,6-diFPhe)-TSD-(αMe-Bip(OMe))-(APPA) |
| 32 | VH-(αMe-Pro)-EGT-(αMe-2,6-diFPhe)-TSD-(αMe-Bip(OMe))-(APPA) |

TABLE 2 (ii)
List of peptidomimetics prepared

| Seq. | |
|---|---|
| ID. No. | Sequence of Peptidomimetics |
| 33 | VHSQGTFT-(αMe-Ser)-D-(αMe-Bip(OMe))-(APPA) |
| 34 | VHAQGTFT-(αMe-Ser)-D-(αMe-Bip(OMe))-(APPA) |
| 35 | VH-Aib-QGTFT-(αMe-Ser)-D-(αMe-Bip(OMe))-(APPA) |
| 36 | VH-(αMe-Pro)-QGTFT-(αMe-Ser)-D-(αMe-Bip(OMe))-(APPA) |
| 37 | VHSEGTFT-(αMe-Ser)-D-(αMe-Bip(OMe))-(APPA) |
| 38 | VHAEGTFT-(αMe-Ser)-D-(αMe-Bip(OMe))-(APPA) |
| 39 | VH-Aib-EGTFT-(αMe-Ser)-D-(αMe-Bip(OMe))-(APPA) |
| 40 | VH-(αMe-Pro)-EGTFT-(αMe-Ser)-D-(αMe-Bip(OMe))-(APPA) |
| 41 | VHSQGT-(αMe-2FPhe)-T-(αMe-Ser)-D-(αMe-Bip(OMe))-(APPA) |
| 42 | VHAQGT-(αMe-2FPhe)-T-(αMe-Ser)-D-(αMe-Bip(OMe))-(APPA) |
| 43 | VH-Aib-QGT-(αMe-2FPhe)-T-(αMe-Ser)-D-(αMe-Bip(OMe))-(APPA) |
| 44 | VH-(αMe-Pro)-QGT-(αMe-2FPhe)-T-(αMe-Ser)-D-(αMe-Bip(OMe))-APPA) |
| 45 | VHSEGT-(αMe-2FPhe)-T-(αMe-Ser)-D-(αMe-Bip(OMe))-(APPA) |
| 46 | VHAEGT-(αMe-2FPhe)-T-(αMe-Ser)-D-(αMe-Bip(OMe))-(APPA) |
| 47 | VH-Aib-EGT-(αMe-2FPhe)-T-(αMe-Ser)-D-(αMe-Bip(OMe))-(APPA) |
| 48 | VH-(αMe-Pro)-EGT-(αMe-2FPhe)-T-(αMe-Ser)-D-(αMe-Bip(OMe))-APPA) |
| 49 | VHSQGT-(αMe-2,6-diF-Phe)-T-(αMe-Ser)-D-(αMe-Bip(OMe))-(APPA) |
| 50 | VHAQGT-(αMe-2,6-diFPhe)-T-(αMe-Ser)-D-(αMe-Bip(OMe))-(APPA) |
| 51 | VH-Aib-QGT-(αMe-2,6-diFPhe)-T-(αMe-Ser)-D-(αMe-Bip(OMe))-(APPA) |
| 52 | VH-(αMe-Pro)-QGT-(αMe-2,6-diFPhe)-T-(αMe-Ser)-D-(αMe-Bip(OMe))-(APPA) |
| 53 | VHSEGT-(αMe-2,6-diFPhe)-T-(αMe-Ser)-D-(αMe-Bip(OMe))-(APPA) |
| 54 | VHAEGT-(αMe-2,6-diFPhe)-T-(αMe-Ser)-D-(αMe-Bip(OMe))-(APPA) |
| 55 | VH-Aib-EGT-(αMe-2,6-diFPhe)-T-(αMe-Ser)-D-(αMe-Bip(OMe))-(APPA) |
| 56 | VH-(αMe-Pro)-EGT-(αMe-2,6-diFPhe)-T-(αMe-Ser)-D-(αMe-Bip(OMe))-(APPA) |

TABLE 2 (iii)
List of peptidomimetics prepared

| Seq. | |
|---|---|
| ID. No. | Sequence of Peptidomimetics |
| 57 | VHSQGTFTS-(αMe-Asp)-(αMe-Bip(OMe))-(APPA) |
| 58 | VHAQGTFTS-(αMe-Asp)-(αMe-Bip(OMe))-(APPA) |
| 59 | VH-Aib-QGTFTS-(αMe-Asp)-(αMe-Bip(OMe))-(APPA) |
| 60 | VH-(αMe-Pro)-QGTFTS-(αMe-Asp)-(αMe-Bip(OMe))-(APPA) |
| 61 | VHSEGTFTS-(αMe-Asp)-(αMe-Bip(OMe))-(APPA) |
| 62 | VHAEGTFTS-(αMe-Asp)-(αMe-Bip(OMe))-(APPA) |
| 63 | VH-Aib-EGTFTS-(αMe-Asp)-(αMe-Bip(OMe))-(APPA) |
| 64 | VH-(αMe-Pro)-EGTFTS-(αMe-Asp)-(αMe-Bip(OMe))-(APPA) |
| 65 | VHSQGT-(αMe-2FPhe)-TS-(αMe-Asp)-(αMe-Bip(OMe))-(APPA) |
| 66 | VHAQGT-(αMe-2FPhe)-TS-(αMe-Asp)-(αMe-Bip(OMe))-(APPA) |
| 67 | VH-Aib-QGT-(αMe-2FPhe)-TS-(αMe-Asp)-(αMe-Bip(OMe))-(APPA) |
| 68 | VH-(αMe-Pro)-QGT-(αMe-2FPhe)-TS-(αMe-Asp)-(αMe-Bip(OMe))-(APPA) |
| 69 | VHSEGT-(αMe-2FPhe)-TS-(αMe-Asp)-(αMe-Bip(OMe))-(APPA) |
| 70 | VHAEGT-(αMe-2FPhe)-TS-(αMe-Asp)-(αMe-Bip(OMe))-(APPA) |
| 71 | VH-Aib-EGT-(αMe-2FPhe)-TS-(αMe-Asp)-(αMe-Bip(OMe))-(APPA) |
| 72 | VH-(αMe-Pro)-EGT-(αMe-2FPhe)-TS-(αMe-Asp)-(αMe-Bip(OMe))-(APPA) |
| 73 | VHSQGT-(αMe-2,6-diF-Phe)-TS-(αMe-Asp)-(αMe-Bip(OMe))-(APPA) |
| 74 | VHAQGT-(αMe-2,6-diFPhe)-TS-(αMe-Asp)-(αMe-Bip(OMe))-(APPA) |
| 75 | VH-Aib-QGT-(αMe-2,6-diFPhe)-TS-(αMe-Asp)-(αMe-Bip(OMe))-(APPA) |
| 76 | VH-(αMe-Pro)-QGT-(αMe-2,6-diFPhe)-TS-(αMe-Asp)-(αMe-Bip(OMe))-(APPA) |
| 77 | VHSEGT-(αMe-2,6-diFPhe)-TS-(αMe-Asp)-(αMe-Bip(OMe))-(APPA) |
| 78 | VHAEGT-(αMe-2,6-diFPhe)-TS-(αMe-Asp)-(αMe-Bip(OMe))-(APPA) |
| 79 | VH-Aib-EGT-(αMe-2,6-diFPhe)-TS-(αMe-Asp)-(αMe-Bip(OMe))-(APPA) |
| 80 | VH-(αMe-Pro)-EGT-(αMe-2,6-diFPhe)-TS-(αMe-Asp)-(αMe-Bip(OMe))-(APPA) |

TABLE 2 (iv)
List of peptidomimetics prepared

| Seq. | |
|---|---|
| ID. No. | Sequence of Peptidomimetics |
| 81 | VHSQGTFTSD-Bip(OMe)-(αMe-APPA) |
| 82 | VHAQGTFTSD-Bip(OMe)-(αMe-APPA) |
| 83 | VH-Aib-QGTFTSD-Bip(OMe)-(αMe-APPA) |
| 84 | VH-(αMe-Pro)-QGTFTSD-Bip(OMe)-(αMe-APPA) |
| 85 | VH-(AC₅C)-QGTFTSD-Bip(OMe)-(αMe-APPA) |
| 86 | VH-(AC₃C)-QGTFTSD-Bip(OMe)-(αMe-APPA) |
| 87 | VHSEGTFTSD-Bip(OMe)-(αMe-APPA) |
| 88 | VHAEGTFTSD-Bip(OMe)-(αMe-APPA) |
| 89 | VH-Aib-EGTFTSD-Bip(OMe)-(αMe-APPA) |
| 90 | VH-(αMe-Pro)-EGTFTSD-Bip(OMe)-(αMe-APPA) |
| 91 | VHSQGT-(αMe-2FPhe)-TSD-Bip(OMe)-(αMe-APPA) |
| 92 | VHAQGT-(αMe-2FPhe)-TSD-Bip(OMe)-(αMe-APPA) |
| 93 | VH-Aib-QGT-(αMe-2FPhe)-TSD-Bip(OMe)-(αMe-APPA) |
| 94 | VH-(αMe-Pro)-QGT-(αMe-2FPhe)-TSD-Bip(OMe)-(αMe-APPA) |
| 95 | VHSEGT-(αMe-2FPhe)-TSD-Bip(OMe)-(αMe-APPA) |
| 96 | VHAEGT-(αMe-2FPhe)-TSD-Bip(OMe)-(αMe-APPA) |
| 97 | VH-Aib-EGT-(αMe-2FPhe)-TSD-Bip(OMe)-(αMe-APPA) |
| 98 | VH-(αMe-Pro)-EGT-(αMe-2FPhe)-TSD-Bip(OMe)-(αMe-APPA) |
| 99 | VHSQGT-(αMe-2,6-diF-Phe)-TSD-Bip(OMe)-(αMe-APPA) |
| 100 | VHAQGT-(αMe-2,6-diFPhe)-TSD-Bip(OMe)-(αMe-APPA) |
| 101 | VH-Aib-QGT-(αMe-2,6-diFPhe)-TSD-Bip(OMe)-(αMe-APPA) |
| 102 | VH-(αMe-Pro)-QGT-(αMe-2,6-diFPhe)-TSD-Bip(OMe)-(αMe-APPA) |
| 103 | VHSEGT-(αMe-2,6-diFPhe)-TSD-Bip(OMe)-(αMe-APPA) |
| 104 | VHAEGT-(αMe-2,6-diFPhe)-TSD-Bip(OMe)-(αMe-APPA) |
| 105 | VH-Aib-EGT-(αMe-2,6-diFPhe)-TSD-Bip(OMe)-(αMe-APPA) |
| 106 | VH-(αMe-Pro)-EGT-(αMe-2,6-diFPhe)-TSD-Bip(OMe)-(αMe-APPA) |

TABLE 2 (v)
List of peptidomimetics prepared

| Seq. | |
|---|---|
| ID. No. | Sequence of Peptidomimetics |
| 107 | VHSQGTFT-(αMe-Ser)-(αMe-Asp)-Bip(OMe)-(αMe-APPA) |
| 108 | VHAQGTFT-(αMe-Ser)-(αMe-Asp)-Bip(OMe)-(αMe-APPA) |
| 109 | VH-Aib-QGTFT-(αMe-Ser)-(αMe-Asp)-Bip(OMe)-(αMe-APPA) |
| 110 | VH-(αMe-Pro)-QGTFT-(αMe-Ser)-(αMe-Asp)-Bip(OMe)-(αMe-APPA) |
| 111 | VHSEGTFT-(αMe-Ser)-(αMe-Asp)-Bip(OMe)-(αMe-APPA) |
| 112 | VHAEGTFT-(αMe-Ser)-(αMe-Asp)-Bip(OMe)-(αMe-APPA) |
| 113 | VH-Aib-EGTFT-(αMe-Ser)-(αMe-Asp)-Bip(OMe)-(αMe-APPA) |
| 114 | VH-(αMe-Pro)-EGTFT-(αMe-Ser)-(αMe-Asp)-Bip(OMe)-(αMe-APPA) |
| 115 | VHSQGT-(αMe-2FPhe)-T-(αMe-Ser)-(αMe-Asp)-Bip(OMe)-(αMe-APPA) |
| 116 | VHAQGT-(αMe-2FPhe)-T-(αMe-Ser)-(αMe-Asp)-Bip(OMe)-(αMe-APPA) |
| 117 | VH-Aib-QGT-(αMe-2FPhe)-T-(αMe-Ser)-(αMe-Asp)-Bip(OMe)-(αMe-APPA) |
| 118 | VH-(αMe-Pro)-QGT-(αMe-2FPhe)-T-(αMe-Ser)-(αMe-Asp)-Bip(OMe)-(αMe-APPA) |
| 119 | VHSEGT-(αMe-2FPhe)-T-(αMe-Ser)-(αMe-Asp)-Bip(OMe)-(αMe-APPA) |
| 120 | VHAEGT-(αMe-2FPhe)-T-(αMe-Ser)-(αMe-Asp)-Bip(OMe)-(αMe-APPA) |
| 121 | VH-Aib-EGT-(αMe-2FPhe)-T-(αMe-Ser)-(αMe-Asp)-Bip(OMe)-(αMe-APPA) |
| 122 | VH-(αMe-Pro)-EGT-(αMe-2FPhe)-T-(αMe-Ser)-(αMe-Asp)-Bip(OMe)-(αMe-APPA) |
| 123 | VHSQGT-(αMe-2,6-diF-Phe)-T-(αMe-Ser)-(αMe-Asp)-Bip(OMe)-(αMe-APPA) |
| 124 | VHAQGT-(αMe-2,6-diFPhe)-T-(αMe-Ser)-(αMe-Asp)-Bip(OMe)-(αMe-APPA) |
| 125 | VH-Aib-QGT-(αMe-2,6-diFPhe)-T-(αMe-Ser)-(αMe-Asp)-Bip(OMe)-(αMe-APPA) |
| 126 | VH-(αMe-Pro)-QGT-(αMe-2,6-diFPhe)-T-(αMe-Ser)-(αMe-Asp)-Bip(OMe)-(αMe-APPA) |
| 127 | VHSEGT-(αMe-2,6-diFPhe)-T-(αMe-Ser)-(αMe-Asp)-Bip(OMe)-(αMe-APPA) |
| 128 | VHAEGT-(αMe-2,6-diFPhe)-T-(αMe-Ser)-(αMe-Asp)-Bip(OMe)-(αMe-APPA) |
| 129 | VH-Aib-EGT-(αMe-2,6-diFPhe)-T-(αMe-Ser)-(αMe-Asp)-Bip(OMe)-(αMe-APPA) |
| 130 | VH-(αMe-Pro)-EGT-(αMe-2,6-diFPhe)-T-(αMe-Ser)-(αMe-Asp)-Bip(OMe)-(αMe-APPA) |

TABLE 2 (vi)
List of peptidomimetics prepared

| Seq. | |
|---|---|
| ID. No. | Sequence of Peptidomimetics |
| 131 | VHSQGTFTSD-(αMe-Bip(OMe))-(αMe-APPA) |
| 132 | VHAQGTFTSD-(αMe-Bip(OMe))-(αMe-APPA) |
| 133 | VH-Aib-QGTFTSD-(αMe-Bip(OMe))-(αMe-APPA) |
| 134 | VH-(αMe-Pro)-QGTFTSD-(αMe-Bip(OMe))-(αMe-APPA) |
| 135 | VH-(AC₅C)-QGTFTSD-(αMe-Bip(OMe))-(αMe-APPA) |
| 136 | VH-(AC₃C)-QGTFTSD-(αMe-Bip(OMe))-(αMe-APPA) |
| 137 | VHSEGTFTSD-(αMe-Bip(OMe))-(αMe-APPA) |
| 138 | VHAEGTFTSD-(αMe-Bip(OMe))-(αMe-APPA) |
| 139 | VH-Aib-EGTFTSD-(αMe-Bip(OMe))-(αMe-APPA) |
| 140 | VH-(αMe-Pro)-EGTFTSD-(αMe-Bip(OMe))-(αMe-APPA) |
| 141 | VHSQGT-(αMe-2FPhe)-TSD-(αMe-Bip(OMe))-(αMe-APPA) |
| 142 | VHAQGT-(αMe-2FPhe)-TSD-(αMe-Bip(OMe))-(αMe-APPA) |
| 143 | VH-Aib-QGT-(αMe-2FPhe)-TSD-(αMe-Bip(OMe))-(αMe-APPA) |
| 144 | VH-(αMe-Pro)-QGT-(αMe-2FPhe)-TSD-(αMe-Bip(OMe))-(αMe-APPA) |
| 145 | VHSEGT-(αMe-2FPhe)-TSD-(αMe-Bip(OMe))-(αMe-APPA) |
| 146 | VHAEGT-(αMe-2FPhe)-TSD-(αMe-Bip(OMe))-(αMe-APPA) |
| 147 | VH-Aib-EGT-(αMe-2FPhe)-TSD-(αMe-Bip(OMe))-(αMe-APPA) |
| 148 | VH-(αMe-Pro)-EGT-(αMe-2FPhe)-TSD-(αMe-Bip(OMe))-(αMe-APPA) |
| 149 | VHSQGT-(αMe-2,6-diF-Phe)-TSD-(αMe-Bip(OMe))-(αMe-APPA) |
| 150 | VHAQGT-(αMe-2,6-diFPhe)-TSD-(αMe-Bip(OMe))-(αMe-APPA) |
| 151 | VH-Aib-QGT-(αMe-2,6-diFPhe)-TSD-(αMe-Bip(OMe))-(αMe-APPA) |
| 152 | VH-(αMe-Pro)-QGT-(αMe-2,6-diFPhe)-TSD-(αMe-Bip(OMe))-(αMe-APPA) |
| 153 | VHSEGT-(αMe-2,6-diFPhe)-TSD-(αMe-Bip(OMe))-(αMe-APPA) |
| 154 | VHAEGT-(αMe-2,6-diFPhe)-TSD-(αMe-Bip(OMe))-(αMe-APPA) |
| 155 | VH-Aib-EGT-(αMe-2,6-diFPhe)-TSD-(αMe-Bip(OMe))-(αMe-APPA) |
| 156 | VH-(αMe-Pro)-EGT-(αMe-2,6-diFPhe)-TSD-(αMe-Bip(OMe))-(αMe-APPA) |

TABLE 2 (vii)
List of peptidomimetics prepared

| Seq. | |
|---|---|
| ID. No. | Sequence of Peptidomimetics |
| 157 | VHSQGTFT-(αMe-Ser)-D-(αMe-Bip(OMe))-(αMe-APPA) |
| 158 | VHAQGTFT-(αMe-Ser)-D-(αMe-Bip(OMe))-(αMe-APPA) |
| 159 | VH-Aib-QGTFT-(αMe-Ser)-D-(αMe-Bip(OMe))-(αMe-APPA) |
| 160 | VH-(αMe-Pro)-QGTFT-(αMe-Ser)-D-(αMe-Bip(OMe))-(αMe-APPA) |
| 161 | VHSEGTFT-(αMe-Ser)-D-(αMe-Bip(OMe))-(αMe-APPA) |
| 162 | VHAEGTFT-(αMe-Ser)-D-(αMe-Bip(OMe))-(αMe-APPA) |
| 163 | VH-Aib-EGTFT-(αMe-Ser)-D-(αMe-Bip(OMe))-(αMe-APPA) |
| 164 | VH-(αMe-Pro)-EGTFT-(αMe-Ser)-D-(αMe-Bip(OMe))-(αMe-APPA) |
| 165 | VHSQGT-(αMe-2FPhe)-T-(αMe-Ser)-D-(αMe-Bip(OMe))-(αMe-APPA) |
| 166 | VHAQGT-(αMe-2FPhe)-T-(αMe-Ser)-D-(αMe-Bip(OMe))-(αMe-APPA) |
| 167 | VH-Aib-QGT-(αMe-2FPhe)-T-(αMe-Ser)-D-(αMe-Bip(OMe))-(αMe-APPA) |
| 168 | VH-(αMe-Pro)-QGT-(αMe-2FPhe)-T-(αMe-Ser)-D-(αMe-Bip(OMe))-(αMe-APPA) |
| 169 | VHSEGT-(αMe-2FPhe)-T-(αMe-Ser)-D-(αMe-Bip(OMe))-(αMe-APPA) |
| 170 | VHAEGT-(αMe-2FPhe)-T-(αMe-Ser)-D-(αMe-Bip(OMe))-(αMe-APPA) |
| 171 | VH-Aib-EGT-(αMe-2FPhe)-T-(αMe-Ser)-D-(αMe-Bip(OMe))-(αMe-APPA) |
| 172 | VH-(αMe-Pro)-EGT-(αMe-2FPhe)-T-(αMe-Ser)-D-(αMe-Bip(OMe))-(αMe-APPA) |
| 173 | VHSQGT-(αMe-2,6-diF-Phe)-T-(αMe-Ser)-D-(αMe-Bip(OMe))-(αMe-APPA) |
| 174 | VHAQGT-(αMe-2,6-diFPhe)-T-(αMe-Ser)-D-(αMe-Bip(OMe))-(αMe-APPA) |
| 175 | VH-Aib-QGT-(αMe-2,6-diFPhe)-T-(αMe-Ser)-D-(αMe-Bip(OMe))-(αMe-APPA) |
| 176 | VH-(αMe-Pro)-QGT-(αMe-2,6-diFPhe)-T-(αMe-Ser)-D-(αMe-Bip(OMe))-(αMe-APPA) |
| 177 | VHSEGT-(αMe-2,6-diFPhe)-T-(αMe-Ser)-D-(αMe-Bip(OMe))-(αMe-APPA) |
| 178 | VHAEGT-(αMe-2,6-diFPhe)-T-(αMe-Ser)-D-(αMe-Bip(OMe))-(αMe-APPA) |
| 179 | VH-Aib-EGT-(αMe-2,6-diFPhe)-T-(αMe-Ser)-D-(αMe-Bip(OMe))-(αMe-APPA) |
| 180 | VH-(αMe-Pro)-EGT-(αMe-2,6-diFPhe)-T-(αMe-Ser)-D-(αMe-Bip(OMe))-(αMe-APPA) |

TABLE 2 (viii)
List of peptidomimetics prepared

| Seq. | |
|---|---|
| ID. No. | Sequence of Peptidomimetics |
| 181 | VHSQGTFTS-(αMe-Asp)-(αMe-Bip(OMe))-(αMe-APPA) |
| 182 | VHAQGTFTS-(αMe-Asp)-(αMe-Bip(OMe))-(αMe-APPA) |
| 183 | VH-Aib-QGTFTS-(αMe-Asp)-(αMe-Bip(OMe))-(αMe-APPA) |
| 184 | VH-(αMe-Pro)-QGTFTS-(αMe-Asp)-(αMe-Bip(OMe))-(αMe-APPA) |
| 185 | VHSEGTFTS-(αMe-Asp)-(αMe-Bip(OMe))-(αMe-APPA) |
| 186 | VHAEGTFTS-(αMe-Asp)-(αMe-Bip(OMe))-(αMe-APPA) |
| 187 | VH-Aib-EGTFTS-(αMe-Asp)-(αMe-Bip(OMe))-(αMe-APPA) |
| 188 | VH-(αMe-Pro)-EGTFTS-(αMe-Asp)-(αMe-Bip(OMe))-(αMe-APPA) |
| 189 | VHSQGT-(αMe-2FPhe)-TS-(αMe-Asp)-(αMe-Bip(OMe))-(αMe-APPA) |
| 190 | VHAQGT-(αMe-2FPhe)-TS-(αMe-Asp)-(αMe-Bip(OMe))-(αMe-APPA) |
| 191 | VH-Aib-QGT-(αMe-2FPhe)-TS-(αMe-Asp)-(αMe-Bip(OMe))-(αMe-APPA) |
| 192 | VH-(αMe-Pro)-QGT-(αMe-2FPhe)-TS-(αMe-Asp)-(αMe-Bip(OMe))-(αMe-APPA) |
| 193 | VHSEGT-(αMe-2FPhe)-TS-(αMe-Asp)-(αMe-Bip(OMe))-(αMe-APPA) |
| 194 | VHAEGT-(αMe-2FPhe)-TS-(αMe-Asp)-(αMe-Bip(OMe))-(αMe-APPA) |
| 195 | VH-Aib-EGT-(αMe-2FPhe)-TS-(αMe-Asp)-(αMe-Bip(OMe))-(αMe-APPA) |
| 196 | VH-(αMe-Pro)-EGT-(αMe-2FPhe)-TS-(αMe-Asp)-(αMe-Bip(OMe))-(αMe-APPA) |
| 197 | VHSQGT-(αMe-2,6-diF-Phe)-TS-(αMe-Asp)-(αMe-Bip(OMe))-(αMe-APPA) |
| 198 | VHAQGT-(αMe-2,6-diFPhe)-TS-(αMe-Asp)-(αMe-Bip(OMe))-(αMe-APPA) |
| 199 | VH-Aib-QGT-(αMe-2,6-diFPhe)-TS-(αMe-Asp)-(αMe-Bip(OMe))-(αMe-APPA) |
| 200 | VH-(αMe-Pro)-QGT-(αMe-2,6-diFPhe)-TS-(αMe-Asp)-(αMe-Bip(OMe))-(αMe-APPA) |
| 201 | VHSEGT-(αMe-2,6-diFPhe)-TS-(αMe-Asp)-(αMe-Bip(OMe))-(αMe-APPA) |
| 202 | VHAEGT-(αMe-2,6-diFPhe)-TS-(αMe-Asp)-(αMe-Bip(OMe))-(αMe-APPA) |
| 203 | VH-Aib-EGT-(αMe-2,6-diFPhe)-TS-(αMe-Asp)-(αMe-Bip(OMe))-(αMe-APPA) |
| 204 | VH-(αMe-Pro)-EGT-(αMe-2,6-diFPhe)-TS-(αMe-Asp)-(αMe-Bip(OMe))-(αMe-APPA) |

TABLE 2 (ix)
List of peptidomimetics prepared

| Seq. | |
|---|---|
| ID. No. | Sequence of Peptidomimetics |
| 205 | VVHSQGTFTSD-(αMe-Bip(OMe))-(APPA) |
| 206 | VVHAQGTFTSD-(αMe-Bip(OMe))-(APPA) |
| 207 | VVH-Aib-QGTFTSD-(αMe-Bip(OMe))-(APPA) |
| 208 | VVH-(αMe-Pro)-QGTFTSD-(αMe-Bip(OMe))-(APPA) |
| 209 | VVHSEGTFTSD-(αMe-Bip(OMe))-(APPA) |
| 210 | VVHAEGTFTSD-(αMe-Bip(OMe))-(APPA) |
| 211 | VVH-Aib-EGTFTSD-(αMe-Bip(OMe))-(APPA) |
| 212 | VVH-(αMe-Pro)-EGTFTSD-(αMe-Bip(OMe))-(APPA) |
| 213 | VVHSQGT-(αMe-2FPhe)-TSD-(αMe-Bip(OMe))-(APPA) |
| 214 | VVHAQGT-(αMe-2FPhe)-TSD-(αMe-Bip(OMe))-(APPA) |
| 215 | VVH-Aib-QGT-(αMe-2FPhe)-TSD-(αMe-Bip(OMe))-(APPA) |
| 216 | VVH-(αMe-Pro)-QGT-(αMe-2FPhe)-TSD-(αMe-Bip(OMe))-(APPA) |
| 217 | VVHSEGT-(αMe-2FPhe)-TSD-(αMe-Bip(OMe))-(APPA) |
| 218 | VVHAEGT-(αMe-2FPhe)-TSD-(αMe-Bip(OMe))-(APPA) |
| 219 | VVH-Aib-EGT-(αMe-2FPhe)-TSD-(αMe-Bip(OMe))-(APPA) |
| 220 | VVH-(αMe-Pro)-EGT-(αMe-2FPhe)-TSD-(αMe-Bip(OMe))-(APPA) |
| 221 | VVHSQGT-(αMe-2,6-diF-Phe)-TSD-(αMe-Bip(OMe))-(APPA) |
| 222 | VVHAQGT-(αMe-2,6-diFPhe)-TSD-(αMe-Bip(OMe))-(APPA) |
| 223 | VVH-Aib-QGT-(αMe-2,6-diFPhe)-TSD-(αMe-Bip(OMe))-(APPA) |
| 224 | VVH-(αMe-Pro)-QGT-(αMe-2,6-diFPhe)-TSD-(αMe-Bip(OMe))-(APPA) |
| 225 | VVHSEGT-(αMe-2,6-diFPhe)-TSD-(αMe-Bip(OMe))-(APPA) |
| 226 | VVHAEGT-(αMe-2,6-diFPhe)-TSD-(αMe-Bip(OMe))-(APPA) |
| 227 | VVH-Aib-EGT-(αMe-2,6-diFPhe)-TSD-(αMe-Bip(OMe))-(APPA) |
| 228 | VVH-(αMe-Pro)-EGT-(αMe-2,6-diFPhe)-TSD-(αMe-Bip(OMe))-(APPA) |

In accordance with the present invention, the synthetic isolated peptidomimetics described herein primarily act as a glucagon receptor antagonist. Furthermore, it was found that these peptidomimetics also act as GLP-1 receptor agonists. These synthetic peptidomimetics exhibit desirable in vitro glucagon receptor antagonist properties as well as GLP-1 receptor agonist activity in CHO cells transfected with human glucagon or GLP-1 receptor (H Glucagon R or HGLP-1R), in the range of 1-100 nM concentration. The H GLP-1 R agonistic activity is assessed by estimation of amount of cAMP released, while glucagon antagonistic activity was assessed by measuring the amount of cAMP production inhibited by the test peptidomimetics, in the presence of glucagon peptide. Novel peptidomimetics exhibit desirable in vitro glucagon receptor antagonist activity in CHO cells transfected with human glucagon receptor, in the range of 1-100 nM concentrations. Some of the test peptidomimetics prepared showed glucose dependent insulin release and reduces fasting hyperglycemia, without causing hypoglycemia, when tested in vivo, in different diabetic animal models, such as hyperglycemic C57 mice and db/db mice, thus making them ideal therapeutic candidates for the treatment and prevention of type 2 diabetes. These new classes of peptidomimetics can be administered by oral or parenteral routes of administration.

### In Vitro and In Vivo Studies of Novel Peptidomimetics:

The peptidomimetics prepared as described above were tested for:
a) In vitro glucose-dependent insulin secretion (RIN5F cell assay screening protocol);
b) In vitro Human GLP-1 R agonist activity (Cyclic AMP determination);
c) In vitro human glucagon antagonist activity (Cyclic AMP determination);
d) Stability of peptidomimetics against DPP IV enzyme, human plasma, simulated gastric fluid, intestinal fluid and liver microsomes; and
e) Demonstration of in vivo efficacy of test compounds (peptidomimetics) in C57BL/6J mice (in vivo), using various in vitro and in vivo assays, as described below.

### In Vitro Studies:

a) In vitro glucose-dependent insulin secretion (RIN5F cell assay screening protocol):

RIN5F (Rat Insulinoma) cells were cultured in RPMI 1640 medium supplemented with sodium pyruvate (1 mM) HEPES and Glucose (4.5 g/L) in a humidified incubator (5% CO₂), at 37° C. After trypsinization, RIN5F cells were seeded at a concentration of 0.2×10⁶ cells per well, in 12 well plates. The cells were grown overnight to 80% confluence and insulin secretion experiments were performed as follows (Montrose-Rafizadeh C., et al., Mol. Cell. Endo. 1997, 130, 109.; Wang, X., et al., Endocrinology 2001, 5, 1820).

The cells were washed once with PBS solution followed by 40 min. incubation in fresh Krebs-Ringer Balanced Buffer containing NaCl (115 mmol/L), KCl (4.7 mmol/L), CaCl₂ (1.28 mmol/L), MgSO₄.7H₂O (1.2 mmol/L), KH₂PO₄ (1.2 mmol/L), NaHCO₃ (10 mmol/L) and HEPES (25 mmol/L), containing Glucose (1.1 mM) and B.S.A (0.5%), pH 7.4. The buffer was replaced after 40 min. and the cells were incubated (37° C.) with the test peptidomimetics, at different concentration, for 30 min., both in the presence (16.7 mM) and absence (0 mM) of glucose load. The supernatant was collected and the insulin amount was measured by ultra sensitive Rat insulin ELISA kit (Crystal Chem, IL). The protein was estimated in the supernatant using Bicinchoninic Acid kit, according to the manufacturer's protocol (Sigma Aldrich, MO). The total insulin content obtained in Pico-gram (pg) was divided with the total protein (μg) in order to normalize for differences in cell density between wells. In vitro glucose dependent insulin secretion activities of representative peptidomimetics are listed in Table 3.

TABLE 3
In vitro glucose dependent insulin secretion activity of representative
peptidomimetics

| | Conc. of test | |
|---|---|---|
| Seq. ID. No. | compd. (nM) | Insulin secretion (pg/μg/hr)* |
| Control 1 (0 mM | | 5.8 ± 0.59 |
| glucose) | | |
| Control 2 (16.7 mM | | 10.1 ± 0.47 |
| glucose) | | |
| Exendin-4 | 0.1/1/10 | 16.3 ± 0.11/21.9 ± 0.10/36.1 ± 0.09 |
| 5 | 0.1/1/10 | 12.1 ± 0.51/14.1 ± 0.19/28.1 ± 0.29 |
| 8 | 0.1/1/10 | 12.6 ± 0.25/15.3 ± 0.31/29.7 ± 0.48 |
| 10 | 0.1/1/10 | 12.0 ± 0.14/14.3 ± 0.12/27.8 ± 0.32 |
| 15 | 0.1/1/10 | 15.3 ± 0.19/21.2 ± 0.11/35.5 ± 0.19 |
| 20 | 0.1/1/10 | 11.1 ± 0.12/13.1 ± 0.13/19.0 ± 0.16 |
| 25 | 0.1/1/10 | 12.6 ± 0.20/15.9 ± 0.31/28.4 ± 0.11 |
| 28 | 0.1/1/10 | 11.8 ± 0.50/14.9 ± 0.11/27.9 ± 0.31 |
| 31 | 0.1/1/10 | 12.7 ± 0.21/15.8 ± 0.33/29.3 ± 0.19 |
| 38 | 0.1/1/10 | 13.1 ± 0.11/14.9 ± 0.17/28.8 ± 0.44 |
| 50 | 0.1/1/10 | 12.9 ± 0.14/15.8 ± 0.13/29.9 ± 0.15 |
| 58 | 0.1/1/10 | 16.1 ± 0.22/22.1 ± 0.26/36.0 ± 0.36 |
| 60 | 0.1/1/10 | 15.6 ± 0.33/21.8 ± 0.16/35.6 ± 0.26 |
| 62 | 0.1/1/10 | 16.6 ± 0.41/22.9 ± 0.32/36.7 ± 0.11 |
| 66 | 0.1/1/10 | 16.5 ± 0.12/22.7 ± 0.17/36.5 ± 0.05 |
| 69 | 0.1/1/10 | 16.2 ± 0.13/22.4 ± 0.19/36.2 ± 0.09 |
| 71 | 0.1/1/10 | 15.6 ± 0.30/21.8 ± 0.11/35.6 ± 0.20 |
| 75 | 0.1/1/10 | 16.4 ± 0.12/22.7 ± 0.17/36.6 ± 0.05 |
| 78 | 0.1/1/10 | 16.3 ± 0.15/22.4 ± 0.21/36.2 ± 0.11 |
| 81 | 0.1/1/10 | 16.8 ± 0.20/22.7 ± 0.29/36.6 ± 0.31 |
| 85 | 0.1/1/10 | 12.1 ± 0.52/14.1 ± 0.16/28.0 ± 0.36 |
| 92 | 0.1/1/10 | 16.9 ± 0.22/22.8 ± 0.31/36.7 ± 0.34 |
| 103 | 0.1/1/10 | 12.3 ± 0.33/14.1 ± 0.36/28.0 ± 0.16 |
| 108 | 0.1/1/10 | 11.9 ± 0.17/14.2 ± 0.13/27.6 ± 0.16 |
| 112 | 0.1/1/10 | 12.3 ± 0.33/14.8 ± 0.16/28.1 ± 0.22 |
| 119 | 0.1/1/10 | 12.4 ± 0.22/15.2 ± 0.32/29.6 ± 0.50 |
| 126 | 0.1/1/10 | 16.6 ± 0.41/22.9 ± 0.32/36.6 ± 0.19 |
| 130 | 0.1/1/10 | 15.6 ± 0.33/21.8 ± 0.16/35.6 ± 0.26 |
| 140 | 0.1/1/10 | 16.6 ± 0.41/22.9 ± 0.32/36.7 ± 0.11 |
| 160 | 0.1/1/10 | 16.8 ± 0.20/22.7 ± 0.29/36.6 ± 0.31 |
| 165 | 0.1/1/10 | 12.1 ± 0.42/14.6 ± 0.41/27.8 ± 0.46 |
| 168 | 0.1/1/10 | 11.9 ± 0.17/14.2 ± 0.13/27.6 ± 0.16 |
| 170 | 0.1/1/10 | 12.3 ± 0.33/14.8 ± 0.16/28.1 ± 0.22 |
| 175 | 0.1/1/10 | 12.4 ± 0.22/15.2 ± 0.32/29.6 ± 0.50 |
| 178 | 0.1/1/10 | 17.1 ± 0.11/23.1 ± 0.16/37.0 ± 0.29 |
| 182 | 0.1/1/10 | 13.1 ± 0.11/14.9 ± 0.17/28.8 ± 0.44 |
| 188 | 0.1/1/10 | 12.9 ± 0.14/15.8 ± 0.13/29.9 ± 0.15 |
| 191 | 0.1/1/10 | 16.1 ± 0.22/22.1 ± 0.26/36.0 ± 0.36 |
| 201 | 0.1/1/10 | 15.4 ± 0.14/21.2 ± 0.18/35.6 ± 0.17 |
| 205 | 0.1/1/10 | 12.6 ± 0.25/15.3 ± 0.31/29.7 ± 0.48 |
| 210 | 0.1/1/10 | 16.3 ± 0.15/22.4 ± 0.21/36.2 ± 0.11 |

*In vitro glucose dependent (16.7-mM glucose load) insulin secretion with various concentrations of peptidomimetics were measured using Rat Insulinoma (RIN5F) cells. The total insulin content (pg) was divided with total protein (μg) to normalize difference in cell density between wells. n = 3, values represent mean ± .S.D. Basal insulin secretion was observed for all the test compounds at 0-mM glucose concentration.

b) In vitro Human GLP-1 R Agonist Activity (Cyclic AMP Determination):

The novel peptidomimetics were screened for Human GLP-1 receptor (HGLP-1 R) agonist activity (in vitro), using the cAMP cell-based assay, in stably transfected CHO/human GLP1R cells. The CHO-K1 cells (CRL 9618) were obtained from American Type Culture Collection (Rockville, Md.). CHO cells were grown in Ham's F12 medium containing L-Glutamine (2 mM), HEPES (25 mM), NaHCO₃ (1.1 g/L) and supplemented with NewBorn Calf Serum (NBCS; 10%), Penicillin (50 U/ml (v/v)) and Streptomycin (50 ug/ml (v/v)). Cells were split every 3 days 1:8.

### Production of Stable Cho Cell Lines Expressing the Human GLP-1 Receptor.

The cDNA encoding the human GLP-1 receptor was isolated by RT-PCR according to standard protocol. The full-length cDNA was cloned in pcDNA3.1 (+). For the production of CHO cell lines expressing the GLP-1 receptor, CHO cells were transfected with 10 g of the expression plasmid pcDNA/hGLP-1R using CaPO₄ according to the standard protocol (Wheeler, M. B., et al., Endocrinology 1993, 133, 57.). The clones expressing the receptor were generated by G418 (800 μg/ml active, Sigma) selection. The stable clones were thereafter maintained at 500 ug/ml (G418).

The selected clone was used between passages 9-25 for cAMP assays.

Determination of cAMP Generation.

The CHO cells stably transfected with human GLP-1R were maintained in Ham's F12+10% NBCS+500 ug/ml G418 upto a confluency of 70-75%. The cells were trypsinized using 2 ml of TPVG (0.25% trypsin, 0.53 mM EDTA, 1.38-mM glucose). The trypsin was inactivated using Ham's F12 medium containing 10% NBCS and the cells were suspended in 2 ml of complete medium. 2×10⁵ cells/well were then seeded in 12 well plate and the plates were incubated in humidified atmosphere at 37° C. for 16-18 h (Fehmann, H. C., et al., Peptides 1994, 15, 453). The next day the assay was proceeded, when the cells showed 90-95% confluency. The medium was aspirated off from the 12 well plate and the cells were washed once using Ham's F12 (plain). The cells were incubated at 37° C. with 500 ul of Ham's F12+1% BSA+0.125 mM RO-20 for 30 min. After the incubation, the medium was aspirated off and fresh medium (plain Ham's F12+1% BSA+0.25 mM RO-20) was added with 5 ul of test compounds (peptidomimetics) that has been dissolved in water (MilliQ). The cells were incubated with the test compounds for 30 min in humidified atmosphere and 37° C. After the incubation, the medium was removed and the cells were washed once with plain Ham's F12. Subsequently, the cells were lysed by adding 500 ul of ice cold 0.1 N HCl to each well and shaking for 30 minutes at 200 rpm. The cells were then scrapped, the lysate was collected in micro centrifuge tubes and centrifuged at 12000 rpm for 10 min to remove the debris. 300 ul of supernatant from each micro-centrifuge tube was then removed into a glass tube and dried under N₂ for 30 min, for cAMP estimation. The total cAMP was estimated from the sample according to the manufacturer's protocol using Cyclic AMP immunoassay kit (R&D systems, Minneapolis. MN). The remaining supernatant was used to determine the protein concentration using micro BCA (Sigma). Data was calculated as percent of control (Vehicle: water) and expressed as Mean±SD. The in-vitro human GLP-1 receptor agonistic activities of representative peptidomimetics are listed in Table 4.

TABLE 4
In vitro Human GLP-1 R activity (cAMP release) of test compounds
(peptidomimetics), shown as % activity with respect to control.

| | Concentration of test compounds | | | | |
|---|---|---|---|---|---|
| Seq. ID. No. | 1 nM | 10 nM | 100 nM | 1 μM | 10 μM |
| Exendin-4 | 82 ± 0.10 | 94 ± 0.02 | 101 ± 0.09 | 100 ± 0.08 | 103 ± 0.07 |
| 7 | 55 ± 0.16 | 88 ± 0.13 | 92 ± 0.11 | 99 ± 0.07 | 99 ± 0.09 |
| 9 | 99 ± 0.12 | 99 ± 0.15 | 99 ± 0.18 | 99 ± 0.03 | 99 ± 0.09 |
| 11 | 39 ± 0.10 | 81 ± 0.08 | 89 ± 0.09 | 96 ± 0.11 | 99 ± 0.16 |
| 12 | 51 ± 0.03 | 86 ± 0.40 | 91 ± 0.21 | 99 ± 0.32 | 99 ± 0.21 |
| 14 | 55 ± 0.16 | 89 ± 0.05 | 93 ± 0.09 | 99 ± 0.02 | 99 ± 0.04 |
| 16 | 99 ± 0.03 | 99 ± 0.01 | 99 ± 0.06 | 99 ± 0.09 | 99 ± 0.11 |
| 18 | 99 ± 0.11 | 99 ± 0.13 | 99 ± 0.16 | 99 ± 0.06 | 99 ± 0.10 |
| 26 | 99 ± 0.12 | 99 ± 0.08 | 99 ± 0.11 | 99 ± 0.12 | 99 ± 0.16 |
| 34 | 99 ± 0.02 | 99 ± 0.26 | 99 ± 0.31 | 99 ± 0.60 | 99 ± 0.08 |
| 49 | 38 ± 0.12 | 78 ± 0.15 | 86 ± 0.18 | 95 ± 0.03 | 98 ± 0.09 |
| 59 | 39 ± 0.11 | 80 ± 0.09 | 88 ± 0.06 | 96 ± 0.14 | 99 ± 0.19 |
| 64 | 45 ± 0.022 | 84 ± 0.46 | 90 ± 0.41 | 99 ± 0.66 | 99 ± 0.03 |
| 68 | 40 ± 0.09 | 81 ± 0.07 | 87 ± 0.04 | 95 ± 0.01 | 98 ± 0.08 |
| 73 | 62 ± 0.11 | 94 ± 0.09 | 99 ± 0.06 | 99 ± 0.14 | 99 ± 0.19 |
| 76 | 66 ± 0.022 | 97 ± 0.46 | 99 ± 0.41 | 99 ± 0.66 | 99 ± 0.03 |
| 79 | 69 ± 0.09 | 98 ± 0.07 | 99 ± 0.04 | 99 ± 0.01 | 99 ± 0.08 |
| 84 | 40 ± 0.09 | 81 ± 0.07 | 87 ± 0.04 | 95 ± 0.01 | 98 ± 0.08 |
| 88 | 38 ± 0.11 | 77 ± 0.16 | 85 ± 0.11 | 94 ± 0.08 | 97 ± 0.05 |
| 89 | 93 ± 0.11 | 99 ± 0.09 | 99 ± 0.06 | 99 ± 0.14 | 99 ± 0.19 |
| 91 | 93 ± 0.10 | 99 ± 0.46 | 99 ± 0.41 | 99 ± 0.66 | 99 ± 0.03 |
| 99 | 99 ± 0.06 | 99 ± 0.06 | 99 ± 0.08 | 99 ± 0.10 | 99 ± 0.12 |
| 105 | 99 ± 0.11 | 99 ± 0.13 | 99 ± 0.16 | 99 ± 0.06 | 99 ± 0.10 |
| 110 | 99 ± 0.12 | 99 ± 0.08 | 99 ± 0.11 | 99 ± 0.12 | 99 ± 0.16 |
| 118 | 99 ± 0.02 | 99 ± 0.26 | 99 ± 0.31 | 99 ± 0.60 | 99 ± 0.08 |
| 122 | 96 ± 0.09 | 99 ± 0.07 | 99 ± 0.04 | 99 ± 0.01 | 99 ± 0.08 |
| 124 | 39 ± 0.10 | 81 ± 0.08 | 89 ± 0.09 | 96 ± 0.11 | 99 ± 0.16 |
| 129 | 51 ± 0.03 | 86 ± 0.40 | 91 ± 0.21 | 99 ± 0.32 | 99 ± 0.21 |
| 139 | 55 ± 0.16 | 89 ± 0.05 | 93 ± 0.09 | 99 ± 0.02 | 99 ± 0.04 |
| 142 | 60 ± 0.12 | 92 ± 0.15 | 98 ± 0.18 | 99 ± 0.03 | 99 ± 0.09 |
| 145 | 78 ± 0.12 | 99 ± 0.15 | 99 ± 0.18 | 99 ± 0.03 | 99 ± 0.09 |
| 149 | 38 ± 0.11 | 77 ± 0.16 | 85 ± 0.11 | 94 ± 0.08 | 97 ± 0.05 |
| 162 | 41 ± 0.09 | 82 ± 0.07 | 87 ± 0.04 | 95 ± 0.01 | 98 ± 0.08 |
| 164 | 38 ± 0.11 | 77 ± 0.16 | 85 ± 0.11 | 94 ± 0.08 | 97 ± 0.05 |
| 166 | 39 ± 0.10 | 81 ± 0.08 | 89 ± 0.09 | 96 ± 0.11 | 99 ± 0.16 |
| 172 | 60 ± 0.12 | 92 ± 0.15 | 98 ± 0.18 | 99 ± 0.03 | 99 ± 0.09 |
| 177 | 86 ± 0.11 | 99 ± 0.09 | 99 ± 0.06 | 99 ± 0.14 | 99 ± 0.19 |
| 187 | 96 ± 0.02 | 99 ± 0.46 | 99 ± 0.41 | 99 ± 0.66 | 99 ± 0.03 |
| 197 | 96 ± 0.09 | 99 ± 0.07 | 99 ± 0.04 | 99 ± 0.01 | 99 ± 0.08 |
| 207 | 39 ± 0.12 | 80 ± 0.15 | 86 ± 0.18 | 95 ± 0.03 | 98 ± 0.09 |
| 218 | 40 ± 0.11 | 81 ± 0.09 | 89 ± 0.06 | 96 ± 0.14 | 99 ± 0.19 |
| 222 | 38 ± 0.12 | 78 ± 0.15 | 86 ± 0.18 | 95 ± 0.03 | 98 ± 0.09 |
| 228 | 39 ± 0.11 | 80 ± 0.09 | 88 ± 0.06 | 96 ± 0.14 | 99 ± 0.19 |

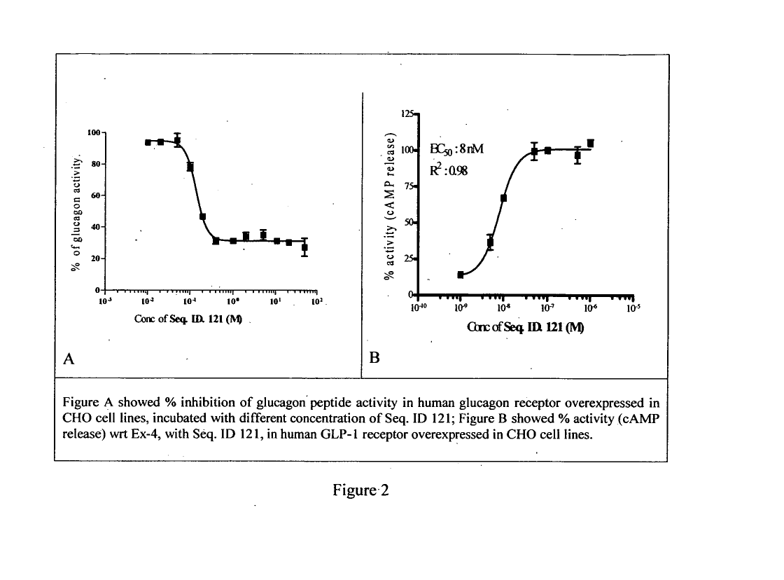

Based upon the in-vitro human GLP-1 receptor agonistic activity, EC₅₀ values were determined for novel peptidomimetics and the comparative dose-response curves (DRC) for Seq. ID. No. 121: VH-Aib-EGT-(αMe-2FPhe)-T-(αMe-Ser)-(αMe-Asp)-Bip(OMe)-(αMe-APPA); (EC₅₀=8 nM) are shown in FIG. 2 as representative example.

c) In Vitro Human Glucagon Antagonist Activity (Measurement of Inhibition of Amount of Cyclic AMP Production, with Test Peptidomimetics):

The novel peptidomimetics were screened for human glucagon receptor (H-glucagon-R) antagonistic activity (in vitro), using the cAMP cell-based assay, in stably transfected CHO/human glucagon R cells. The CHO-K1 cells (CRL 9618) were obtained from American Type Culture Collection (Rockville, Md.). CHO cells were grown in Ham's F12 medium containing L-Glutamine (2 mM), HEPES (25 mM), NaHCO₃ (1.1 g/L) and supplemented with newborn Calf Serum (NBCS; 10%), Penicillin (50 U/ml (v/v)) and Streptomycin (50 ug/ml (v/v)). Cells were split every 3 days 1:8.

### Production of Stable CHO Cell Lines Expressing the Human Glucagon Receptor.

The cDNA encoding the human glucagon receptor was isolated by RT-PCR according to standard protocol. The full-length cDNA was cloned in pcDNA3.1 (Invitrogen). For the production of CHO cell lines expressing the glucagon receptor, CHO cells were transfected with 10 μg of the expression plasmid pcDNA/H-glucagon-R using CaPO₄ according to the standard protocol. The clones expressing the receptor were generated by G418 (800 μg/ml active, Sigma) selection. The stable clones were thereafter maintained at 500 ug/ml (G418). The selected clone was used between passages 9-25 for cAMP assays.

Determination of Glucagon Antagonistic Activity by Measuring Amount of cAMP Production Inhibited after Addition of Test Peptidomimetics Along with Glucagon Peptide.

The CHO cells stably transfected with human glucagon R were maintained in Ham's F12+10% NBCS+500 ug/ml G418 upto a confluency of 70-75%. The cells were trypsinized using 2 ml of TPVG (0.25% trypsin, 0.53 mM EDTA, 1.38-mM glucose). The trypsin was inactivated using Ham's F12 medium containing 10% NBCS and the cells were suspended in 2 ml of complete medium. 2×10⁵ cells/well were then seeded in 12 well plate and the plates were incubated in humidified atmosphere at 37° C. for 16-18 h. The next day, the assay was proceeded when the cells showed 90-95% confluency. The medium was aspirated off from the 12 well plate and the cells were washed once using Ham's F12 (plain). The cells were incubated at 37° C. with 500 ul of Ham's F12+1% BSA+0.125 mM RO-20 for 30 min. After the incubation, the medium was aspirated off and a fresh medium (plain Ham's F12+1% BSA+0.25 mM RO-20) was added with 5 ul of test compounds (peptidomimetics) that has been dissolved in water (MilliQ), followed by the addition of glucagon peptide (as agonist). The cells were incubated with the peptidomimetics and glucagon peptide for 30 min in humidified atmosphere and 37° C. After the incubation, the medium was removed and the cells were washed once with plain Ham's F12. Subsequently, the cells were lysed by adding 500 ul of ice cold 0.1 N HCl to each well and shaking for 30 minutes at 200 rpm. The cells were then scrapped, the lysate was collected in micro centrifuge tubes and centrifuged at 12000 rpm for 10 min to remove the debris. 300 ul of supernatant from each micro-centrifuge tube was then removed into a glass tube and dried under N₂ for 30 min, for cAMP estimation. The total cAMP was estimated from the sample according to the manufacturer's protocol using Cyclic AMP immunoassay kit (R&D systems, Minneapolis. MN). The remaining supernatant is used to determine the protein concentration using micro BCA (Sigma). Data is calculated as percent of control (Vehicle: water) and expressed as Mean+SD. The in-vitro human glucagon receptor antagonistic activities of representative peptidomimetics are listed in Table 5.

TABLE 5
In vitro Human Glucagon receptor antagonistic activity of test compounds
(peptidomimetics) shown as inhibition of cAMP production (pmol/ml/μg prt) of
Glucagon peptide, by the test compounds, incubated at different concentration, along
with saturated concentration of glucagon peptide.

| | Concentration of test compounds | | | | |
|---|---|---|---|---|---|
| Seq. ID. No. | 1 nM | 10 nM | 100 nM | 1 μM | 10 μM |
| Glucagon | 22 ± 0.09 | 35 ± 0.10 | 37.8 ± 0.01 | 37.1 ± 0.02 | 37.4 ± 0.06 |
| 13 | 20 ± 0.06 | 18 ± 0.09 | 16 ± 0.01 | 14 ± 0.03 | 12 ± 0.01 |
| 17 | 6 ± 0.02 | 5 ± 0.03 | 2 ± 0.61 | 0 | 0 |
| 19 | 15 ± 0.11 | 12 ± 0.13 | 8 ± 0.09 | 4 ± 0.01 | 2 ± 0.08 |
| 23 | 10 ± 0.03 | 8 ± 0.03 | 6 ± 0.22 | 2 ± 0.13 | 0 |
| 30 | 5 ± 0.02 | 3 ± 0.15 | 0 | 0 | 0 |
| 33 | 0 | 0 | 0 | 0 | 0 |
| 39 | 17 ± 0.12 | 15 ± 0.13 | 12 ± 0.13 | 8 ± 0.16 | 6 ± 0.12 |
| 44 | 5 ± 0.09 | 3 ± 0.11 | 0 | 0 | 0 |
| 47 | 9 ± 0.11 | 6 ± 0.12 | 5 ± 0.14 | 3 ± 0.22 | 0 |
| 63 | 20 ± 0.06 | 18 ± 0.09 | 16 ± 0.01 | 14 ± 0.03 | 12 ± 0.01 |
| 70 | 2 ± 0.03 | 0 | 0 | 0 | 0 |
| 80 | 0 | 0 | 0 | 0 | 0 |
| 87 | 5 ± 0.04 | 3 ± 0.02 | 1 ± 0.11 | 0 | 0 |
| 90 | 22 ± 0.03 | 21 ± 0.05 | 20 ± 0.07 | 18 ± 0.04 | 18 ± 0.03 |
| 113 | 4 ± 0.01 | 2 ± 0.17 | 0 | 0 | 0 |
| 127 | 18 ± 0.07 | 16 ± 0.01 | 12 ± 0.03 | 8 ± 0.02 | 6 ± 0.08 |
| 163 | 15 ± 0.11 | 12 ± 0.13 | 8 ± 0.09 | 4 ± 0.01 | 2 ± 0.08 |
| 167 | 9 ± 0.11 | 6 ± 0.12 | 5 ± 0.14 | 3 ± 0.22 | 0 |
| 169 | 5 ± 0.01 | 2 ± 0.17 | 0 | 0 | 0 |
| 179 | 21 ± 0.11 | 19 ± 0.02 | 18 ± 0.07 | 18 ± 0.03 | 16 ± 0.02 |
| 189 | 4 ± 0.09 | 2 ± 0.12 | 0 | 0 | 0 |
| 199 | 14 ± 0.02 | 11 ± 0.03 | 7 ± 0.22 | 3 ± 0.12 | 0 |
| 200 | 10 ± 0.16 | 8 ± 0.12 | 6 ± 0.14 | 4 ± 0.15 | 1 ± 0.12 |
| 204 | 9 ± 0.14 | 6 ± 0.14 | 5 ± 0.13 | 3 ± 0.24 | 0 |
| 208 | 4 ± 0.16 | 2 ± 0.11 | 0 | 0 | 0 |
| 209 | 0 | 0 | 0 | 0 | 0 |
| 211 | 6 ± 0.05 | 5 ± 0.04 | 2 ± 0.16 | 0 | 0 |
| 216 | 0 | 0 | 0 | 0 | 0 |
| 217 | 5 ± 0.01 | 2 ± 0.17 | 0 | 0 | 0 |
| 227 | 19 ± 0.03 | 17 ± 0.08 | 15 ± 0.01 | 14 ± 0.02 | 12 ± 0.11 |

### d) Stability of Peptidomimetics Against DPP IV Enzyme, Human Plasma, Simulated Gastric Fluid, Intestinal Fluid and Liver Microsomes:

Different peptidomimetics (final concentration 2 μM) were incubated with either DPP IV (1:25 mU) or pooled human plasma (7.5 μL) or simulated gastric fluid (pH 1.5; composition HCl, NaCl and Pepsin) or simulated intestinal fluid (pH 7.5) or human liver microsomes, for 0, 2, 4, 6, 12 and 24 h (37° C.; 50 mM triethanolamine-HCl buffer; pH 7.8). Concentrations of DPP IV enzyme/human plasma/simulated gastric fluid/simulated intestinal fluid/human liver microsomes were selected in preliminary experiments to provide degradation of approximately 50% of Exendin within 2-4 h, therefore allowing time-dependent degradation to be viewed over 24 h. Reactions were terminated by the addition of TFA/H₂O (15 mL, 10% (v/v)). The reaction products were then applied to a Vydac C₁₈ analytical column (4.6×250-mm) and the major degradation fragment separated from intact peptidomimetic. The column was equilibrated with TFA/H₂0, at a flow rate of 1 mL/min. Using 0.1% (v/v) TFA in 70% acetonitrile/H₂O, the concentration of acetonitrile in the eluting solvent was raised from 0% to 28% over 10 min and from 28% to 42% over 30 min. The absorbance was monitored at 206 nm using UV detector and peaks were collected manually prior to ESI-MS analysis. Area under the curve was measured for test peptidomimetics and their metabolites and percentage degradation were calculated at each time point over a period of 24 h. The stability study results of selected peptidomimetics, against DPP IV enzyme, human plasma, simulated gastric fluid, intestinal fluid and liver microsomes (in vitro) are listed in Table 6.

TABLE 6
Stability study results of selected peptidomimetics against DPP IV
enzyme, human plasma, simulated gastric fluid, intestinal fluid and liver
microsomes (in vitro)

| Seq. | | | | | Simulated | | Simulated | | | |
|---|---|---|---|---|---|---|---|---|---|---|
| ID. | DPP IV | | Human | | gastric | | intestinal | | liver | |
| No. | enzyme^{a} | | plasma^{b} | | fluid^{c} | | fluid^{d} | | microsomes^{e} | |
| EX-4 | 90 | (5.9) | 88 | (5.5) | 100 | (0.3) | 100 | (0.1) | 100 | (0.1) |
| 28 | 70 | (9) | 71 | (8) | 14 | (8) | 40 | (6) | 78 | (1) |
| 31 | 86 | (9) | 70 | (8) | 15 | (8) | 41 | (6) | 77 | (1) |
| 38 | 00 | (>24) | 00 | (>24) | 00 | (>24) | 00 | (>24) | 31 | (5) |
| 50 | 75 | (10) | 77 | (9) | 100 | (0.5) | 100 | (0.5) | 100 | (0.5) |
| 58 | 71 | (8) | 69 | (8) | 11 | (7) | 43 | (6) | 77 | (2) |
| 62 | 00 | (>24) | 00 | (>24) | 00 | (>24) | 00 | (>24) | 35 | (5) |
| 69 | 76 | (9) | 78 | (8) | 14 | (8) | 43 | (6) | 80 | (1) |
| 75 | 74 | (9) | 75 | (8) | 12 | (8) | 46 | (6) | 83 | (1) |
| 81 | 70 | (9) | 71 | (8) | 14 | (8) | 40 | (6) | 78 | (1) |
| 92 | 00 | (>24) | 00 | (>24) | 55 | (4) | 00 | (>24) | 84 | (2) |
| 108 | 00 | (>24) | 00 | (>24) | 00 | (>24) | 00 | (>24) | 33 | (5) |
| 112 | 70 | (10) | 71 | (9) | 100 | (0.5) | 100 | (0.5) | 100 | (0.5) |
| 140 | 86 | (10) | 70 | (9) | 100 | (0.5) | 100 | (0.5) | 100 | (0.5) |
| 165 | 72 | (10) | 70 | (9) | 100 | (0.5) | 100 | (0.5) | 100 | (0.5) |
| 170 | 00 | (>24) | 00 | (>24) | 52 | (4) | 00 | (>24) | 81 | (2) |
| 178 | 00 | (>24) | 00 | (>24) | 43 | (4) | 00 | (>24) | 84 | (2) |
| 188 | 00 | (>24) | 00 | (>24) | 41 | (4) | 00 | (>24) | 80 | (2) |
| 191 | 76 | (9) | 78 | (8) | 12 | (8) | 55 | (6) | 79 | (1) |
| 201 | 75 | (9) | 77 | (8) | 14 | (8) | 45 | (6) | 81 | (1) |
| 205 | 00 | (>24) | 00 | (>24) | 43 | (4) | 00 | (>24) | 84 | (2) |

^{a}% degradation of peptidomimetics in 24 h when incubated with DPP-IV enzyme and values in bracket represent half-life (t_{1/2}), in h;
^{b}% degradation of peptidomimetics in 24 h when incubated with human plasma and values in bracket represent half-life (t_{1/2}), in h;
^{c}% degradation of peptidomimetics in 24 h when incubated with simulated gastric fluid and values in bracket represent half-life (t_{1/2}), in h;
^{d}% degradation of peptidomimetics in 24 h when incubated with simulated intestinal fluid and values in bracket represent half-life (t_{1/2}), in h;
^{e}% degradation of peptidomimetics in 24 h when incubated with liver microsomes and values in bracket represent half-life (t_{1/2}), in h.

### In Vivo Efficacy Studies:

e) Demonstration of In Vivo Efficacy (Antihyperglycaemic/Antidiabetic Activity) of Test Compounds (Peptidomimetics) in C57BL/6J or db/db Mice, Both by Parenteral (i.p) and Oral Routes of Administration.

### Animals

Acute single dose 120-min time-course experiments were carried out in male C57BL/6J or db/db mice, age 8-12 weeks, bred in-house. The animals were housed in groups of 6 animals per cage, for a week, in order to habituate them to vivarium conditions (25+4° C., 60-65% relative humidity, 12:12 h light: dark cycle, with lights on at 7.30 am). All the animal experiments were carried out according to the internationally valid guidelines following approval by the ‘Zydus Research Center animal ethical committee’.

### Procedure

The in-vivo glucose lowering properties of some of the test compounds (peptidomimetics) and Exendin-4 were evaluated in C57BL/6J (mild hyperglycemic) or db/db animal models as described below. Two days prior to the study, the animals were randomised and divided into 5 groups (n=6), based upon their fed glucose levels. On the day of experiment, food was withdrawn from all the cages, water was given ad-libitum and were kept for overnight fasting. Vehicle (normal saline)/test/standard compounds were administered intraperitoneally (i.p.) or orally, on a body weight basis. Soon after the 0 min. blood collection from each animal, the subsequent blood collections were done at 30, 60 and 120 or upto 240 min., via retro-orbital route, under light ether anesthesia (Chen, D., et al., Diabetes Obesity Metabolism, 2005, 7, 307. Kim, J. G. et al., Diabetes, 2003, 52, 751).

The blood samples were centrifuged and the separated serum was immediately subjected for the glucose estimation. The serum for insulin estimation was stored at −70° C. until used for the insulin estimation. The glucose estimation was carried out with DPEC-GOD/POD method (Ranbaxy Fine Chemicals Limited, Diagnostic division, India), using Spectramax-190, in 96-microwell plate reader (Molecular devices Corporation, Sunnyvale, Calif.). The Mean values of duplicate samples were calculated using Microsoft excel and the Graph Pad Prism software (Ver 4.0) was used to plot a 0 min base line corrected line graph, area under the curve (0-120 min AUC) and base line corrected area under the curve (0 min BCAUC). The AUC and BCAUC obtained from graphs were analyzed for one way ANOVA, followed by Dunnett's post test, using Graph Pad prism software. Furthermore, the insulin estimation was carried out using rat/mouse insulin ELISA kit (Linco research, Missouri USA). Changes in the blood glucose levels, at 0, 30, 60 and 120 min, with selected peptidomimetics are shown in Table 7 (via ip route of administration) and Table 8 (via oral route of administration), respectively.

TABLE 7
Acute single dose 120-min time-course experiments, in male C57BL/6J
mice (in vivo glucose reduction); n = 8, all values are Mean ±
SEM, via intraperitonial (i.p.) route of administration.

| Treatment group | 0 min | 30 min | 60 min | 120 min |
|---|---|---|---|---|
| C57 control | 182 ± 1.6 | 184 ± 2.5 | 186 ± 4.2 | 185 ± 2.9 |
| Exendin | 184 ± 2.5 | 114 ± 4.2 | 126 ± 3.8 | 147 ± 5.6 |
| (2 nM/kg, i.p) | | | | |
| Seq. ID. 19 | 183 ± 5.1 | 122 ± 3.1 | 130 ± 2.3 | 148 ± 2.2 |
| (50 nM/kg, i.p) | | | | |
| Seq. ID. 23 | 180 ± 5.2 | 120 ± 2.9 | 138 ± 2.4 | 149 ± 2.0 |
| (10 nM/kg, i.p) | | | | |
| Seq. ID. 30 | 181 ± 5.3 | 119 ± 3.0 | 128 ± 2.6 | 147 ± 1.9 |
| (30 nM/kg, i.p) | | | | |
| Seq. ID. 39 | 183 ± 3.6 | 117 ± 3.3 | 116 ± 3.2 | 117 ± 1.4 |
| (30 nM/kg, i.p) | | | | |
| Seq. ID. 47 | 180 ± 3.1 | 122 ± 3.9 | 129 ± 2.2 | 140 ± 2.9 |
| (20 nM/kg, i.p) | | | | |
| Seq. ID. 56 | 179 ± 5.0 | 120 ± 3.6 | 129 ± 2.0 | 148 ± 1.7 |
| (5 nM/kg, i.p) | | | | |
| Seq. ID. 63 | 182 ± 5.1 | 118 ± 2.1 | 116 ± 2.2 | 118 ± 2.4 |
| (30 nM/kg, i.p) | | | | |
| Seq. ID. 80 | 179 ± 2.9 | 121 ± 3.0 | 129 ± 3.8 | 142 ± 1.6 |
| (20 nM/kg, i.p) | | | | |
| Seq. ID. 87 | 181 ± 3.0 | 123 ± 3.8 | 128 ± 3.2 | 146 ± 1.8 |
| (5 nM/kg, i.p) | | | | |
| Seq. ID. 90 | 183 ± 4.9 | 120 ± 2.8 | 129 ± 3.0 | 139 ± 2.0 |
| (20 nM/kg, i.p) | | | | |
| Seq. ID. 113 | 182 ± 2.1 | 111 ± 2.1 | 112 ± 2.8 | 113 ± 2.2 |
| (10 nM/kg, i.p) | | | | |
| Seq. ID. 127 | 181 ± 5.1 | 120 ± 3.8 | 128 ± 2.2 | 149 ± 1.6 |
| (50 nM/kg, i.p) | | | | |
| Seq. ID. 163 | 180 ± 5.2 | 120 ± 2.9 | 138 ± 2.4 | 149 ± 2.0 |
| (5 nM/kg, i.p) | | | | |
| Seq. ID. 169 | 181 ± 5.3 | 119 ± 3.0 | 128 ± 2.6 | 147 ± 1.9 |
| (50 nM/kg, i.p) | | | | |
| Seq. ID. 179 | 182 ± 2.1 | 121 ± 2.9 | 130 ± 3.1 | 138 ± 2.4 |
| (20 nM/kg, i.p) | | | | |
| Seq. ID. 189 | 181 ± 4.1 | 117 ± 3.8 | 117 ± 3.0 | 117 ± 1.6 |
| (30 nM/kg, i.p) | | | | |
| Seq. ID. 199 | 180 ± 1.2 | 116 ± 3.2 | 116 ± 3.1 | 118 ± 1.8 |
| (30 nM/kg, i.p) | | | | |
| Seq. ID. 208 | 181 ± 5.2 | 119 ± 2.6 | 117 ± 2.1 | 119 ± 2.3 |
| (30 nM/kg, i.p) | | | | |
| Seq. ID. 209 | 182 ± 2.8 | 122 ± 3.2 | 128 ± 3.4 | 141 ± 1.7 |
| (20 nM/kg, i.p) | | | | |
| Seq. ID. 217 | 180 ± 5.2 | 119 ± 3.2 | 129 ± 2.6 | 148 ± 1.8 |
| (50 nM/kg, i.p) | | | | |
| Seq. ID. 227 | 182 ± 5.3 | 121 ± 3.3 | 127 ± 2.8 | 149 ± 2.0 |
| (5 nM/kg, i.p) | | | | |

TABLE 8
Acute single dose 120-min time-course experiments,
in male C57BL/6J mice (in vivo glucose reduction),
with selected peptidomimetics; n = 8, all values
are Mean ± SEM, via oral route of administration

| Treatment group | 0 min | 30 min | 60 min | 120 min |
|---|---|---|---|---|
| C57 control | 186 ± 7.1 | 186 ± 7.8 | 181 ± 3.0 | 173 ± 6.4 |
| Seq. ID. 49 | 184 ± 2.2 | 118 ± 3.3 | 112 ± 4.4 | 110 ± 2.9 |
| (2 μM/kg, oral) | | | | |
| Seq. ID. 59 | 184 ± 2.5 | 115 ± 3.6 | 113 ± 4.2 | 110 ± 2.4 |
| (2 μM/kg, oral) | | | | |
| Seq. ID. 64 | 183 ± 2.4 | 116 ± 3.1 | 112 ± 4.8 | 110 ± 2.5 |
| (2 μM/kg, oral) | | | | |
| Seq. ID. 68 | 184 ± 2.2 | 118 ± 3.3 | 112 ± 4.4 | 110 ± 2.9 |
| (2 μM/kg, oral) | | | | |
| Seq. ID. 76 | 183 ± 2.4 | 116 ± 3.1 | 112 ± 4.8 | 110 ± 2.5 |
| (2 μM/kg, oral) | | | | |
| Seq. ID. 84 | 183 ± 2.3 | 119 ± 3.0 | 112 ± 4.3 | 109 ± 2.6 |
| (2 μM/kg, oral) | | | | |
| Seq. ID. 89 | 183 ± 2.1 | 118 ± 3.4 | 113 ± 4.6 | 111 ± 2.8 |
| (2 μM/kg, oral) | | | | |
| Seq. ID. 99 | 184 ± 2.5 | 118 ± 3.6 | 113 ± 4.2 | 110 ± 2.4 |
| (2 μM/kg, oral) | | | | |
| Seq. ID. 105 | 185 ± 2.1 | 115 ± 2.8 | 112 ± 4.9 | 111 ± 2.2 |
| (2 μM/kg, oral) | | | | |
| Seq. ID. 118 | 184 ± 2.5 | 115 ± 1.6 | 113 ± 3.2 | 110 ± 2.4 |
| (2 μM/kg, oral) | | | | |
| Seq. ID. 129 | 183 ± 2.4 | 116 ± 3.1 | 112 ± 4.8 | 110 ± 2.5 |
| (2 μM/kg, oral) | | | | |
| Seq. ID. 139 | 182 ± 2.5 | 116 ± 3.1 | 113 ± 4.1 | 110 ± 2.8 |
| (1 μM/kg, oral) | | | | |
| Seq. ID. 145 | 184 ± 2.2 | 118 ± 3.3 | 112 ± 4.4 | 110 ± 2.9 |
| (2 μM/kg, oral) | | | | |
| Seq. ID. 162 | 183 ± 2.1 | 118 ± 3.4 | 113 ± 4.6 | 111 ± 2.8 |
| (2 μM/kg, oral) | | | | |
| Seq. ID. 166 | 184 ± 2.5 | 115 ± 3.6 | 113 ± 4.2 | 110 ± 2.4 |
| (0.5 μM/kg, oral) | | | | |
| Seq. ID. 177 | 183 ± 2.4 | 116 ± 3.1 | 112 ± 4.8 | 110 ± 2.5 |
| (2 μM/kg, oral) | | | | |
| Seq. ID. 187 | 183 ± 2.4 | 119 ± 3.1 | 112 ± 4.8 | 110 ± 2.5 |
| (2 μM/kg, oral) | | | | |
| Seq. ID. 197 | 185 ± 2.3 | 116 ± 3.8 | 113 ± 4.9 | 109 ± 2.2 |
| (2 μM/kg, oral) | | | | |
| Seq. ID. 207 | 181 ± 2.5 | 117 ± 3.3 | 112 ± 4.0 | 108 ± 2.9 |
| (2 μM/kg, oral) | | | | |
| Seq. ID. 218 | 183 ± 2.0 | 117 ± 1.1 | 112 ± 1.1 | 110 ± 1.8 |
| (1.5 μM/kg, oral) | | | | |

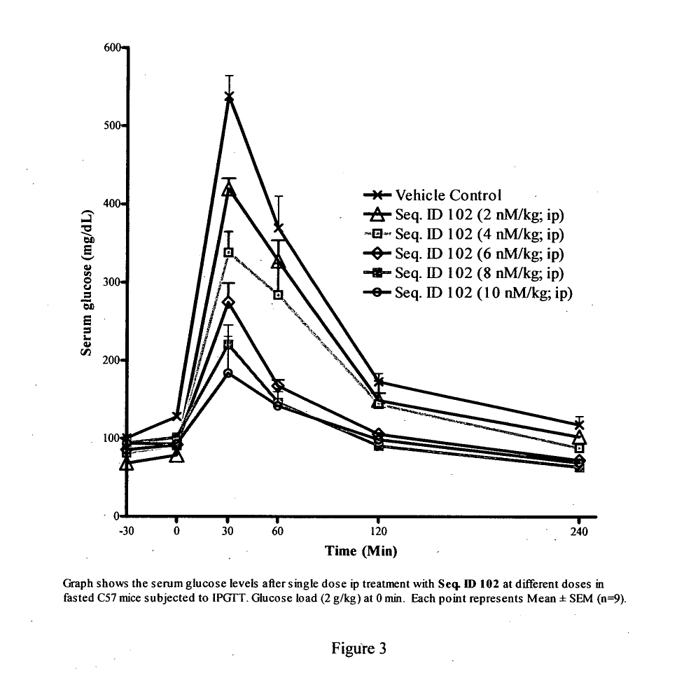

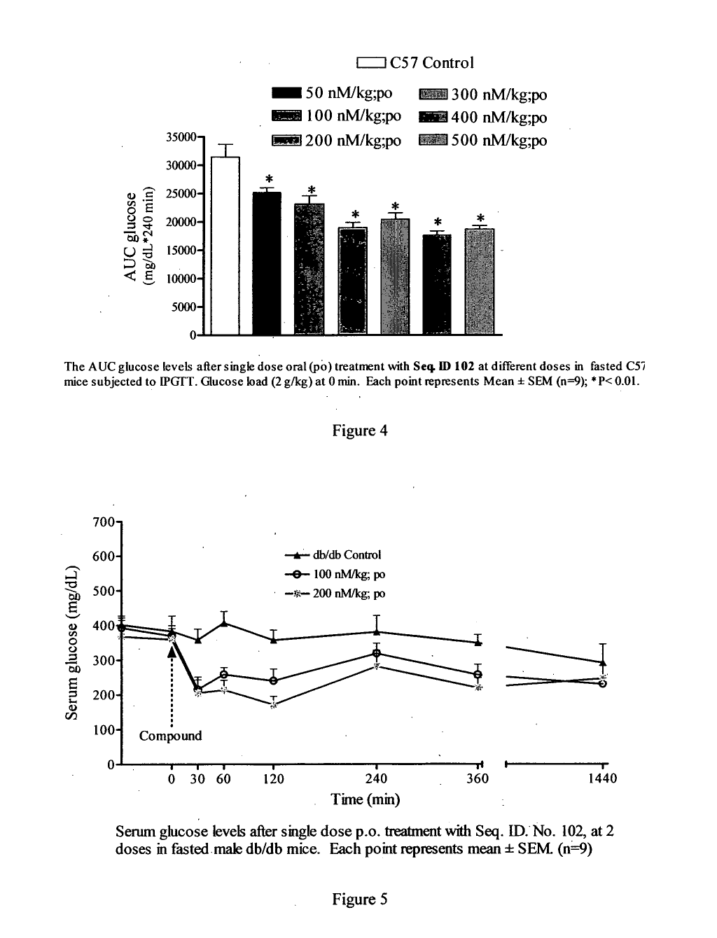

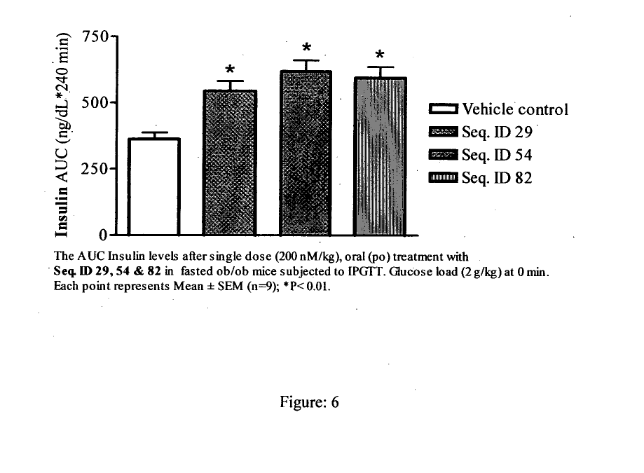

Some of the baseline corrected serum glucose levels, as representative figures are shown, after acute treatment with Seq. ID. No. 102 (VH-(αMe-Pro)-QGT-(αMe-2,6-diFPhe)-TSD-Bip(OMe)-(αMe-APPA)), at different doses (2/4/6/8/10 nM/Kg), in C57 mice, via ip route of administration (FIG. 3), via oral route of administration at 50/100/200/300/400/500 nM/Kg dose (FIG. 4) or in db/db via oral route of administration at 100 and 200 nM/kg dose (FIG. 5). FIG. 6 represents the change in serum insulin levels after acute oral administration of vehicles/test compounds (Seq. ID. No. 29 (VHSEGT-(αMe-2,6-diFPhe)-TSD-(Me-Bip(OMe))-(APPA)), Seq. ID. No. 54 (VHAEGT-(αMe-2,6-diFPhe)-T-(αMe-Ser)-D-(αMe-Bip(OMe))-(APPA)) and Seq. ID. No. 82 (VHAQGTFTSD-Bip(OMe)-(αMe-APPA)), in ob/ob mice at 200 nM/kg; po dose (in vivo).

### In Vitro and In Vivo Results of Peptidomimetics—An Overview:

As described above, all the peptidomimetics prepared in the present invention were evaluated in vitro and in vivo and the data of selected peptidomimetics were presented in the above section as examples of representative peptidomimetics. In RIN 5F (rat insulinoma) cell based assay, all the peptidomimetics showed only glucose-dependent insulin secretion, in the range of 1-10 nM concentration (Table 3), thereby these class of peptidomimetics are likely to be devoid of hyperglycemic episodes, which is commonly observed with other class of insulin secretagogues, such as sulfonylureas. In human glucagon receptor assay, in vitro antagonistic activity of peptidomemtics was estimated by measuring the inhibition of amount of cAMP production, with test peptidomemtics, when incubated along with the glucagon peptide. As shown in the Table 5, in general, all the peptidomimetics showed significant glucagon receptor antagonistic activity, in the range of 1 nM to 1000 nM. In HGLP-1R assay, the novel peptidomimetics showed concentration dependent cAMP production (in vitro GLP-1 agonist activity), in the range of 1-100 nM concentration (Table 4). This dual nature of peptidomimetics (antagonist of the glucagon receptor and agonist of the GLP-1 receptor), make them ideal candidate for the safe and effective treatment of type 2 diabetes and associated metabolic disorders.

The stability study results of selected peptidomimetics against DPP-IV enzyme, human plasma, simulated gastric and intestinal fluid and liver microsomes, indicates that most of the peptidomimetics are stable against DPP-IV enzyme, when incubated upto 24 hrs. Similarly, in human plasma, simulated gastric and intestinal fluid, most of the peptidomimetics were found to be stable, when incubated upto 24 hrs. Incubation of peptidomimetics with liver microsomes showed significant stability and only 26-35% degradation were observed in 24 hrs, indicated that some of the peptidomimetics could be delivered by oral route of administration.

In vivo antihyperglycaemic/antidiabetic activity of peptidomimetics, both by parenteral and oral route of administration were determined in C57 or db/db mice, using acute-single-dose 120/240-min time course experiment. As shown in Table 7, most of the peptidomimetics are active via i.p. route of administration, in the dose range of 5-50 nM, while orally, some of the selected peptidomimetics (Table 8) are active in the range of 0.5-2 μM/kg dose. Thus novel peptidomimetics exhibit glucagon antagonistic and GLP-1 agonistic activity and are orally bioavailable, which make them ideal candidate for the safe and effective treatment of type 2 diabetes and associated metabolic disorders.

The novel compounds of the present invention (I) may be formulated into suitable pharmaceutically acceptable compositions by combining with suitable excipients by techniques and processes and concentrations as are well known.

The compounds of Formula (I) or pharmaceutical compositions containing them are suitable for humans and other warm blooded animals, and may be administered either by oral, topical or parenteral administration or other suitable routes based on the requirement of the patients for the treatment of various disease conditions associated with dyslipidemia, obesity etc.

The pharmaceutical composition may be provided by employing conventional techniques. In particular, the composition is in unit dosage form containing an effective amount of the active component, that is, the compounds of Formula (I) according to this invention.

The quantity of active component, that is, the compounds of Formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

The synthetic peptidomimetics described in the present invention exhibit desirable in vitro glucagon antagonistic and GLP-1 agonist activity in CHO cells transfected with human glucagon or HGLP-1R, in nM concentration, and in vivo, some of the peptidomimetics showed glucose dependent insulin release and reduces fasting hyperglycemia, without causing hypoglycemia, when tested in different diabetic animal models, such as hyperglycemic C57 mice and db/db mice.

Novel peptidomimetics of the present invention showed increased stability against various proteolytic enzymes and due to increased stability and short chain length, such peptidomimetics can also be delivered by oral route of administration, along with other invasive and non-invasive routes of administration.

Accordingly, the peptidomimetics of the present invention can be administered to mammals, preferably humans, for the treatment of a variety of conditions and disorders, including, but not limited to, treating or delaying the progression or onset of diabetes (preferably type II, impaired glucose tolerance, insulin resistance and diabetic complications, such as nephropathy, retinopathy, neuropathy and cataracts), hyperglycemia, hyperinsulinemia, hypercholesterolemia, elevated blood levels of free fatty acids or glycerol, hyperlipidemia, hypertriglyceridemia, wound healing, tissue ischemia, atherosclerosis, hypertension, intestinal diseases (such as necrotizing enteritis, microvillus inclusion disease or celic disease). The peptidomimetics of the present invention may also be utilized to increase the blood levels of high-density lipoprotein (HDL).

In addition, the conditions, diseases collectively referenced to as ‘Syndrome X’ or metabolic syndrome as detailed in Johannsson G., J., Clin. Endocrinol. Metab., 1997, 82, 727, may be treated employing the peptidomimetics of the invention. The peptidomimetics of the present invention may optionally be used in combination with suitable DPP-IV inhibitors for the treatment of some of the above disease states either by administering the compounds sequentially or as a formulation containing the peptidomimetics of the present invention along with suitable DPP-IV inhibitors.

No adverse effects were observed for any of the mentioned peptidomimetics of invention. The compounds of the present invention showed good glucose serum-lowering activity in the experimental animals used. These peptidomimetics are used for the testing/prophylaxis of diseases caused by hyperinsulinemia, hyperglycemia such as NIDDM, metabolic disorders, since such diseases are inter-linked to each other.

While the present invention has been described in terms of its specific embodiments, certain modifications and equivalents will be apparent to those skilled in the art and are intended to be included within the scope of the present invention.

1. An isolated peptidomimetic having a sequence of Formula (I),
A-B—C-D (I)
and a tautomeric form, and pharmaceutically acceptable salt, thereof wherein A represents a naturally or unnaturally occurring amino acid selected from Val (V), Lys (K), Leu (L), Ala (A), or Aib, or a dipeptide consisting of two amino acids selected from Val, Ala, Aib and Gly, operably linked to each other, such as Val-Val, Val-Ala, Val-Aib, Val-Gly, Gly-Val, Aib-Val, or Ala-Val;
B is defined by general Formula (II),
Z₁—Z₂—Z₃—Z₄—Z₅—Z₆—Z₇—Z₈—Z₉ (II)
wherein Z₁-Z₉ represents either the first N-terminal 1-9 residues of GLP-1 or glucagon peptide, or Z₁-Z₉ represents the suitably modified N-terminal 1-9 residues, wherein one or more of these residues are independently suitably replaced with a homolog of an unnatural or modified amino acid, wherein when any of Z₁-Z₉ represents a homolog of an unnatural or modified amino acid, then Z₁ represents Histidine (His; H) or α-methyl-Histidine (αMe-His); each of Z₂ and Z₄ independently represents a naturally or unnaturally occurring amino acid selected from Serine (Ser; S), Glycine (Gly; G), Alanine (Ala; A), α-methyl proline (αMe-Pro), α-amino-isobutyric acid (Aib), 1-amino cyclopropane carboxylic acid (AC₃C), and 1-amino-cyclopentanecarboxylic acid (AC₅C); 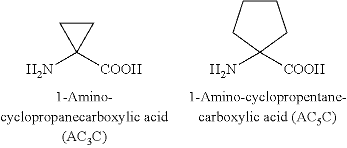
each of Z₃ and Z₉ independently represents a naturally or unnaturally occurring amino acid having an amino acid side chain comprising an acidic or amide group; each of Z₅, Z₇ and Z₈ independently represents a naturally or non-naturally occurring amino acid comprising a hydroxyl side chain; Z₆ represents a naturally or unnaturally occurring amino acid having a disubstituted alpha carbon having two side chains, wherein each of them may independently be an optionally substituted alkyl, aryl or an aralkyl group, wherein the substituents on the alkyl, aryl or aralkyl group is selected from one or more alkyl groups or one or more halo groups;
C represents a dipeptide of Formula III,
Z₁₀—Z₁₁ (III)
wherein Z₁₀ represents 2′-ethyl-4′-methoxy-biphenylalanine [Bip(OMe)] or α-methylated Bip(OMe) [αMe-Bip(OMe)]; 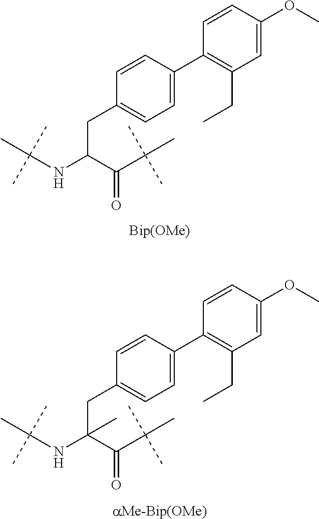
Z₁₁ represents 2-Amino-5-phenyl-pentanoic acid (APPA) or α-methylated APPA ((αMe-APPA); and 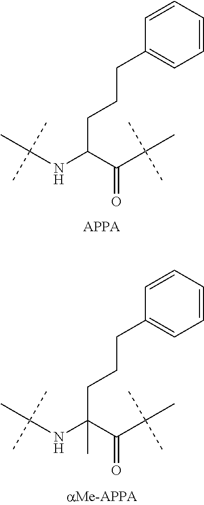
D represents a suitable C-terminus of peptidomimetics or a suitable amino acid.

2. The peptidomimetic of Formula (I) as claimed in claim 1, wherein Z₃ represents amino acids selected from Glutamine (Gln; Q), Glutamic acid (Glu; Q), Aspartic acid (Asp; D), alpha-methyl-Glutamine (αMe-Gln), alpha-methyl-Glutamic acid ((αMe-Glu), alpha-methyl-Aspartic acid (αMe-Asp), N-methyl-Glutamine (αMe-Gln), N-methyl-Glutamic acid (αMe-Glu), and N-methyl-Aspartic acid (αMe-Asp).

3. The peptidomimetic of Formula (I) as claimed in claim 1, wherein Z₉ represents amino acids selected from Glutamine (Gln; Q), Glutamic acid (Glu; Q), Aspartic acid (Asp; D), alpha-methyl-Glutamine (αMe-Gln), alpha-methyl-Glutamic acid (αMe-Glu), alpha-methyl-Aspartic acid (αMe-Asp), N-methyl-Glutamine (αMe-Gln), N-methyl-Glutamic acid (αMe-Glu), and N-methyl-Aspartic acid (αMe-Asp).

4. The peptidomimetic of Formula (I) as claimed in claim 1, wherein Z₅ is selected from Threonine (Thr; T), Serine (Ser; S), alpha-methyl-Threonine (αMe-Thr), alpha-methyl-Serine (αMe-Ser), Serine(OMe)-OH (Ser(OMe)), alpha-methyl-Serine(OMe)—OH (αMe-Ser(OMe)), N-methyl-Threonine (αMe-Thr), N-methyl-Serine (αMe-Ser), and 1-amino cyclopropane carboxylic acid (AC₃C).

5. The peptidomimetic of Formula (I) as claimed in claim 1, wherein Z₇ is selected from Threonine (Thr; T), Serine (Ser; S), alpha-methyl-Threonine (αMe-Thr), alpha-methyl-Serine (αMe-Ser), Serine(OMe)-OH (Ser(OMe)), alpha-methyl-Serine(OMe)-OH (αMe-Ser(OMe)), N-methyl-Threonine (αMe-Thr), N-methyl-Serine (αMe-Ser), and 1-amino cyclopropane carboxylic acid (AC₃C).

6. The peptidomimetic of Formula (I) as claimed in claim 1, wherein Z₈ is selected from Threonine (Thr; T), Serine (Ser; S), alpha-methyl-Threonine (αMe-Thr), alpha-methyl-Serine (αMe-Ser), Serine(OMe)-OH (Ser(OMe)), alpha-methyl-Serine(OMe)-OH (αMe-Ser(OMe)), N-methyl-Threonine (αMe-Thr), N-methyl-Serine (αMe-Ser), and 1-amino cyclopropane carboxylic acid (AC₃C).

7. The peptidomimetic of Formula (I) as claimed in claim 1, wherein Z₆ represents alpha-methyl-phenylalanine (-αMe-Phe-), alpha-methyl-2-fluorophenylalanine (-αMe-2F-Phe-) or alpha-methyl-2,6-difluorophenylalanine (-αMe-2,6-F-Phe-).

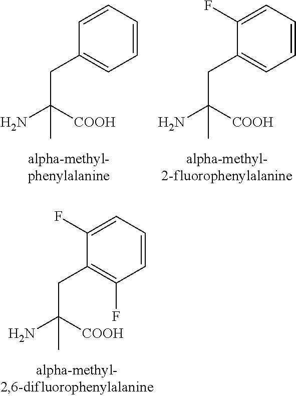

8. The peptidomimetic of Formula (I) as claimed in claim 1, wherein D represents the C-terminus of peptidomimetics selected from —COOH, —CONH₂, and —CH₂OH.

9. The peptidomimetic of Formula (I) as claimed in claim 1, wherein D represents a suitable amino acid selected from Val (V), Lys (K), Leu (L), Ala (A), and Aib.

10. The peptidomimetic of Formula (I) as claimed in claim 1, wherein
A represents a naturally or unnaturally occurring amino acid selected from Val (V), Lys (K), Leu (L), Ala (A), and Aib and a dipeptide consisting of two amino acids selected from Val, Ala, Aib and Gly, operably linked to each other, such as Val-Val, Val-Ala, Val-Aib, Val-Gly, Gly-Val, Aib-Val, and Ala-Val;
B represents the first N-terminal 1-9 residues of GLP-1 or glucagon peptide, defined by Formula (II),
Z₁—Z₂—Z₃—Z₄—Z₅—Z₆—Z₇—Z₈—Z₉ (II)
wherein, Z₁-Z₉ are optionally replaced with a homolog of an unnatural amino acid to improve its metabolic stability, wherein Z₁ represents Histidine (His; H) or α-methyl-Histidine (αMe-His); each of Z₂ and Z₄ independently represents a naturally or unnaturally occurring amino acid selected from Serine (Ser; S), Glycine (Gly; G), Alanine (Ala; A), α-methyl proline (αMe-Pro), α-amino-isobutyric acid (Aib), 1-amino cyclopropane carboxylic acid (AC₃C), and 1-amino-cyclopentanecarboxylic acid (AC₅C); 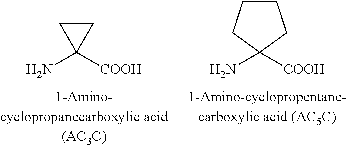
each of Z₃ and Z₉ independently represents a naturally or unnaturally occurring amino acid selected from Glutamine (Gln; Q), Glutamic acid (Glu; Q), Aspartic acid (Asp; D), alpha-methyl-Glutamine (αMe-Gln), alpha-methyl-Glutamic acid (αMe-Glu), alpha-methyl-Aspartic acid (αMe-Asp), N-methyl-Glutamine (αMe-Gln), N-methyl-Glutamic acid (αMe-Glu), and N-methyl-Aspartic acid (αMe-Asp); each of Z₅, Z₇ and Z₈ independently represents a naturally or non-naturally occurring amino acid selected from Threonine (Thr; T), Serine (Ser; S), alpha-methyl-Threonine (αMe-Thr), alpha-methyl-Serine (αMe-Ser), Serine(OMe)-OH (Ser(OMe)), alpha-methyl-Serine(OMe)-OH (αMe-Ser(OMe)), N-methyl-Threonine (αMe-Thr), N-methyl-Serine (αMe-Ser), or 1-amino cyclopropane carboxylic acid (AC₃C); Z₆ represents alpha-methyl-phenylalanine (-αMe-Phe-), alpha-methyl-2-fluorophenylalanine (-αMe-2F-Phe-) and alpha-methyl-2,6-difluorophenylalanine (-αMe-2,6-F-Phe-); 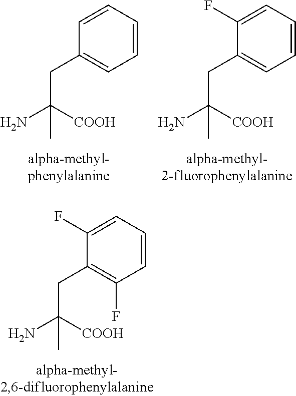
C represents a dipeptide of Formula III,
Z₁₀—Z₁₁ (III)
wherein Z₁₀ represents 2′-ethyl-4′-methoxy-biphenylalanine [Bip(OMe)] or α-methylated Bip(OMe) [αMe-Bip(OMe)]; 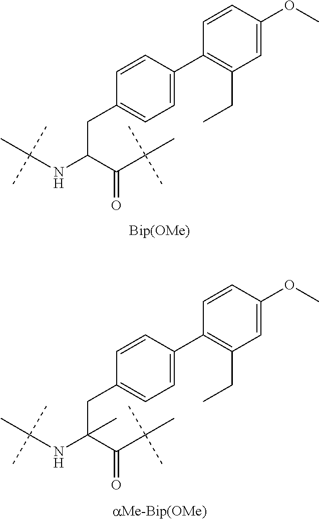
Z₁₁ represents 2-Amino-5-phenyl-pentanoic acid (APPA) or α-methylated APPA (αMe-APPA); and 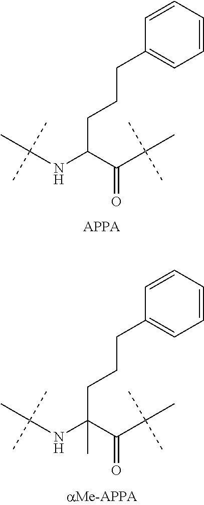
D represents a group selected from —COOH, —CONH₂, —CH₂OH and a suitable amino acid selected from Val (V), Lys (K), Leu (L), Ala (A), or Aib.

11. The peptidomimetic according to claim 1, wherein the aryl group is selected from phenyl, napthyl, indanyl, fluorenyl, and biphenyl groups.

12. A peptidomimetic of Formula (I) selected from:
VHSQGTFTSD-(αMe-Bip(OMe))-(APPA);
VHAQGTFTSD-(αMe-Bip(OMe))-(APPA);
VH-Aib-QGTFTSD-(αMe-Bip(OMe))-(APPA);
VH-(αMe-Pro)-QGTFTSD-(αMe-Bip(OMe))-(APPA);
VH-(AC₅C)-QGTFTSD-(αMe-Bip(OMe))-(APPA);
VH-(AC₃C)-QGTFTSD-(αMe-Bip(OMe))-(APPA);
VHSEGTFTSD-(αMe-Bip(OMe))-(APPA);
VHAEGTFTSD-(αMe-Bip(OMe))-(APPA);
VH-Aib-EGTFTSD-(αMe-Bip(OMe))-(APPA);
VH-(αMe-Pro)-EGTFTSD-(αMe-Bip(OMe))-(APPA);
VH-(AC₅C)-EGTFTSD-(αMe-Bip(OMe))-(APPA);
VH-(AC₃C)-EGTFTSD-(αMe-Bip(OMe))-(APPA);
VHSQGT-(αMe-2FPhe)-TSD-(αMe-Bip(OMe))-(APPA);
VHAQGT-(αMe-2FPhe)-TSD-(αMe-Bip(OMe))-(APPA);
VH-Aib-QGT-(αMe-2FPhe)-TSD-(αMe-Bip(OMe))-(APPA);
VH-(αMe-Pro)-QGT-(αMe-2FPhe)-TSD-(αMe-Bip(OMe))-(APPA);
VHSEGT-(αMe-2FPhe)-TSD-(αMe-Bip(OMe))-(APPA);
VHAEGT-(αMe-2FPhe)-TSD-(αMe-Bip(OMe))-(APPA);
VH-Aib-EGT-(αMe-2FPhe)-TSD-(αMe-Bip(OMe))-(APPA);
VH-(αMe-Pro)-EGT-(αMe-2FPhe)-TSD-(αMe-Bip(OMe))-(APPA);
VHSQGT-(αMe-2,6-diF-Phe)-TSD-(αMe-Bip(OMe))-(APPA);
VHAQGT-(αMe-2,6-diFPhe)-TSD-(αMe-Bip(OMe))-(APPA);
VH-Aib-QGT-(αMe-2,6-diFPhe)-TSD-(αMe-Bip(OMe))-(APPA);
VH-(αMe-Pro)-QGT-(αMe-2,6-diFPhe)-TSD-(αMe-Bip(OMe))-(APPA);
VHSEGT-(αMe-2,6-diFPhe)-TSD-(αMe-Bip(OMe))-(APPA);
VHAEGT-(αMe-2,6-diFPhe)-TSD-(αMe-Bip(OMe))-(APPA);
VH-Aib-EGT-(αMe-2,6-diFPhe)-TSD-(αMe-Bip(OMe))-(APPA);
VH-(αMe-Pro)-EGT-(αMe-2,6-diFPhe)-TSD-(αMe-Bip(OMe))-(APPA);
VHSQGTFT-(αMe-Ser)-D-(αMe-Bip(OMe))-(APPA);
VHAQGTFT-(αMe-Ser)-D-(αMe-Bip(OMe))-(APPA);
VH-Aib-QGTFT-(αMe-Ser)-D-(αMe-Bip(OMe))-(APPA);
VH-(αMe-Pro)-QGTFT-(αMe-Ser)-D-(αMe-Bip(OMe))-(APPA);
VHSEGTFT-(αMe-Ser)-D-(αMe-Bip(OMe))-(APPA);
VHAEGTFT-(αMe-Ser)-D-(αMe-Bip(OMe))-(APPA);
VH-Aib-EGTFT-(αMe-Ser)-D-(αMe-Bip(OMe))-(APPA);
VH-(αMe-Pro)-EGTFT-(αMe-Ser)-D-(αMe-Bip(OMe))-(APPA);
VHSQGT-(αMe-2FPhe)-T-(αMe-Ser)-D-(αMe-Bip(OMe))-(APPA);
VHAQGT-(αMe-2FPhe)-T-(αMe-Ser)-D-(αMe-Bip(OMe))-(APPA);
VH-Aib-QGT-(αMe-2FPhe)-T-(αMe-Ser)-D-(αMe-Bip(OMe))-(APPA);
VH-(αMe-Pro)-QGT-(αMe-2FPhe)-T-(αMe-Ser)-D-(αMe-Bip(OMe))-APPA);
VHSEGT-(αMe-2FPhe)-T-(αMe-Ser)-D-(αMe-Bip(OMe))-(APPA);
VHAEGT-(αMe-2FPhe)-T-(αMe-Ser)-D-(αMe-Bip(OMe))-(APPA);
VH-Aib-EGT-(αMe-2FPhe)-T-(αMe-Ser)-D-(αMe-Bip(OMe))-(APPA);
VH-(αMe-Pro)-EGT-(αMe-2FPhe)-T-(αMe-Ser)-D-(αMe-Bip(OMe))-APPA);
VHSQGT-(αMe-2,6-diF-Phe)-T-(αMe-Ser)-D-(αMe-Bip(OMe))-(APPA);
VHAQGT-(αMe-2,6-diFPhe)-T-(αMe-Ser)-D-(αMe-Bip(OMe))-(APPA);
VH-Aib-QGT-(αMe-2,6-diFPhe)-T-(αMe-Ser)-D-(αMe-Bip(OMe))-(APPA);
VH-(αMe-Pro)-QGT-(αMe-2,6-diFPhe)-T-(αMe-Ser)-D-(αMe-Bip(OMe))-(APPA);
VHSEGT-(αMe-2,6-diFPhe)-T-(αMe-Ser)-D-(αMe-Bip(OMe))-(APPA);
VHAEGT-(αMe-2,6-diFPhe)-T-(αMe-Ser)-D-(αMe-Bip(OMe))-(APPA);
VH-Aib-EGT-(αMe-2,6-diFPhe)-T-(αMe-Ser)-D-(αMe-Bip(OMe))-(APPA);
VH-(αMe-Pro)-EGT-(αMe-2,6-diFPhe)-T-(αMe-Ser)-D-(αMe-Bip(OMe))-(APPA);
VHSQGTFTS-(αMe-Asp)-(αMe-Bip(OMe))-(APPA);
VHAQGTFTS-(αMe-Asp)-(αMe-Bip(OMe))-(APPA);
VH-Aib-QGTFTS-(αMe-Asp)-(αMe-Bip(OMe))-(APPA);
VH-(αMe-Pro)-QGTFTS-(αMe-Asp)-(αMe-Bip(OMe))-(APPA);
VHSEGTFTS-(αMe-Asp)-(αMe-Bip(OMe))-(APPA);
VHAEGTFTS-(αMe-Asp)-(αMe-Bip(OMe))-(APPA);
VH-Aib-EGTFTS-(αMe-Asp)-(αMe-Bip(OMe))-(APPA);
VH-(αMe-Pro)-EGTFTS-(αMe-Asp)-(αMe-Bip(OMe))-(APPA);
VHSQGT-(αMe-2FPhe)-TS-(αMe-Asp)-(αMe-Bip(OMe))-(APPA);
VHAQGT-(αMe-2FPhe)-TS-(αMe-Asp)-(αMe-Bip(OMe))-(APPA);
VH-Aib-QGT-(αMe-2FPhe)-TS-(αMe-Asp)-(αMe-Bip(OMe))-(APPA);
VH-(αMe-Pro)-QGT-(αMe-2FPhe)-TS-(αMe-Asp)-(αMe-Bip(OMe))-(APPA);
VHSEGT-(αMe-2FPhe)-TS-(αMe-Asp)-(αMe-Bip(OMe))-(APPA);
VHAEGT-(αMe-2FPhe)-TS-(αMe-Asp)-(αMe-Bip(OMe))-(APPA);
VH-Aib-EGT-(αMe-2FPhe)-TS-(αMe-Asp)-(αMe-Bip(OMe))-(APPA);
VF-(αMe-Pro)-EGT-(αMe-2FPhe)-TS-(αMe-Asp)-(αMe-Bip(OMe))-(APPA);
VHSQGT-(αMe-2,6-diF-Phe)-TS-(αMe-Asp)-(αMe-Bip(OMe))-(APPA);
VHAQGT-(αMe-2,6-diFPhe)-TS-(αMe-Asp)-(αMe-Bip(OMe))-(APPA);
VH-Aib-QGT-(αMe-2,6-diFPhe)-TS-(αMe-Asp)-(αMe-Bip(OMe))-(APPA);
VH-(αMe-Pro)-QGT-(αMe-2,6-diFPhe)-TS-(αMe-Asp)-(αMe-Bip(OMe))-(APPA);
VHSEGT-(αMe-2,6-diFPhe)-TS-(αMe-Asp)-(αMe-Bip(OMe))-(APPA);
VHAEGT-(αMe-2,6-diFPhe)-TS-(αMe-Asp)-(αMe-Bip(OMe))-(APPA);
VH-Aib-EGT-(αMe-2,6-diFPhe)-TS-(αMe-Asp)-(αMe-Bip(OMe))-(APPA);
VH-(αMe-Pro)-EGT-(αMe-2,6-diFPhe)-TS-(αMe-Asp)-(αMe-Bip(OMe))-(APPA);
VHSQGTFTSD-Bip(OMe)-(αMe-APPA);
VHAQGTFTSD-Bip(OMe)-(αMe-APPA);
VH-Aib-QGTFTSD-B ip(OMe)-(αMe-APPA);
VH-(αMe-Pro)-QGTFTSD-Bip(OMe)-(αMe-APPA);
VH-(AC₅C)-QGTFTSD-Bip(OMe)-(αMe-APPA);
VH-(AC₃C)-QGTFTSD-Bip(OMe)-(αMe-APPA);
VHSEGTFTSD-Bip(OMe)-(αMe-APPA);
VHAEGTFTSD-Bip(OMe)-(αMe-APPA);
VH-Aib-EGTFTSD-Bip(OMe)-(αMe-APPA);
VH-(αMe-Pro)-EGTFTSD-Bip(OMe)-(αMe-APPA);
VHSQGT-(αMe-2FPhe)-TSD-Bip(OMe)-(αMe-APPA);
VHAQGT-(αMe-2FPhe)-TSD-Bip(OMe)-(αMe-APPA);
VH-Aib-QGT-(αMe-2FPhe)-TSD-Bip(OMe)-(αMe-APPA);
VH-(αMe-Pro)-QGT-(αMe-2FPhe)-TSD-Bip(OMe)-(αMe-APPA);
VHSEGT-(αMe-2FPhe)-TSD-Bip(OMe)-(αMe-APPA);
VHAEGT-(αMe-2FPhe)-TSD-Bip(OMe)-(αMe-APPA);
VH-Aib-EGT-(αMe-2FPhe)-TSD-Bip(OMe)-(αMe-APPA);
VH-(αMe-Pro)-EGT-(αMe-2FPhe)-TSD-Bip(OMe)-(αMe-APPA);
VHSQGT-(αMe-2,6-diF-Phe)-TSD-Bip(OMe)-(αMe-APPA);
VHAQGT-(αMe-2,6-diFPhe)-TSD-Bip(OMe)-(αMe-APPA);
VH-Aib-QGT-(αMe-2,6-diFPhe)-TSD-Bip(OMe)-(αMe-APPA);
VH-(αMe-Pro)-QGT-(αMe-2,6-diFPhe)-TSD-Bip(OMe)-(αMe-APPA);
VHSEGT-(αMe-2,6-diFPhe)-TSD-Bip(OMe)-(αMe-APPA);
VHAEGT-(αMe-2,6-diFPhe)-TSD-Bip(OMe)-(αMe-APPA);
VH-Aib-EGT-(αMe-2,6-diFPhe)-TSD-Bip(OMe)-(αMe-APPA);
VH-(αMe-Pro)-EGT-(αMe-2,6-diFPhe)-TSD-Bip(OMe)-(αMe-APPA);
VHSQGTFT-(αMe-Ser)-(αMe-Asp)-Bip(OMe)-(αMe-APPA);
VHAQGTFT-(αMe-Ser)-(αMe-Asp)-Bip(OMe)-(αMe-APPA);
VH-Aib-QGTFT-(αMe-Ser)-(αMe-Asp)-Bip(OMe)-(αMe-APPA);
VH-(αMe-Pro)-QGTFT-(αMe-Ser)-(αMe-Asp)-Bip(OMe)-(αMe-APPA);
VHSEGTFT-(αMe-Ser)-(αMe-Asp)-Bip(OMe)-(αMe-APPA);
VHAEGTFT-(αMe-Ser)-(αMe-Asp)-Bip(OMe)-(αMe-APPA);
VH-Aib-EGTFT-(αMe-Ser)-(αMe-Asp)-Bip(OMe)-(αMe-APPA);
VH-(αMe-Pro)-EGTFT-(αMe-Ser)-(αMe-Asp)-Bip(OMe)-(αMe-APPA);
VHSQGT-(αMe-2FPhe)-T-(αMe-Ser)-(αMe-Asp)-Bip(OMe)-(αMe-APPA);
VHAQGT-(αMe-2FPhe)-T-(αMe-Ser)-(αMe-Asp)-Bip(OMe)-(αMe-APPA);
VH-Aib-QGT-(αMe-2FPhe)-T-(αMe-Ser)-(αMe-Asp)-Bip(OMe)-(αMe-APPA);
VH-(αMe-Pro)-QGT-(αMe-2FPhe)-T-(αMe-Ser)-(αMe-Asp)-Bip(OMe)-(αMe-APPA);
VHSEGT-(αMe-2FPhe)-T-(αMe-Ser)-(αMe-Asp)-Bip(OMe)-(αMe-APPA);
VHAEGT-(αMe-2FPhe)-T-(αMe-Ser)-(αMe-Asp)-Bip(OMe)-(αMe-APPA);
VH-Aib-EGT-(αMe-2FPhe)-T-(αMe-Ser)-(αMe-Asp)-Bip(OMe)-(αMe-APPA);
VH-(αMe-Pro)-EGT-(αMe-2FPhe)-T-(αMe-Ser)-(αMe-Asp)-Bip(OMe)-(αMe-APPA);
VHSQGT-(αMe-2,6-diF-Phe)-T-(αMe-Ser)-(αMe-Asp)-Bip(OMe)-(αMe-APPA);
VHAQGT-(αMe-2,6-diFPhe)-T-(αMe-Ser)-(αMe-Asp)-Bip(OMe)-(αMe-APPA);
VH-Aib-QGT-(αMe-2,6-diFPhe)-T-(αMe-Ser)-(αMe-Asp)-Bip(OMe)-(αMe-APPA);
VH-(αMe-Pro)-QGT-(αMe-2,6-diFPhe)-T-(αMe-Ser)-(αMe-Asp)-Bip(OMe)-(αMe-APPA);
VHSEGT-(αMe-2,6-diFPhe)-T-(αMe-Ser)-(αMe-Asp)-Bip(OMe)-(αMe-APPA);
VHAEGT-(αMe-2,6-diFPhe)-T-(αMe-Ser)-(αMe-Asp)-Bip(OMe)-(αMe-APPA);
VH-Aib-EGT-(αMe-2,6-diFPhe)-T-(αMe-Ser)-(αMe-Asp)-Bip(OMe)-(αMe-APPA);
VH-(αMe-Pro)-EGT-(αMe-2,6-diFPhe)-T-(αMe-Ser)-(αMe-Asp)-Bip(OMe)-(αMe-APPA);
VHSQGTFTSD-(αMe-Bip(OMe))-(αMe-APPA);
VHAQGTFTSD-(αMe-Bip(OMe))-(αMe-APPA);
VH-Aib-QGTFTSD-(αMe-Bip(OMe))-(αMe-APPA);
VH-(αMe-Pro)-QGTFTSD-(αMe-Bip(OMe))-(αMe-APPA);
VH-(AC₅C)-QGTFTSD-(αMe-Bip(OMe))-(αMe-APPA);
VH-(AC₃C)-QGTFTSD-(αMe-Bip(OMe))-(αMe-APPA);
VHSEGTFTSD-(αMe-Bip(OMe))-(αMe-APPA);
VHAEGTFTSD-(αMe-Bip(OMe))-(αMe-APPA);
VH-Aib-EGTFTSD-(αMe-Bip(OMe))-(αMe-APPA);
VH-(αMe-Pro)-EGTFTSD-(αMe-Bip(OMe))-(αMe-APPA);
VHSQGT-(αMe-2FPhe)-TSD-(αMe-Bip(OMe))-(αMe-APPA);
VHAQGT-(αMe-2FPhe)-TSD-(αMe-Bip(OMe))-(αMe-APPA);
VH-Aib-QGT-(αMe-2FPhe)-TSD-(αMe-Bip(OMe))-(αMe-APPA);
VH-(αMe-Pro)-QGT-(αMe-2FPhe)-TSD-(αMe-Bip(OMe))-(αMe-APPA);
VHSEGT-(αMe-2FPhe)-TSD-(αMe-Bip(OMe))-(αMe-APPA);
VHAEGT-(αMe-2FPhe)-TSD-(αMe-Bip(OMe))-(αMe-APPA);
VH-Aib-EGT-(αMe-2FPhe)-TSD-(αMe-Bip(OMe))-(αMe-APPA);
VH-(αMe-Pro)-EGT-(αMe-2FPhe)-TSD-(αMe-Bip(OMe))-(αMe-APPA);
VHSQGT-(αMe-2,6-diF-Phe)-TSD-(αMe-Bip(OMe))-(αMe-APPA);
VHAQGT-(αMe-2,6-diFPhe)-TSD-(αMe-Bip(OMe))-(αMe-APPA);
VH-Aib-QGT-(αMe-2,6-diFPhe)-TSD-(αMe-Bip(OMe))-(αMe-APPA);
VH-(αMe-Pro)-QGT-(αMe-2,6-diFPhe)-TSD-(αMe-Bip(OMe))-(αMe-APPA);
VHSEGT-(αMe-2,6-diFPhe)-TSD-(αMe-Bip(OMe))-(αMe-APPA);
VHAEGT-(αMe-2,6-diFPhe)-TSD-(αMe-Bip(OMe))-(αMe-APPA);
VH-Aib-EGT-(αMe-2,6-diFPhe)-TSD-(αMe-Bip(OMe))-(αMe-APPA);
VH-(αMe-Pro)-EGT-(αMe-2 , 6-diFPhe )-TSD-(αMe-Bip (0Me))-(αMe-APPA) ;
VHSQGTFT-(αMe-Ser)-D-(αMe-Bip(OMe))-(αMe-APPA);
VHAQGTFT-(αMe-Ser)-D-(αMe-Bip(OMe))-(αMe-APPA);
VH-Aib-QGTFT-(αMe-Ser)-D-(αMe-Bip(OMe))-(αMe-APPA);
VH-(αMe-Pro)-QGTFT-(αMe-Ser)-D-(αMe-Bip(OMe))-(αMe-APPA);
VHSEGTFT-(αMe-Ser)-D-(αMe-Bip(OMe))-(αMe-APPA);
VHAEGTFT-(αMe-Ser)-D-(αMe-Bip(OMe))-(αMe-APPA);
VH-Aib-EGTFT-(αMe-Ser)-D-(αMe-Bip(OMe))-(αMe-APPA);
VH-(αMe-Pro)-EGTFT-(αMe-Ser)-D-(αMe-Bip(OMe))-(αMe-APPA);
VHSQGT-(αMe-2FPhe)-T-(αMe-Ser)-D-(αMe-Bip(OMe))-(αMe-APPA);
VHAQGT-(αMe-2FPhe)-T-(αMe-Ser)-D-(αMe-Bip(OMe))-(αMe-APPA);
VH-Aib-QGT-(αMe-2FPhe)-T-(αMe-Ser)-D-(αMe-Bip(OMe))-(αMe-APPA);
VH-(αMe-Pro)-QGT-(αMe-2FPhe)-T-(αMe-Ser)-D-(αMe-Bip(OMe))-(αMe-APPA);
VHSEGT-(αMe-2FPhe)-T-(αMe-Ser)-D-(αMe-Bip(OMe))-(αMe-APPA);
VHAEGT-(αMe-2FPhe)-T-(αMe-Ser)-D-(αMe-Bip(OMe))-(αMe-APPA);
VH-Aib-EGT-(αMe-2FPhe)-T-(αMe-Ser)-D-(αMe-Bip(OMe))-(αMe-APPA);
VH-(αMe-Pro)-EGT-(αMe-2FPhe)-T-(αMe-Ser)-D-(αMe-Bip(OMe))-(αMe-APPA);
VHSQGT-(αMe-2,6-diF-Phe)-T-(αMe-Ser)-D-(αMe-Bip(OMe))-(αMe-APPA);
VHAQGT-(αMe-2,6-diFPhe)-T-(αMe-Ser)-D-(αMe-Bip(OMe))-(αMe-APPA);
VH-Aib-QGT-(αMe-2,6-diFPhe)-T-(αMe-Ser)-D-(αMe-Bip(OMe))-(αMe-APPA);
VH-(αMe-Pro)-QGT-(αMe-2,6-diFPhe)-T-(αMe-Ser)-D-(αMe-Bip(OMe))-(αMe-APPA);
VHSEGT-(αMe-2,6-diFPhe)-T-(αMe-Ser)-D-(αMe-Bip(OMe))-(αMe-APPA);
VHAEGT-(αMe-2,6-diFPhe)-T-(αMe-Ser)-D-(αMe-Bip(OMe))-(αMe-APPA);
VH-Aib-EGT-(αMe-2,6-diFPhe)-T-(αMe-Ser)-D-(αMe-Bip(OMe))-(αMe-APPA);
VH-(αMe-Pro)-EGT-(αMe-2,6-diFPhe)-T-(αMe-Ser)-D-(αMe-Bip(OMe))-(αMe-APPA);
VHSQGTFTS-(αMe-Asp)-(αMe-Bip(OMe))-(αMe-APPA);
VHAQGTFTS-(αMe-Asp)-(αMe-Bip(OMe))-(αMe-APPA);
VH-Aib-QGTFTS-(αMe-Asp)-(αMe-Bip(OMe))-(αMe-APPA);
VH-(αMe-Pro)-QGTFTS-(αMe-Asp)-(αMe-Bip(OMe))-(αMe-APPA);
VHSEGTFTS-(αMe-Asp)-(αMe-Bip(OMe))-(αMe-APPA);
VHAEGTFTS-(αMe-Asp)-(αMe-Bip(OMe))-(αMe-APPA);
VH-Aib-EGTFTS-(αMe-Asp)-(αMe-Bip(OMe))-(αMe-APPA);
VH-(αMe-Pro)-EGTFTS-(αMe-Asp)-(αMe-Bip(OMe))-(αMe-APPA);
VHSQGT-(αMe-2FPhe)-TS-(αMe-Asp)-(αMe-Bip(OMe))-(αMe-APPA);
VHAQGT-(αMe-2FPhe)-TS-(αMe-Asp)-(αMe-Bip(OMe))-(αMe-APPA);
VH-Aib-QGT-(αMe-2FPhe)-TS-(αMe-Asp)-(αMe-Bip(OMe))-(αMe-APPA);
VH-(αMe-Pro)-QGT-(αMe-2FPhe)-TS-(αMe-Asp)-(αMe-Bip(OMe))-(αMe-APPA);
VHSEGT-(αMe-2FPhe)-TS-(αMe-Asp)-(αMe-Bip(OMe))-(αMe-APPA);
VHAEGT-(αMe-2FPhe)-TS-(αMe-Asp)-(αMe-Bip(OMe))-(αMe-APPA);
VH-Aib-EGT-(αMe-2FPhe)-TS-(αMe-Asp)-(αMe-Bip(OMe))-(αMe-APPA);
VH-(αMe-Pro)-EGT-(αMe-2FPhe)-TS-(αMe-Asp)-(αMe-Bip(OMe))-(αMe-APPA);
VHSQGT-(αMe-2,6-diF-Phe)-TS-(αMe-Asp)-(αMe-Bip(OMe))-(αMe-APPA);
VHAQGT-(αMe-2,6-diFPhe)-TS-(αMe-Asp)-(αMe-Bip(OMe))-(αMe-APPA);
VH-Aib-QGT-(αMe-2,6-diFPhe)-TS-(αMe-Asp)-(αMe-Bip(OMe))-(αMe-APPA);
VH-(αMe-Pro)-QGT-(αMe-2,6-diFPhe)-TS-(αMe-Asp)-(αMe-Bip(OMe))-(αMe-APPA);
VHSEGT-(αMe-2,6-diFPhe)-TS-(αMe-Asp)-(αMe-Bip(OMe))-(αMe-APPA);
VHAEGT-(αMe-2,6-diFPhe)-TS-(αMe-Asp)-(αMe-Bip(OMe))-(αMe-APPA);
VH-Aib-EGT-(αMe-2,6-diFPhe)-TS-(αMe-Asp)-(αMe-Bip(OMe))-(αMe-APPA);
VH-(αMe-Pro)-EGT-(αMe-2,6-diFPhe)-TS-(αMe-Asp)-(αMe-Bip(OMe))-(αMe-APPA);
VVHSQGTFTSD-(αMe-Bip(OMe))-(APPA);
VVHAQGTFTSD-(αMe-Bip(OMe))-(APPA);
VVH-Aib-QGTFTSD-(αMe-Bip(OMe))-(APPA);
VVH-(αMe-Pro)-QGTFTSD-(αMe-Bip(OMe))-(APPA);
VVHSEGTFTSD-(αMe-Bip(OMe))-(APPA);
VVHAEGTFTSD-(αMe-Bip(OMe))-(APPA);
VVH-Aib-EGTFTSD-(αMe-Bip(OMe))-(APPA);
VVH-(αMe-Pro)-EGTFTSD-(αMe-Bip(OMe))-(APPA);
VVHSQGT-(αMe-2FPhe)-TSD-(αMe-Bip(OMe))-(APPA);
VVHAQGT-(αMe-2FPhe)-TSD-(αMe-Bip(OMe))-(APPA);
VVH-Aib-QGT-(αMe-2FPhe)-TSD-(αMe-Bip(OMe))-(APPA);
VVH-(αMe-Pro)-QGT-(αMe-2FPhe)-TSD-(αMe-Bip(OMe))-(APPA);
VVHSEGT-(αMe-2FPhe)-TSD-(αMe-Bip(OMe))-(APPA);
VVHAEGT-(αMe-2FPhe)-TSD-(αMe-Bip(OMe))-(APPA);
VVH-Aib-EGT-(αMe-2FPhe)-TSD-(αMe-Bip(OMe))-(APPA);
VVH-(αMe-Pro)-EGT-(αMe-2FPhe)-TSD-(αMe-Bip(OMe))-(APPA);
VVHSQGT-(αMe-2,6-diF-Phe)-TSD-(αMe-Bip(OMe))-(APPA);
VVHAQGT-(αMe-2,6-diFPhe)-TSD-(αMe-Bip(OMe))-(APPA);
VVH-Aib-QGT-(αMe-2,6-diFPhe)-TSD-(αMe-Bip(OMe))-(APPA);
VVH-(αMe-Pro)-QGT-(αMe-2,6-diFPhe)-TSD-(αMe-Bip(OMe))-(APPA);
VVHSEGT-(αMe-2,6-diFPhe)-TSD-(αMe-Bip(OMe))-(APPA);
VVHAEGT-(αMe-2,6-diFPhe)-TSD-(αMe-Bip(OMe))-(APPA);
VVH-Aib-EGT-(αMe-2,6-diFPhe)-TSD-(αMe-Bip(OMe))-(APPA);
and
VVH-(αMe-Pro)-EGT-(αMe-2,6-diFPhe)-TSD-(αMe-Bip(OMe))-(APPA).

13. A pharmaceutical composition comprising a peptidomimetic of Formula (I) as defined in claim 1 and optionally one or more pharmaceutically acceptable carriers, excipients or diluents.

14. (canceled)

15. (canceled)

16. (canceled)

17. A method of preventing or treating diseases caused by hyperlipidaemia, hypercholesteremia, hyperglycemia, hyperinsulinemia, elevated blood levels of free fatty acids or glycerol, hypertriglyceridemia, wound healing, impaired glucose tolerance, leptin resistance, insulin resistance or other diabetic complications, comprising administering an effective, non-toxic amount of a peptidomimetic of Formula (I) as defined in claim 1 or a pharmaceutical composition thereof to a patient in need of such treatment.

18. (canceled)

19. (canceled)

20. A pharmaceutical composition comprising a peptidomimetic of Formula (I) as defined in claim 12 and optionally one or more pharmaceutically acceptable carriers, excipients or diluents.

21. The method of claim 17, wherein the peptidomimetic is an agonist of GLP-1 receptor.

22. The method of claim 17, wherein the peptidomimetic is an antagonist of glucagon receptor.


SEQUENCE LISTING (228 sequences)

SEQ ID NO: 1 - protein, 29 aa, Homo sapiens
  misc_feature (1)..(1): This is N-terminal of GLUCAGON peptide
HSQGTFTSDYSKYLDSRRAQDFVQWLMNT

SEQ ID NO: 2 - protein, 30 aa, Homo sapiens
  misc_feature (1)..(1): This is N-terminal of GLP-1 (7-36) peptide
HAEGTFTSDVSSYLEGQAAKEFIAWLVKGR

SEQ ID NO: 3 - protein, 9 aa, Homo sapiens
  misc_feature (1)..(9): These are First N-terminal 1-9 residues of GLP-1 peptide
HAEGTFTSD

SEQ ID NO: 4 - protein, 9 aa, Homo sapiens
  misc_feature (1)..(9): These are First N-terminal 1-9 residues of Glucagon peptide
HSQGTFTSD

SEQ ID NO: 5 - protein, 12 aa, Artificial
  This peptidomimetic was synthetically generated and prepared
VHSQGTFTSDFF

SEQ ID NO: 6 - protein, 12 aa, Artificial
  This peptidomimetic was synthetically generated and prepared
VHAQGTFTSDFF

SEQ ID NO: 7 - protein, 12 aa, Artificial
  This peptidomimetic was synthetically generated and prepared
VHAQGTFTSDFF

SEQ ID NO: 8 - protein, 12 aa, Artificial
  This peptidomimetic was synthetically generated and prepared
VHPQGTFTSDFF

SEQ ID NO: 9 - protein, 12 aa, Artificial
  This peptidomimetic was synthetically generated and prepared
VHAQGTFTSDFF

SEQ ID NO: 10 - protein, 12 aa, Artificial
  This peptidomimetic was synthetically generated and prepared
VHAQGTFTSDFF

SEQ ID NO: 11 - protein, 12 aa, Artificial
  This peptidomimetic was synthetically generated and prepared
VHSEGTFTSDFF

SEQ ID NO: 12 - protein, 12 aa, Artificial
  This peptidomimetic was synthetically generated and prepared
VHAEGTFTSDFF

SEQ ID NO: 13 - protein, 12 aa, Artificial
  This peptidomimetic was synthetically generated and prepared
VHAEGTFTSDFF

SEQ ID NO: 14 - protein, 12 aa, Artificial
  This peptidomimetic was synthetically generated and prepared
VHPEGTFTSDFF

SEQ ID NO: 15 - protein, 12 aa, Artificial
  This peptidomimetic was synthetically generated and prepared
VHAEGTFTSDFF

SEQ ID NO: 16 - protein, 12 aa, Artificial
  This peptidomimetic was synthetically generated and prepared
VHAEGTFTSDFF

SEQ ID NO: 17 - protein, 12 aa, Artificial
  This peptidomimetic was synthetically generated and prepared
VHSQGTFTSDFF

SEQ ID NO: 18 - protein, 12 aa, Artificial
  This peptidomimetic was synthetically generated and prepared
VHAQGTFTSDFF

SEQ ID NO: 19 - protein, 12 aa, Artificial
  This peptidomimetic was synthetically generated and prepared
VHAQGTFTSDFF

SEQ ID NO: 20 - protein, 12 aa, Artificial
  This peptidomimetic was synthetically generated and prepared
VHPQGTFTSDFF

SEQ ID NO: 21 - protein, 12 aa, Artificial
  This peptidomimetic was synthetically generated and prepared
VHSEGTFTSDFF

SEQ ID NO: 22 - protein, 12 aa, Artificial
  This peptidomimetic was synthetically generated and prepared
VHAEGTFTSDFF

SEQ ID NO: 23 - protein, 12 aa, Artificial
  This peptidomimetic was synthetically generated and prepared
VHAEGTFTSDFF

SEQ ID NO: 24 - protein, 12 aa, Artificial
  This peptidomimetic was synthetically generated and prepared
VHPEGTFTSDFF

SEQ ID NO: 25 - protein, 12 aa, Artificial
  This peptidomimetic was synthetically generated and prepared
VHSQGTFTSDFF

SEQ ID NO: 26 - protein, 12 aa, Artificial
  This peptidomimetic was synthetically generated and prepared
VHAQGTFTSDFF

SEQ ID NO: 27 - protein, 12 aa, Artificial
  This peptidomimetic was synthetically generated and prepared
VHAQGTFTSDFF

SEQ ID NO: 28 - protein, 12 aa, Artificial
  This peptidomimetic was synthetically generated and prepared
VHPQGTFTSDFF

SEQ ID NO: 29 - protein, 12 aa, Artificial
  This peptidomimetic was synthetically generated and prepared
VHSEGTFTSDFF

SEQ ID NO: 30 - protein, 12 aa, Artificial
  This peptidomimetic was synthetically generated and prepared
VHAEGTFTSDFF

SEQ ID NO: 31 - protein, 12 aa, Artificial
  This peptidomimetic was synthetically generated and prepared
VHAEGTFTSDFF

SEQ ID NO: 32 - protein, 12 aa, Artificial
  This peptidomimetic was synthetically generated and prepared
VHPEGTFTSDFF

SEQ ID NO: 33 - protein, 12 aa, Artificial
  This peptidomimetic was synthetically generated and prepared
VHSQGTFTSDFF

SEQ ID NO: 34 - protein, 12 aa, Artificial
  This peptidomimetic was synthetically generated and prepared
VHAQGTFTSDFF

SEQ ID NO: 35 - protein, 12 aa, Artificial
  This peptidomimetic was synthetically generated and prepared
VHAQGTFTSDFF

SEQ ID NO: 36 - protein, 12 aa, Artificial
  This peptidomimetic was synthetically generated and prepared
VHPQGTFTSDFF

SEQ ID NO: 37 - protein, 12 aa, Artificial
  This peptidomimetic was synthetically generated and prepared
VHSEGTFTSDFF

SEQ ID NO: 38 - protein, 12 aa, Artificial
  This peptidomimetic was synthetically generated and prepared
VHAEGTFTSDFF

SEQ ID NO: 39 - protein, 12 aa, Artificial
  This peptidomimetic was synthetically generated and prepared
VHAEGTFTSDFF

SEQ ID NO: 40 - protein, 12 aa, Artificial
  This peptidomimetic was synthetically generated and prepared
VHPEGTFTSDFF

SEQ ID NO: 41 - protein, 12 aa, Artificial
  This peptidomimetic was synthetically generated and prepared
VHSQGTFTSDFF

SEQ ID NO: 42 - protein, 12 aa, Artificial
  This peptidomimetic was synthetically generated and prepared
VHAQGTFTSDFF

SEQ ID NO: 43 - protein, 12 aa, Artificial
  This peptidomimetic was synthetically generated and prepared
VHAQGTFTSDFF

SEQ ID NO: 44 - protein, 12 aa, Artificial
  This peptidomimetic was synthetically generated and prepared
VHPQGTFTSDFF

SEQ ID NO: 45 - protein, 12 aa, Artificial
  This peptidomimetic was synthetically generated and prepared
VHSEGTFTSDFF

SEQ ID NO: 46 - protein, 12 aa, Artificial
  This peptidomimetic was synthetically generated and prepared
VHAEGTFTSDFF

SEQ ID NO: 47 - protein, 12 aa, Artificial
  This peptidomimetic was synthetically generated and prepared
VHAEGTFTSDFF

SEQ ID NO: 48 - protein, 12 aa, Artificial
  This peptidomimetic was synthetically generated and prepared
VHPEGTFTSDFF

SEQ ID NO: 49 - protein, 12 aa, Artificial
  This peptidomimetic was synthetically generated and prepared
VHSQGTFTSDFF

SEQ ID NO: 50 - protein, 12 aa, Artificial
  This peptidomimetic was synthetically generated and prepared
VHAQGTFTSDFF

SEQ ID NO: 51 - protein, 12 aa, Artificial
  This peptidomimetic was synthetically generated and prepared
VHAQGTFTSDFF

SEQ ID NO: 52 - protein, 12 aa, Artificial
  This peptidomimetic was synthetically generated and prepared
VHPQGTFTSDFF

SEQ ID NO: 53 - protein, 12 aa, Artificial
  This peptidomimetic was synthetically generated and prepared
VHSEGTFTSDFF

SEQ ID NO: 54 - protein, 12 aa, Artificial
  This peptidomimetic was synthetically generated and prepared
VHAEGTFTSDFF

SEQ ID NO: 55 - protein, 12 aa, Artificial
  This peptidomimetic was synthetically generated and prepared
VHAEGTFTSDFF

SEQ ID NO: 56 - protein, 12 aa, Artificial
  This peptidomimetic was synthetically generated and prepared
VHPEGTFTSDFF

SEQ ID NO: 57 - protein, 12 aa, Artificial
  This peptidomimetic was synthetically generated and prepared
VHSQGTFTSDFF

SEQ ID NO: 58 - protein, 12 aa, Artificial
  This peptidomimetic was synthetically generated and prepared
VHAQGTFTSDFF

SEQ ID NO: 59 - protein, 12 aa, Artificial
  This peptidomimetic was synthetically generated and prepared
VHAQGTFTSDFF

SEQ ID NO: 60 - protein, 12 aa, Artificial
  This peptidomimetic was synthetically generated and prepared
VHPQGTFTSDFF

SEQ ID NO: 61 - protein, 12 aa, Artificial
  This peptidomimetic was synthetically generated and prepared
VHSEGTFTSDFF

SEQ ID NO: 62 - protein, 12 aa, Artificial
  This peptidomimetic was synthetically generated and prepared
VHAEGTFTSDFF

SEQ ID NO: 63 - protein, 12 aa, Artificial
  This peptidomimetic was synthetically generated and prepared
VHAEGTFTSDFF

SEQ ID NO: 64 - protein, 12 aa, Artificial
  This peptidomimetic was synthetically generated and prepared
VHPEGTFTSDFF

SEQ ID NO: 65 - protein, 12 aa, Artificial
  This peptidomimetic was synthetically generated and prepared
VHSQGTFTSDFF

SEQ ID NO: 66 - protein, 12 aa, Artificial
  This peptidomimetic was synthetically generated and prepared
VHAQGTFTSDFF

SEQ ID NO: 67 - protein, 12 aa, Artificial
  This peptidomimetic was synthetically generated and prepared
VHAQGTFTSDFF

SEQ ID NO: 68 - protein, 12 aa, Artificial
  This peptidomimetic was synthetically generated and prepared
VHPQGTFTSDFF

SEQ ID NO: 69 - protein, 12 aa, Artificial
  This peptidomimetic was synthetically generated and prepared
VHSEGTFTSDFF

SEQ ID NO: 70 - protein, 12 aa, Artificial
  This peptidomimetic was synthetically generated and prepared
VHAEGTFTSDFF

SEQ ID NO: 71 - protein, 12 aa, Artificial
  This peptidomimetic was synthetically generated and prepared
VHAEGTFTSDFF

SEQ ID NO: 72 - protein, 12 aa, Artificial
  This peptidomimetic was synthetically generated and prepared
VHPEGTFTSDFF

SEQ ID NO: 73 - protein, 12 aa, Artificial
  This peptidomimetic was synthetically generated and prepared
VHSQGTFTSDFF

SEQ ID NO: 74 - protein, 12 aa, Artificial
  This peptidomimetic was synthetically generated and prepared
VHAQGTFTSDFF

SEQ ID NO: 75 - protein, 12 aa, Artificial
  This peptidomimetic was synthetically generated and prepared
VHAQGTFTSDFF

SEQ ID NO: 76 - protein, 12 aa, Artificial
  This peptidomimetic was synthetically generated and prepared
VHPQGTFTSDFF

SEQ ID NO: 77 - protein, 12 aa, Artificial
  This peptidomimetic was synthetically generated and prepared
VHSEGTFTSDFF

SEQ ID NO: 78 - protein, 12 aa, Artificial
  This peptidomimetic was synthetically generated and prepared
VHAEGTFTSDFF

SEQ ID NO: 79 - protein, 12 aa, Artificial
  This peptidomimetic was synthetically generated and prepared
VHAEGTFTSDFF

SEQ ID NO: 80 - protein, 12 aa, Artificial
  This peptidomimetic was synthetically generated and prepared
VHPEGTFTSDFF

SEQ ID NO: 81 - protein, 12 aa, Artificial
  This peptidomimetic was synthetically generated and prepared
VHSQGTFTSDFF

SEQ ID NO: 82 - protein, 12 aa, Artificial
  This peptidomimetic was synthetically generated and prepared
VHAQGTFTSDFF

SEQ ID NO: 83 - protein, 12 aa, Artificial
  This peptidomimetic was synthetically generated and prepared
VHAQGTFTSDFF

SEQ ID NO: 84 - protein, 12 aa, Artificial
  This peptidomimetic was synthetically generated and prepared
VHPQGTFTSDFF

SEQ ID NO: 85 - protein, 12 aa, Artificial
  This peptidomimetic was synthetically generated and prepared
VHAQGTFTSDFF

SEQ ID NO: 86 - protein, 12 aa, Artificial
  This peptidomimetic was synthetically generated and prepared
VHAQGTFTSDFF

SEQ ID NO: 87 - protein, 12 aa, Artificial
  This peptidomimetic was synthetically generated and prepared
VHSEGTFTSDFF

SEQ ID NO: 88 - protein, 12 aa, Artificial
  This peptidomimetic was synthetically generated and prepared
VHAEGTFTSDFF

SEQ ID NO: 89 - protein, 12 aa, Artificial
  This peptidomimetic was synthetically generated and prepared
VHAEGTFTSDFF

SEQ ID NO: 90 - protein, 12 aa, Artificial
  This peptidomimetic was synthetically generated and prepared
VHPEGTFTSDFF

SEQ ID NO: 91 - protein, 12 aa, Artificial
  This peptidomimetic was synthetically generated and prepared
VHSQGTFTSDFF

SEQ ID NO: 92 - protein, 12 aa, Artificial
  This peptidomimetic was synthetically generated and prepared
VHAQGTFTSDFF

SEQ ID NO: 93 - protein, 12 aa, Artificial
  This peptidomimetic was synthetically generated and prepared
VHAQGTFTSDFF

SEQ ID NO: 94 - protein, 12 aa, Artificial
  This peptidomimetic was synthetically generated and prepared
VHPQGTFTSDFF

SEQ ID NO: 95 - protein, 12 aa, Artificial
  This peptidomimetic was synthetically generated and prepared
VHSEGTFTSDFF

SEQ ID NO: 96 - protein, 12 aa, Artificial
  This peptidomimetic was synthetically generated and prepared
VHAEGTFTSDFF

SEQ ID NO: 97 - protein, 12 aa, Artificial
  This peptidomimetic was synthetically generated and prepared
VHAEGTFTSDFF

SEQ ID NO: 98 - protein, 12 aa, Artificial
  This peptidomimetic was synthetically generated and prepared
VHPEGTFTSDFF

SEQ ID NO: 99 - protein, 12 aa, Artificial
  This peptidomimetic was synthetically generated and prepared
VHSQGTFTSDFF

SEQ ID NO: 100 - protein, 12 aa, Artificial
  This peptidomimetic was synthetically generated and prepared
VHAQGTFTSDFF

SEQ ID NO: 101 - protein, 12 aa, Artificial
  This peptidomimetic was synthetically generated and prepared
VHAQGTFTSDFF

SEQ ID NO: 102 - protein, 12 aa, Artificial
  This peptidomimetic was synthetically generated and prepared
VHPQGTFTSDFF

SEQ ID NO: 103 - protein, 12 aa, Artificial
  This peptidomimetic was synthetically generated and prepared
VHSEGTFTSDFF

SEQ ID NO: 104 - protein, 12 aa, Artificial
  This peptidomimetic was synthetically generated and prepared
VHAEGTFTSDFF

SEQ ID NO: 105 - protein, 12 aa, Artificial
  This peptidomimetic was synthetically generated and prepared
VHAEGTFTSDFF

SEQ ID NO: 106 - protein, 12 aa, Artificial
  This peptidomimetic was synthetically generated and prepared
VHPEGTFTSDFF

SEQ ID NO: 107 - protein, 12 aa, Artificial
  This peptidomimetic was synthetically generated and prepared
VHSQGTFTSDFF

SEQ ID NO: 108 - protein, 12 aa, Artificial
  This peptidomimetic was synthetically generated and prepared
VHAQGTFTSDFF

SEQ ID NO: 109 - protein, 12 aa, Artificial
  This peptidomimetic was synthetically generated and prepared
VHAQGTFTSDFF

SEQ ID NO: 110 - protein, 12 aa, Artificial
  This peptidomimetic was synthetically generated and prepared
VHPQGTFTSDFF

SEQ ID NO: 111 - protein, 12 aa, Artificial
  This peptidomimetic was synthetically generated and prepared
VHSEGTFTSDFF

SEQ ID NO: 112 - protein, 12 aa, Artificial
  This peptidomimetic was synthetically generated and prepared
VHAEGTFTSDFF

SEQ ID NO: 113 - protein, 12 aa, Artificial
  This peptidomimetic was synthetically generated and prepared
VHAEGTFTSDFF

SEQ ID NO: 114 - protein, 12 aa, Artificial
  This peptidomimetic was synthetically generated and prepared
VHPEGTFTSDFF

SEQ ID NO: 115 - protein, 12 aa, Artificial
  This peptidomimetic was synthetically generated and prepared
VHSQGTFTSDFF

SEQ ID NO: 116 - protein, 12 aa, Artificial
  This peptidomimetic was synthetically generated and prepared
VHAQGTFTSDFF

SEQ ID NO: 117 - protein, 12 aa, Artificial
  This peptidomimetic was synthetically generated and prepared
VHAQGTFTSDFF

SEQ ID NO: 118 - protein, 12 aa, Artificial
  This peptidomimetic was synthetically generated and prepared
VHPQGTFTSDFF

SEQ ID NO: 119 - protein, 12 aa, Artificial
  This peptidomimetic was synthetically generated and prepared
VHSEGTFTSDFF

SEQ ID NO: 120 - protein, 12 aa, Artificial
  This peptidomimetic was synthetically generated and prepared
VHAEGTFTSDFF

SEQ ID NO: 121 - protein, 12 aa, Artificial
  This peptidomimetic was synthetically generated and prepared
VHAEGTFTSDFF

SEQ ID NO: 122 - protein, 12 aa, Artificial
  This peptidomimetic was synthetically generated and prepared
VHPEGTFTSDFF

SEQ ID NO: 123 - protein, 12 aa, Artificial
  This peptidomimetic was synthetically generated and prepared
VHSQGTFTSDFF

SEQ ID NO: 124 - protein, 12 aa, Artificial
  This peptidomimetic was synthetically generated and prepared
VHAQGTFTSDFF

SEQ ID NO: 125 - protein, 12 aa, Artificial
  This peptidomimetic was synthetically generated and prepared
VHAQGTFTSDFF

SEQ ID NO: 126 - protein, 12 aa, Artificial
  This peptidomimetic was synthetically generated and prepared
VHPQGTFTSDFF

SEQ ID NO: 127 - protein, 12 aa, Artificial
  This peptidomimetic was synthetically generated and prepared
VHSEGTFTSDFF

SEQ ID NO: 128 - protein, 12 aa, Artificial
  This peptidomimetic was synthetically generated and prepared
VHAEGTFTSDFF

SEQ ID NO: 129 - protein, 12 aa, Artificial
  This peptidomimetic was synthetically generated and prepared
VHAEGTFTSDFF

SEQ ID NO: 130 - protein, 12 aa, Artificial
  This peptidomimetic was synthetically generated and prepared
VHPEGTFTSDFF

SEQ ID NO: 131 - protein, 12 aa, Artificial
  This peptidomimetic was synthetically generated and prepared
VHSQGTFTSDFF

SEQ ID NO: 132 - protein, 12 aa, Artificial
  This peptidomimetic was synthetically generated and prepared
VHAQGTFTSDFF

SEQ ID NO: 133 - protein, 12 aa, Artificial
  This peptidomimetic was synthetically generated and prepared
VHAQGTFTSDFF

SEQ ID NO: 134 - protein, 12 aa, Artificial
  This peptidomimetic was synthetically generated and prepared
VHPQGTFTSDFF

SEQ ID NO: 135 - protein, 12 aa, Artificial
  This peptidomimetic was synthetically generated and prepared
VHAQGTFTSDFF

SEQ ID NO: 136 - protein, 12 aa, Artificial
  This peptidomimetic was synthetically generated and prepared
VHAQGTFTSDFF

SEQ ID NO: 137 - protein, 12 aa, Artificial
  This peptidomimetic was synthetically generated and prepared
VHSEGTFTSDFF

SEQ ID NO: 138 - protein, 12 aa, Artificial
  This peptidomimetic was synthetically generated and prepared
VHAEGTFTSDFF

SEQ ID NO: 139 - protein, 12 aa, Artificial
  This peptidomimetic was synthetically generated and prepared
VHAEGTFTSDFF

SEQ ID NO: 140 - protein, 12 aa, Artificial
  This peptidomimetic was synthetically generated and prepared
VHPEGTFTSDFF

SEQ ID NO: 141 - protein, 12 aa, Artificial
  This peptidomimetic was synthetically generated and prepared
VHSQGTFTSDFF

SEQ ID NO: 142 - protein, 12 aa, Artificial
  This peptidomimetic was synthetically generated and prepared
VHAQGTFTSDFF

SEQ ID NO: 143 - protein, 12 aa, Artificial
  This peptidomimetic was synthetically generated and prepared
VHAQGTFTSDFF

SEQ ID NO: 144 - protein, 12 aa, Artificial
  This peptidomimetic was synthetically generated and prepared
VHPQGTFTSDFF

SEQ ID NO: 145 - protein, 12 aa, Artificial
  This peptidomimetic was synthetically generated and prepared
VHSEGTFTSDFF

SEQ ID NO: 146 - protein, 12 aa, Artificial
  This peptidomimetic was synthetically generated and prepared
VHAEGTFTSDFF

SEQ ID NO: 147 - protein, 12 aa, Artificial
  This peptidomimetic was synthetically generated and prepared
VHAEGTFTSDFF

SEQ ID NO: 148 - protein, 12 aa, Artificial
  This peptidomimetic was synthetically generated and prepared
VHPEGTFTSDFF

SEQ ID NO: 149 - protein, 12 aa, Artificial
  This peptidomimetic was synthetically generated and prepared
VHSQGTFTSDFF

SEQ ID NO: 150 - protein, 12 aa, Artificial
  This peptidomimetic was synthetically generated and prepared
VHAQGTFTSDFF

SEQ ID NO: 151 - protein, 12 aa, Artificial
  This peptidomimetic was synthetically generated and prepared
VHAQGTFTSDFF

SEQ ID NO: 152 - protein, 12 aa, Artificial
  This peptidomimetic was synthetically generated and prepared
VHPQGTFTSDFF

SEQ ID NO: 153 - protein, 12 aa, Artificial
  This peptidomimetic was synthetically generated and prepared
VHSEGTFTSDFF

SEQ ID NO: 154 - protein, 12 aa, Artificial
  This peptidomimetic was synthetically generated and prepared
VHAEGTFTSDFF

SEQ ID NO: 155 - protein, 12 aa, Artificial
  This peptidomimetic was synthetically generated and prepared
VHAEGTFTSDFF

SEQ ID NO: 156 - protein, 12 aa, Artificial
  This peptidomimetic was synthetically generated and prepared
VHPEGTFTSDFF

SEQ ID NO: 157 - protein, 12 aa, Artificial
  This peptidomimetic was synthetically generated and prepared
VHSQGTFTSDFF

SEQ ID NO: 158 - protein, 12 aa, Artificial
  This peptidomimetic was synthetically generated and prepared
VHAQGTFTSDFF

SEQ ID NO: 159 - protein, 12 aa, Artificial
  This peptidomimetic was synthetically generated and prepared
VHAQGTFTSDFF

SEQ ID NO: 160 - protein, 12 aa, Artificial
  This peptidomimetic was synthetically generated and prepared
VHPQGTFTSDFF

SEQ ID NO: 161 - protein, 12 aa, Artificial
  This peptidomimetic was synthetically generated and prepared
VHSEGTFTSDFF

SEQ ID NO: 162 - protein, 12 aa, Artificial
  This peptidomimetic was synthetically generated and prepared
VHAEGTFTSDFF

SEQ ID NO: 163 - protein, 12 aa, Artificial
  This peptidomimetic was synthetically generated and prepared
VHAEGTFTSDFF

SEQ ID NO: 164 - protein, 12 aa, Artificial
  This peptidomimetic was synthetically generated and prepared
VHPEGTFTSDFF

SEQ ID NO: 165 - protein, 12 aa, Artificial
  This peptidomimetic was synthetically generated and prepared
VHSQGTFTSDFF

SEQ ID NO: 166 - protein, 12 aa, Artificial
  This peptidomimetic was synthetically generated and prepared
VHAQGTFTSDFF

SEQ ID NO: 167 - protein, 12 aa, Artificial
  This peptidomimetic was synthetically generated and prepared
VHAQGTFTSDFF

SEQ ID NO: 168 - protein, 12 aa, Artificial
  This peptidomimetic was synthetically generated and prepared
VHPQGTFTSDFF

SEQ ID NO: 169 - protein, 12 aa, Artificial
  This peptidomimetic was synthetically generated and prepared
VHSEGTFTSDFF

SEQ ID NO: 170 - protein, 12 aa, Artificial
  This peptidomimetic was synthetically generated and prepared
VHAEGTFTSDFF

SEQ ID NO: 171 - protein, 12 aa, Artificial
  This peptidomimetic was synthetically generated and prepared
VHAEGTFTSDFF

SEQ ID NO: 172 - protein, 12 aa, Artificial
  This peptidomimetic was synthetically generated and prepared
VHPEGTFTSDFF

SEQ ID NO: 173 - protein, 12 aa, Artificial
  This peptidomimetic was synthetically generated and prepared
VHSQGTFTSDFF

SEQ ID NO: 174 - protein, 12 aa, Artificial
  This peptidomimetic was synthetically generated and prepared
VHAQGTFTSDFF

SEQ ID NO: 175 - protein, 12 aa, Artificial
  This peptidomimetic was synthetically generated and prepared
VHAQGTFTSDFF

SEQ ID NO: 176 - protein, 12 aa, Artificial
  This peptidomimetic was synthetically generated and prepared
VHPQGTFTSDFF

SEQ ID NO: 177 - protein, 12 aa, Artificial
  This peptidomimetic was synthetically generated and prepared
VHSEGTFTSDFF

SEQ ID NO: 178 - protein, 12 aa, Artificial
  This peptidomimetic was synthetically generated and prepared
VHAEGTFTSDFF

SEQ ID NO: 179 - protein, 12 aa, Artificial
  This peptidomimetic was synthetically generated and prepared
VHAEGTFTSDFF

SEQ ID NO: 180 - protein, 12 aa, Artificial
  This peptidomimetic was synthetically generated and prepared
VHPEGTFTSDFF

SEQ ID NO: 181 - protein, 12 aa, Artificial
  This peptidomimetic was synthetically generated and prepared
VHSQGTFTSDFF

SEQ ID NO: 182 - protein, 12 aa, Artificial
  This peptidomimetic was synthetically generated and prepared
VHAQGTFTSDFF

SEQ ID NO: 183 - protein, 12 aa, Artificial
  This peptidomimetic was synthetically generated and prepared
VHAQGTFTSDFF

SEQ ID NO: 184 - protein, 12 aa, Artificial
  This peptidomimetic was synthetically generated and prepared
VHPQGTFTSDFF

SEQ ID NO: 185 - protein, 12 aa, Artificial
  This peptidomimetic was synthetically generated and prepared
VHSEGTFTSDFF

SEQ ID NO: 186 - protein, 12 aa, Artificial
  This peptidomimetic was synthetically generated and prepared
VHAEGTFTSDFF

SEQ ID NO: 187 - protein, 12 aa, Artificial
  This peptidomimetic was synthetically generated and prepared
VHAEGTFTSDFF

SEQ ID NO: 188 - protein, 12 aa, Artificial
  This peptidomimetic was synthetically generated and prepared
VHPEGTFTSDFF

SEQ ID NO: 189 - protein, 12 aa, Artificial
  This peptidomimetic was synthetically generated and prepared
VHSQGTFTSDFF

SEQ ID NO: 190 - protein, 12 aa, Artificial
  This peptidomimetic was synthetically generated and prepared
VHAQGTFTSDFF

SEQ ID NO: 191 - protein, 12 aa, Artificial
  This peptidomimetic was synthetically generated and prepared
VHAQGTFTSDFF

SEQ ID NO: 192 - protein, 12 aa, Artificial
  This peptidomimetic was synthetically generated and prepared
VHPQGTFTSDFF

SEQ ID NO: 193 - protein, 12 aa, Artificial
  This peptidomimetic was synthetically generated and prepared
VHSEGTFTSDFF

SEQ ID NO: 194 - protein, 12 aa, Artificial
  This peptidomimetic was synthetically generated and prepared
VHAEGTFTSDFF

SEQ ID NO: 195 - protein, 12 aa, Artificial
  This peptidomimetic was synthetically generated and prepared
VHAEGTFTSDFF

SEQ ID NO: 196 - protein, 12 aa, Artificial
  This peptidomimetic was synthetically generated and prepared
VHPEGTFTSDFF

SEQ ID NO: 197 - protein, 12 aa, Artificial
  This peptidomimetic was synthetically generated and prepared
VHSQGTFTSDFF

SEQ ID NO: 198 - protein, 12 aa, Artificial
  This peptidomimetic was synthetically generated and prepared
VHAQGTFTSDFF

SEQ ID NO: 199 - protein, 12 aa, Artificial
  This peptidomimetic was synthetically generated and prepared
VHAQGTFTSDFF

SEQ ID NO: 200 - protein, 12 aa, Artificial
  This peptidomimetic was synthetically generated and prepared
VHPQGTFTSDFF

SEQ ID NO: 201 - protein, 12 aa, Artificial
  This peptidomimetic was synthetically generated and prepared
VHSEGTFTSDFF

SEQ ID NO: 202 - protein, 12 aa, Artificial
  This peptidomimetic was synthetically generated and prepared
VHAEGTFTSDFF

SEQ ID NO: 203 - protein, 12 aa, Artificial
  This peptidomimetic was synthetically generated and prepared
VHAEGTFTSDFF

SEQ ID NO: 204 - protein, 12 aa, Artificial
  This peptidomimetic was synthetically generated and prepared
VHPEGTFTSDFF

SEQ ID NO: 205 - protein, 13 aa, Artificial
  This peptidomimetic was synthetically generated and prepared
VVHSQGTFTSDFF

SEQ ID NO: 206 - protein, 13 aa, Artificial
  This peptidomimetic was synthetically generated and prepared
VVHAQGTFTSDFF

SEQ ID NO: 207 - protein, 13 aa, Artificial
  This peptidomimetic was synthetically generated and prepared
VVHAQGTFTSDFF

SEQ ID NO: 208 - protein, 13 aa, Artificial
  This peptidomimetic was synthetically generated and prepared
VVHPQGTFTSDFF

SEQ ID NO: 209 - protein, 13 aa, Artificial
  This peptidomimetic was synthetically generated and prepared
VVHSEGTFTSDFF

SEQ ID NO: 210 - protein, 13 aa, Artificial
  This peptidomimetic was synthetically generated and prepared
VVHAEGTFTSDFF

SEQ ID NO: 211 - protein, 13 aa, Artificial
  This peptidomimetic was synthetically generated and prepared
VVHAEGTFTSDFF

SEQ ID NO: 212 - protein, 13 aa, Artificial
  This peptidomimetic was synthetically generated and prepared
VVHPEGTFTSDFF

SEQ ID NO: 213 - protein, 13 aa, Artificial
  This peptidomimetic was synthetically generated and prepared
VVHSQGTFTSDFF

SEQ ID NO: 214 - protein, 13 aa, Artificial
  This peptidomimetic was synthetically generated and prepared
VVHAQGTFTSDFF

SEQ ID NO: 215 - protein, 13 aa, Artificial
  This peptidomimetic was synthetically generated and prepared
VVHAQGTFTSDFF

SEQ ID NO: 216 - protein, 13 aa, Artificial
  This peptidomimetic was synthetically generated and prepared
VVHPQGTFTSDFF

SEQ ID NO: 217 - protein, 13 aa, Artificial
  This peptidomimetic was synthetically generated and prepared
VVHSEGTFTSDFF

SEQ ID NO: 218 - protein, 13 aa, Artificial
  This peptidomimetic was synthetically generated and prepared
VVHAEGTFTSDFF

SEQ ID NO: 219 - protein, 13 aa, Artificial
  This peptidomimetic was synthetically generated and prepared
VVHAEGTFTSDFF

SEQ ID NO: 220 - protein, 13 aa, Artificial
  This peptidomimetic was synthetically generated and prepared
VVHPEGTFTSDFF

SEQ ID NO: 221 - protein, 13 aa, Artificial
  This peptidomimetic was synthetically generated and prepared
VVHSQGTFTSDFF

SEQ ID NO: 222 - protein, 13 aa, Artificial
  This peptidomimetic was synthetically generated and prepared
VVHAQGTFTSDFF

SEQ ID NO: 223 - protein, 13 aa, Artificial
  This peptidomimetic was synthetically generated and prepared
VVHAQGTFTSDFF

SEQ ID NO: 224 - protein, 13 aa, Artificial
  This peptidomimetic was synthetically generated and prepared
VVHPQGTFTSDFF

SEQ ID NO: 225 - protein, 13 aa, Artificial
  This peptidomimetic was synthetically generated and prepared
VVHSEGTFTSDFF

SEQ ID NO: 226 - protein, 13 aa, Artificial
  This peptidomimetic was synthetically generated and prepared
VVHAEGTFTSDFF

SEQ ID NO: 227 - protein, 13 aa, Artificial
  This peptidomimetic was synthetically generated and prepared
VVHAEGTFTSDFF

SEQ ID NO: 228 - protein, 13 aa, Artificial
  This peptidomimetic was synthetically generated and prepared
VVHPEGTFTSDFF